(19) United States
(12) Patent Application Publication
(10) Pub. No.: US 2006/0052382 A1
(43) Pub. Date: Mar. 9, 2006

(54) 3-Amino-4-phenylbutanoic acid derivatives as dipeptidyl peptidase inhibitors for the treatment or prevention of diabetes

(76) Inventors: Joseph L Duffy, Cranford, NJ (US); Scott D Edmondson, New York, NY (US); Dooseop Kim, Westfield, NJ (US); Brian A Kirk, Basking Ridge, NJ (US); Liping Wang, Dayton, NJ (US); Ann E Weber, Scotch Plains, NJ (US)

Correspondence Address: MERCK AND CO., INC, P O BOX 2000, RAHWAY, NJ, 07065-0907, US

(21) Appl. No.: 10/540,283

(22) PCT Filed: Dec. 16, 2003
(86) PCT No.: PCT/US03/40114

§ 371 Date: Jun. 20, 2005

Related U.S. Application Data
(60) Provisional application No. 60/435,389, filed on Dec. 20, 2002.
(60) Provisional application No. 60/469,315, filed on May 9, 2003.

(51) Int. Cl. A61K 31/498; C07D 487/02

(52) U.S. Cl. 514/249; 544/350

(57) ABSTRACT

The present invention is directed to 3-amino-4-phenylbutanoic acid derivatives which are inhibitors of the dipeptidyl peptidase-IV enzyme (“DP-IV inhibitors”) and which are useful in the treatment or prevention of diseases in which the dipeptidyl peptidase-IV enzyme is involved, such as diabetes and particularly type 2 diabetes. The invention is also directed to pharmaceutical compositions comprising these compounds and the use of these compounds and compositions in the prevention or treatment of such diseases in which the dipeptidyl peptidase-IV enzyme is involved.

### BACKGROUND OF THE INVENTION

Diabetes refers to a disease process derived from multiple causative factors and characterized by elevated levels of plasma glucose or hyperglycemia in the fasting state or after administration of glucose during an oral glucose tolerance test. Persistent or uncontrolled hyperglycemia is associated with increased and premature morbidity and mortality. Often abnormal glucose homeostasis is associated both directly and indirectly with alterations of the lipid, lipoprotein and apolipoprotein metabolism and other metabolic and hemodynamic disease. Therefore patients with Type 2 diabetes mellitus are at especially increased risk of macrovascular and microvascular complications, including coronary heart disease, stroke, peripheral vascular disease, hypertension, nephropathy, neuropathy, and retinopathy. Therefore, therapeutical control of glucose homeostasis, lipid metabolism and hypertension are critically important in the clinical management and treatment of diabetes mellitus.

There are two generally recognized forms of diabetes. In type 1 diabetes, or insulin-dependent diabetes mellitus (IDDM), patients produce little or no insulin, the hormone which regulates glucose utilization. In type 2 diabetes, or noninsulin dependent diabetes mellitus (NIDDM), patients often have plasma insulin levels that are the same or even elevated compared to nondiabetic subjects; however, these patients have developed a resistance to the insulin stimulating effect on glucose and lipid metabolism in the main insulin-sensitive tissues, which are muscle, liver and adipose tissues, and the plasma insulin levels, while elevated, are insufficient to overcome the pronounced insulin resistance.

Insulin resistance is not primarily due to a diminished number of insulin receptors but to a post-insulin receptor binding defect that is not yet understood. This resistance to insulin responsiveness results in insufficient insulin activation of glucose uptake, oxidation and storage in muscle and inadequate insulin repression of lipolysis in adipose tissue and of glucose production and secretion in the liver.

The available treatments for type 2 diabetes, which have not changed substantially in many years, have recognized limitations. While physical exercise and reductions in dietary intake of calories will dramatically improve the diabetic condition, compliance with this treatment is very poor because of well-entrenched sedentary lifestyles and excess food consumption, especially of foods containing high amounts of saturated fat. Increasing the plasma level of insulin by administration of sulfonylureas (e.g. tolbutamide and glipizide) or meglitinide, which stimulate the pancreatic β-cells to secrete more insulin, and/or by injection of insulin when sulfonylureas or meglitinide become ineffective, can result in insulin concentrations high enough to stimulate the very insulin-resistant tissues. However, dangerously low levels of plasma glucose can result from administration of insulin or insulin secretagogues (sulfonylureas or meglitinide), and an increased level of insulin resistance due to the even higher plasma insulin levels can occur. The biguanides increase insulin sensitivity resulting in some correction of hyperglycemia. However, the two biguanides, phenformin and metformin, can induce lactic acidosis and nausea/diarrhea. Metformin has fewer side effects than phenformin and is often prescribed for the treatment of Type 2 diabetes.

The glitazones (i.e. 5-benzylthiazolidine-2,4-diones) are a more recently described class of compounds with potential for ameliorating many symptoms of type 2 diabetes. These agents substantially increase insulin sensitivity in muscle, liver and adipose tissue in several animal models of type 2 diabetes resulting in partial or complete correction of the elevated plasma levels of glucose without occurrence of hypoglycemia. The glitazones that are currently marketed are agonists of the peroxisome proliferator activated receptor (PPAR), primarily the PPAR-gamma subtype. PPAR-gamma agonism is generally believed to be responsible for the improved insulin sensititization that is observed with the glitazones. Newer PPAR agonists that are being tested for treatment of Type II diabetes are agonists of the alpha, gamma or delta subtype, or a combination of these, and in many cases are chemically different from the glitazones (i.e., they are not thiazolidinediones). Serious side effects (e.g. liver toxicity) have occurred with some of the glitazones, such as troglitazone.

Additional methods of treating the disease are still under investigation. New biochemical approaches that have been recently introduced or are still under development include treatment with alpha-glucosidase inhibitors (e.g. acarbose) and protein tyrosine phosphatase-1B (PTP-1B) inhibitors.

Compounds that are inhibitors of the dipeptidyl peptidase-IV (“DP-IV” or “DPP-IV”) enzyme are also under investigation as drugs that may be useful in the treatment of diabetes, and particularly type 2 diabetes. See for example WO 97/40832, WO 98/19998, U.S. Pat. No. 5,939,560, Bioorg. Med. Chem. Lett., 6: 1163-1166 (1996); and Bioorg. Med. Chem. Lett., 6: 2745-2748 (1996). The usefulness of DP-IV inhibitors in the treatment of type 2 diabetes is based on the fact that DP-IV in vivo readily inactivates glucagon like peptide-1 (GLP-1) and gastric inhibitory peptide (GIP). GLP-1 and GIP are incretins and are produced when food is consumed. The incretins stimulate production of insulin. Inhibition of DP-IV leads to decreased inactivation of the incretins, and this in turn results in increased effectiveness of the incretins in stimulating production of insulin by the pancreas. DP-IV inhibition therefore results in an increased level of serum insulin. Advantageously, since the incretins are produced by the body only when food is consumed, DP-IV inhibition is not expected to increase the level of insulin at inappropriate times, such as between meals, which can lead to excessively low blood sugar (hypoglycemia). Inhibition of DP-IV is therefore expected to increase insulin without increasing the risk of hypoglycemia, which is a dangerous side effect associated with the use of insulin secretagogues.

DP-IV inhibitors also have other therapeutic utilities, as discussed herein. DP-IV inhibitors have not been studied extensively to date, especially for utilities other than diabetes. New compounds are needed so that improved DP-IV inhibitors can be found for the treatment of diabetes and potentially other diseases and conditions.

### SUMMARY OF THE INVENTION

The present invention is directed to 3-amino-4-phenylbutanoic acid derivatives which are inhibitors of the dipeptidyl peptidase-IV enzyme (“DP-IV inhibitors”) and which are useful in the treatment or prevention of diseases in which the dipeptidyl peptidase-IV enzyme is involved, such as diabetes and particularly type 2 diabetes. The invention is also directed to pharmaceutical compositions comprising these compounds and the use of these compounds and compositions in the prevention or treatment of such diseases in which the dipeptidyl peptidase-IV enzyme is involved.

### DETAILED DESCRIPTION OF THE INVENTION

The present invention relates to 3-amino-4-phenylbutanoic acid derivatives useful as inhibitors of dipeptidyl peptidase-IV. Compounds of the present invention are described by structural formula I: 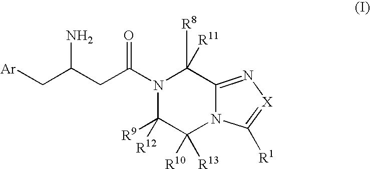
or a pharmaceutically acceptable salt thereof; wherein

each n is independently 0, 1, or 2;

X is N or CR²;

Ar is phenyl substituted with one to five R³ substituents;

R¹ and R² are each independently selected from the group consisting of
hydrogen,
halogen,
hydroxy,
cyano,
C₁₋₁₀ alkyl, wherein alkyl is unsubstituted or substituted with one to five substituents independently selected from halogen or hydroxy,
C₁₋₁₀ alkoxy, wherein alkoxy is unsubstituted or substituted with one to five substituents independently selected from halogen or hydroxy,
C₁₋₁₀ alkylthio, wherein alkylthio is unsubstituted or substituted with one to five substituents independently selected from halogen or hydroxy,
C₂₋₁₀ alkenyl, wherein alkenyl is unsubstituted or substituted with one to five substituents independently selected from halogen or hydroxy,
(CH₂)ₙCOOH,
(CH₂)ₙCOOC₁₋₆ alkyl,
(CH₂)ₙCONR⁴R⁵, wherein R⁴ and R⁵ are independently selected from the group consisting of hydrogen, tetrazolyl, thiazolyl, (CH₂)ₙ-phenyl, (CH₂)ₙ—C₃₋₆ cycloalkyl, and C₁₋₆ alkyl, wherein alkyl is unsubstituted or substituted with one to five halogens and wherein phenyl and cycloalkyl are unsubstituted or substituted with one to five substituents independently selected from halogen, hydroxy, C₁₋₆ alkyl, and C₁₋₆ alkoxy, wherein alkyl and alkoxy are unsubstituted or substituted with one to five halogens;
   or R⁴ and R⁵ together with the nitrogen atom to which they are attached form a heterocyclic ring selected from azetidine, pyrrolidine, piperidine, piperazine, and morpholine wherein said heterocyclic ring is unsubstituted or substituted with one to five substituents independently selected from halogen, hydroxy, C₁₋₆ alkyl, and C₁₋₆ alkoxy, wherein alkyl and alkoxy are unsubstituted or substituted with one to five halogens;
(CH₂)ₙ—NR⁴R⁵,
(CH₂)ₙ—OCONR⁴R⁵,
(CH₂)ₙ—SO₂NR⁴R⁵,
(CH₂)ₙ—SO₂R⁶,
(CH₂)ₙ—NR⁷SO₂R⁶,
(CH₂)ₙ—NR⁷CONR⁴R⁵,
(CH₂)ₙ—NR⁷COR⁷,
(CH₂)ₙ—NR⁷CO₂R⁶,
(CH₂)ₙ—COR⁶,
(CH₂)ₙ—C₃₋₆ cycloalkyl, wherein cycloalkyl is unsubstituted or substituted with one to three substituents independently selected from halogen, hydroxy, C₁₋₆ alkyl, and C₁₋₆ alkoxy, wherein alkyl and alkoxy are unsubstituted or substituted with one to five halogens,
(CH₂)ₙ-aryl, wherein aryl is unsubstituted or substituted with one to five substituents independently selected from halogen, cyano, hydroxy, NR⁷SO₂R⁶, SO₂R⁶, CO₂H, C₁₋₆ alkyloxycarbonyl, C₁₋₆ alkyl, and C₁₋₆ alkoxy, wherein alkyl and alkoxy are unsubstituted or substituted with one to five halogens,
(CH₂)ₙ-heteroaryl, wherein heteroaryl is unsubstituted or substituted with one to three substituents independently selected from hydroxy, halogen, C₁₋₆ alkyl, and C₁₋₆ alkoxy, wherein alkyl and alkoxy are unsubstituted or substituted with one to five halogens, and
(CH₂)ₙ-heterocyclyl, wherein heterocyclyl is unsubstituted or substituted with one to three substituents independently selected from oxo, hydroxy, halogen, C₁₋₆ alkyl, and C₁₋₆ alkoxy, wherein alkyl and alkoxy are unsubstituted or substituted with one to five halogens,
wherein any methylene (CH₂) carbon atom in R¹ or R² is unsubstituted or substituted with one to two groups independently selected from halogen, hydroxy, and C₁₋₄ alkyl unsubstituted or substituted with one to five halogens;

each R³ is independently selected from the group consisting of
hydrogen,
halogen,
cyano,
hydroxy,
C₁₋₆ alkyl, unsubstituted or substituted with one to five halogens, and
C₁₋₆ alkoxy, unsubstituted or substituted with one to five halogens;

R⁶ is independently selected from the group consisting of tetrazolyl, thiazolyl, (CH₂)ₙ-phenyl, (CH₂)ₙ—C₃₋₆ cycloalkyl, and C₁₋₆ alkyl, wherein alkyl is unsubstituted or substituted with one to five halogens and wherein phenyl and cycloalkyl are unsubstituted or substituted with one to five substituents independently selected from halogen, hydroxy, C₁₋₆ alkyl, and C₁₋₆ alkoxy, wherein alkyl and alkoxy are unsubstituted or substituted with one to five halogens, and wherein any methylene (CH₂) carbon atom in R⁶ is unsubstituted or substituted with one to two groups independently selected from halogen, hydroxy, C₁₋₄ alkyl, and C₁₋₄ alkoxy, wherein alkyl and alkoxy are unsubstituted or substituted with one to five halogens;

each R⁷ is hydrogen or R⁶;

R⁸, R⁹ and R¹⁰ are each independently selected from the group consisting of
hydrogen,
cyano,
carboxy,
C₁₋₆ alkyloxycarbonyl,
C₁₋₁₀ alkyl, unsubstituted or substituted with one to five substituents independently selected from halogen, hydroxy, C₁₋₆ alkoxy; carboxy, C₁₋₆ alkyloxycarbonyl, and phenyl-C₁₋₃ alkoxy, wherein alkoxy is unsubstituted or substituted with one to five halogens,
(CH₂)ₙ-aryl, wherein aryl is unsubstituted or substituted with one to five substituents independently selected from halogen, hydroxy, C₁₋₆ alkyl, and C₁₋₆ alkoxy, wherein alkyl and alkoxy are unsubstituted or substituted with one to five halogens,
(CH₂)ₙ-heteroaryl, wherein heteroaryl is unsubstituted or substituted with one to three substituents independently selected from hydroxy, halogen, C₁₋₆ alkyl, and C₁₋₆ alkoxy, wherein alkyl and alkoxy are unsubstituted or substituted with one to five halogens,
(CH₂)ₙ-heterocyclyl, wherein heterocyclyl is unsubstituted or substituted with one to three substituents independently selected from oxo, hydroxy, halogen, C₁₋₆ alkyl, and C₁₋₆ alkoxy, wherein alkyl and alkoxy are unsubstituted or substituted with one to five halogens,
(CH₂)ₙ—C₃₋₆ cycloalkyl, wherein cycloalkyl is unsubstituted or substituted with one to three substituents independently selected from halogen, hydroxy, C₁₋₆ alkyl, and C₁₋₆ alkoxy, wherein alkyl and alkoxy are unsubstituted or substituted with one to five halogens, and
(CH₂)ₙCONR⁴R⁵, wherein R⁴ and R⁵ are independently selected from the group consisting of hydrogen, tetrazolyl, thiazolyl, (CH₂)ₙ-phenyl, (CH₂)ₙ—C₃₋₆ cycloalkyl, and C₁₋₆ alkyl, wherein alkyl is unsubstituted or substituted with one to five halogens and wherein phenyl and cycloalkyl are unsubstituted or substituted with one to five substituents independently selected from halogen, hydroxy, C₁₋₆ alkyl, and C₁₋₆ alkoxy, wherein alkyl and alkoxy are unsubstituted or substituted with one to five halogens;
   or R⁴ and R⁵ together with the nitrogen atom to which they are attached form a heterocyclic ring selected from azetidine, pyrrolidine, piperidine, piperazine, and morpholine wherein said heterocyclic ring is unsubstituted or substituted with one to five substituents independently selected from halogen, hydroxy, C₁₋₆ alkyl, and C₁₋₆ alkoxy, wherein alkyl and alkoxy are unsubstituted or substituted with one to five halogens;
wherein any methylene (CH₂) carbon atom in R⁸, R⁹ or R¹⁰ is unsubstituted or substituted with one to two groups independently selected from halogen, hydroxy, and C₁₋₄ alkyl unsubstituted or substituted with one to five halogens;

with the proviso that when X is N, R¹⁰, R¹¹, R¹² and R¹³ are hydrogen,

R⁸ or R⁹ is
hydrogen;
cyano;
C₁₋₁₀ alkyl, unsubstituted or substituted with one to five substituents selected from:
   (1) halogen,
   (2) hydroxy,
   (3) phenyl, optionally substituted with one to five substituents independently selected from halogen, hydroxy, C₁₋₆ alkyl, and C₁₋₆ alkoxy, wherein alkyl and alkoxy are optionally substituted with one to five halogens,
   (4) naphthyl, optionally substituted with one to five substituents independently selected from halogen, hydroxy, C₁₋₆ alkyl, and C₁₋₆ alkoxy, wherein alkyl and alkoxy are optionally substituted with one to five halogens,
   (5) CO₂H,
   (6) CO₂C₁₋₆ alkyl,
   (7) CONR¹¹R¹², wherein R¹¹ and R¹² are independently selected from the group consisting of hydrogen, tetrazolyl, phenyl, C₃₋₆ cycloalkyl and C₁₋₆ alkyl, wherein alkyl is optionally substituted with one to six substituents independently selected from halogen and phenyl, wherein the phenyl or C₃₋₆ cycloalkyl being R¹¹ or R¹² or the optional phenyl substituent on C₁₋₆ alkyl are optionally substituted with one to five substituents independently selected from halogen, hydroxy, C₁₋₆ alkyl, and C₁₋₆ alkoxy, said C₁₋₆ alkyl and C₁₋₆ alkoxy being optionally substituted with one to five halogens,
   or wherein R¹¹ and R¹² are optionally joined to form a ring selected from pyrrolidine, piperidine and morpholine;
phenyl, which is unsubstituted or substituted with one to five substituents independently selected from C₁₋₆ alkyl, C₁₋₆ alkoxy, hydroxy, and halogen, wherein alkyl and alkoxy are optionally substituted with one to five halogens;
naphthyl, which is unsubstituted or substituted with one to five substituents independently selected from C₁₋₆ alkyl, C₁₋₆ alkoxy, hydroxy, and halogen, wherein alkyl and alkoxy are optionally substituted with one to five halogens;
CO₂H;
C₁₋₆ alkyloxycarbonyl;
CONR¹¹R¹²; or
C₃₋₆ cycloalkyl, which is optionally substituted with one to five substituents independently selected from halogen, hydroxy, C₁₋₆ alkyl, and C₁₋₆ alkoxy, wherein alkyl and alkoxy are optionally substituted with one to five halogens; and when X is CR² and

R² is
hydrogen,
cyano,
C₁₋₁₀ alkyl, unsubstituted or substituted with one to five halogens,
(CH₂)ₙ-phenyl, which is unsubstituted or substituted with one to five substituents independently selected from halogen, cyano hydroxy, R¹³, OR¹³, NHSO₂R¹³, SO₂R¹³, CO₂H, and C₁₋₆ alkyloxycarbonyl, wherein R¹³ is C₁₋₆ alkyl, unsubstituted or substituted with one to five halogens; or
a 5- or 6-membered heterocycle which may be saturated or unsaturated comprising one to four heteroatoms independently selected from N, S and O, the heterocycle being unsubstituted or substituted with one to three substituents independently selected from oxo, hydroxy, halogen,
   C₁₋₆ alkyl, and C₁₋₆ alkoxy, wherein alkyl and alkoxy are optionally substituted with one to five halogens;

then in both cases R¹ is not
(1) hydrogen,
(2) cyano,
(3) C₁₋₁₀ alkyl, unsubstituted or substituted with one to five halogens,
(4) (CH₂)ₙ-phenyl, which is unsubstituted or substituted with one to five substituents independently selected from halogen, cyano hydroxy, R¹³, OR_{13,} NHSO₂R¹³, SO₂R¹³, CO₂H, and C₁₋₆ alkyloxycarbonyl, wherein R¹³ is C₁₋₆ alkyl, unsubstituted or substituted with one to five halogens; or
(5) a 5- or 6-membered heterocycle which may be saturated or unsaturated comprising one to four heteroatoms independently selected from N, S and O, the heterocycle being unsubstituted or substituted with one to three substituents independently selected from oxo, hydroxy, halogen, C₁₋₆ alkyl, and C₁₋₆ alkoxy, wherein alkyl and alkoxy are optionally substituted with one to five halogens.

R₁, R¹² and R¹³ are each independently hydrogen or C₁₋₆ alkyl.

In one embodiment of the compounds of the present invention, the carbon atom marked with an * has the R configuration as depicted in formula Ia 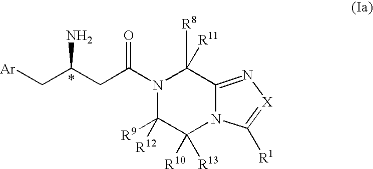
wherein Ar, X, R¹, R⁸, R⁹, R¹⁰, R¹¹, R¹², and R¹³ are as defined herein.

In a second embodiment of the compounds of the present invention, X is N as depicted in formula Ib: 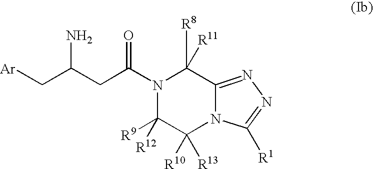
wherein Ar, R¹, R⁸, R⁹, R¹⁰, R¹¹, R¹², and R¹³ are as defined herein.

In a class of this second embodiment, the carbon atom marked with an * has the R configuration as depicted in formula Ic: 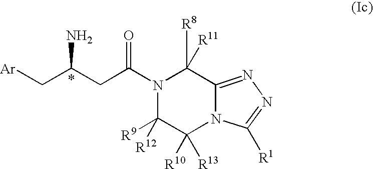
wherein Ar, R¹, R⁸, R⁹, R¹⁰, R¹¹, R¹², and R¹³ are as defined herein.

In another class of this second embodiment of the compounds of the present invention, R⁹, R¹⁰, R¹¹, R¹², and R¹³ are hydrogen as depicted in formula Id: 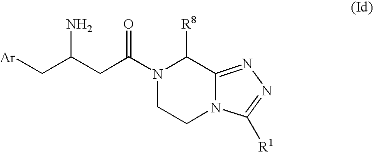
wherein Ar, R¹, and R⁸ are as defined herein.

In a subclass of this class, R⁸ is hydrogen.

In a third embodiment of the compounds of the present invention, X is CR² as depicted in formula Ie: 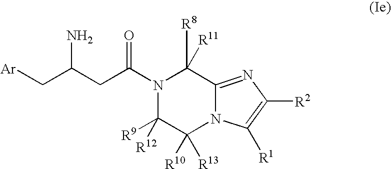
wherein Ar, R¹, R², R⁸, R⁹, R¹⁰, R₁₁, R¹², and R¹³ are as defined herein.

In a class of this third embodiment, the carbon atom marked with an * has the R configuration as depicted in formula If: 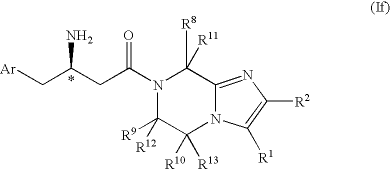
wherein Ar, R¹, R², R⁸, R⁹, R¹⁰, R¹¹, R¹², and R¹³ are as defined herein.

In another class of this third embodiment of the compounds of the present invention, R⁹, R¹⁰, R¹¹, R¹², and R¹³ are hydrogen as depicted in formula Ig: 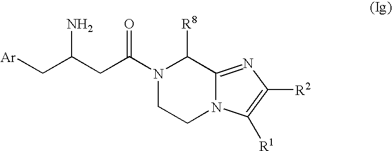
wherein Ar, R¹, R², and R⁸ are as defined herein.

In a subclass of this class, R⁸ is hydrogen.

In a fourth embodiment of the compounds of the present invention, R³ is selected from the group consisting of hydrogen, fluoro, chloro, bromo, trifluoromethyl, and methyl. In a class of this embodiment, R³ is selected from the group consisting of hydrogen, fluoro, and chloro. In a subclass of this class, R³ is hydrogen or fluoro.

In a fifth embodiment of the compounds of the present invention, R¹ is selected from the group consisting of:
hydrogen,
halogen,
C₁₋₆ alkyl, wherein alkyl is unsubstituted or substituted with one to five substituents independently selected from halogen or hydroxy,
C₁₋₆ alkoxy, wherein alkoxy is unsubstituted or substituted with one to five substituents independently selected from halogen or hydroxy,
C₁₋₆ alkylthio, wherein alkylthio is unsubstituted or substituted with one to five substituents independently selected from halogen or hydroxy,
C₂₋₆ alkenyl, wherein alkenyl is unsubstituted or substituted with one to five substituents independently selected from halogen or hydroxy,
(CH₂)ₙCOOH,
(CH₂)ₙCOOC₁₋₆ alkyl,
(CH₂)ₙCONR⁴R⁵, wherein R⁴ and R⁵ are independently selected from the group consisting of hydrogen, tetrazolyl, thiazolyl, (CH₂)ₙ-phenyl, (CH₂)ₙ—C₃₋₆ cycloalkyl, and C₁₋₆ alkyl, wherein alkyl is unsubstituted or substituted with one to five halogens and wherein phenyl and cycloalkyl are unsubstituted or substituted with one to five substituents independently selected from halogen, hydroxy, C₁₋₆ alkyl, and C₁₋₆ alkoxy, wherein alkyl and alkoxy are unsubstituted or substituted with one to five halogens;
   or R⁴ and R⁵ together with the nitrogen atom to which they are attached form a heterocyclic ring selected from azetidine, pyrrolidine, piperidine, piperazine, and morpholine wherein said heterocyclic ring is unsubstituted or substituted with one to five substituents independently selected from halogen, hydroxy, C₁₋₆ alkyl, and C₁₋₆ alkoxy, wherein alkyl and alkoxy are unsubstituted or substituted with one to five halogens,
(CH₂)ₙ—NR⁴R⁵,
(CH₂)ₙ—NR⁷COR⁷,
(CH₂)ₙ—C₃₋₆ cycloalkyl, wherein cycloalkyl is unsubstituted or substituted with one to three substituents independently selected from halogen, hydroxy, C₁₋₆ alkyl, and C₁₋₆ alkoxy, wherein alkyl and alkoxy are unsubstituted or substituted with one to five halogens, and
(CH₂)ₙ-aryl, wherein aryl is unsubstituted or substituted with one to five substituents independently selected from halogen, CN, hydroxy, NR⁷SO₂R⁶, SO₂R⁶, CO₂H, C₁₋₆ alkyloxycarbonyl, C₁₋₆ alkyl, and C₁₋₆ alkoxy, wherein alkyl and alkoxy are unsubstituted or substituted with one to five halogens;
wherein any methylene (CH₂) carbon atom in R¹ or R² is unsubstituted or substituted with one to two groups independently selected from halogen, hydroxy, and C₁₋₄ alkyl unsubstituted or substituted with one to five halogens.

In a class of this embodiment of the compounds of the present invention, R¹ is selected from the group consisting of
hydrogen,
methyl,
ethyl,
trifluoromethyl,
CH₂CF₃,
CF₂CF₃,
phenyl,
cyclopropyl,
fluoro,
chloro,
bromo,
vinyl,
amino,
isopropylamino,
acetylamino,
2,2,2-trifluoroacetylamino,
tert-butylaminocarbonyl,
ethoxycarbonyl,
carboxy,
1-hydroxyethyl,
methoxy,
isopropoxy, and
methylthio.

In a sixth embodiment of the compounds of the present invention, R² is selected from the group consisting of
hydrogen,
halogen,
C₁₋₆ alkyl, wherein alkyl is unsubstituted or substituted with one to five substituents independently selected from halogen or hydroxy,
C₂₋₆ alkenyl, wherein alkenyl is unsubstituted or substituted with one to five substituents independently selected from halogen or hydroxy,
(CH₂)ₙCOOH,
(CH₂)ₙCOOC₁₋₆ alkyl,
(CH₂)ₙCONR⁴R⁵, wherein R⁴ and R⁵ are independently selected from the group consisting of hydrogen, tetrazolyl, thiazolyl, (CH₂)ₙ-phenyl, (CH₂)ₙ—C₃₋₆ cycloalkyl, and C₁₋₆ alkyl, wherein alkyl is unsubstituted or substituted with one to five halogens and wherein phenyl and cycloalkyl are unsubstituted or substituted with one to five substituents independently selected from halogen, hydroxy, C₁₋₆ alkyl, and C₁₋₆ alkoxy, wherein alkyl and alkoxy are unsubstituted or substituted with one to five halogens;
   or R⁴ and R⁵ together with the nitrogen atom to which they are attached form a heterocyclic ring selected from azetidine, pyrrolidine, piperidine, piperazine, and morpholine wherein said heterocyclic ring is unsubstituted or substituted with one to five substituents independently selected from halogen, hydroxy, C₁₋₆ alkyl, and C₁₋₆ alkoxy, wherein alkyl and alkoxy are unsubstituted or substituted with one to five halogens,
(CH₂)ₙ—NR⁴R⁵,
(CH₂)ₙ—NR⁷COR⁷,
(CH₂)ₙ—COR⁶,
(CH₂)ₙ—C₃₋₆ cycloalkyl, wherein cycloalkyl is unsubstituted or substituted with one to three substituents independently selected from halogen, hydroxy, C₁₋₆ alkyl, and C₁₋₆ alkoxy, wherein alkyl and alkoxy are unsubstituted or substituted with one to five halogens, and
(CH₂)ₙ-aryl, wherein aryl is unsubstituted or substituted with one to five substituents independently selected from halogen, cyano, hydroxy, NR₇SO₂R⁶, SO₂R⁶, CO₂H, C₁₋₆ alkyloxycarbonyl, C₁₋₆ alkyl, and C₁₋₆ alkoxy, wherein alkyl and alkoxy are unsubstituted or substituted with one to five halogens;
wherein any methylene (CH₂) carbon atom in R¹ or R² is unsubstituted or substituted with one to two groups independently selected from halogen, hydroxy, and C₁₋₄ alkyl unsubstituted or substituted with one to five halogens.

In a class of this embodiment of the compounds of the present invention, R² is selected from the group consisting of:
hydrogen
trifluoromethyl,
phenyl,
cyclopropyl,
carboxy,
ethoxycarbonyl,
dimethylaminocarbonyl,
aminocarbonyl,
morpholin-4-ylcarbonyl,
tert-butylaminocarbonyl,
cyclopropylcarbonyl,
tetrazol-5-ylaminocarbonyl, and
2,2,2-trifluoroacetylamino.

In a seventh embodiment of the compounds of the present invention, R⁸, R⁹, and R¹⁰ are independently selected from the group consisting of:
hydrogen,
C₁₋₆ alkyl, unsubstituted or substituted with one to five substituents independently selected from halogen, hydroxy, C₁₋₆ alkoxy, and phenyl-C₁₋₃ alkoxy, wherein alkoxy is unsubstituted or substituted with one to five halogens,
(CH₂)ₙ-phenyl, wherein phenyl is unsubstituted or substituted with one to five substituents independently selected from halogen, hydroxy, C₁₋₆ alkyl, and C₁₋₆ alkoxy, wherein alkyl and alkoxy are unsubstituted or substituted with one to five halogens,
(CH₂)ₙ-heteroaryl, wherein heteroaryl is unsubstituted or substituted with one to three substituents independently selected from hydroxy, halogen, C₁₋₆ alkyl, and C₁₋₆ alkoxy, wherein alkyl and alkoxy are unsubstituted or substituted with one to five halogens,
(CH₂)ₙ-heterocyclyl, wherein heterocyclyl is unsubstituted or substituted with one to three substituents independently selected from oxo, hydroxy, halogen, C₁₋₆ alkyl, and C₁₋₆ alkoxy, wherein alkyl and alkoxy are unsubstituted or substituted with one to five halogens, and
(CH₂)ₙ—C₃₋₆ cycloalkyl, wherein cycloalkyl is unsubstituted or substituted with one to three substituents independently selected from halogen, hydroxy, C₁₋₆ alkyl, and C₁₋₆ alkoxy, wherein alkyl and alkoxy are optionally substituted with one to five halogens;
wherein any methylene (CH₂) carbon atom in R⁸, R⁹, or R¹⁰ is unsubstituted or substituted with one to two groups independently selected from halogen, hydroxy, and C₁₋₄ alkyl unsubstituted or substituted with one to five halogens;
   and R₁₁, R¹², and R¹³ are each independently hydrogen or methyl.

In a class of this embodiment of the compounds of the present invention, R⁸, R⁹, and R¹⁰ are each independently selected from the group consisting of
hydrogen,
C₁₋₃ alkyl, unsubstituted or substituted with one to three substituents independently selected from halogen, hydroxy, C₁₋₆ alkoxy, and phenyl-C₁₋₃ alkoxy, wherein alkoxy is unsubstituted or substituted with one to five halogens,
(CH₂)ₙ-phenyl, wherein phenyl is unsubstituted or substituted with one to five substituents independently selected from halogen, hydroxy, C₁₋₆ alkyl, and C₁₋₆ alkoxy, wherein alkyl and alkoxy are unsubstituted or substituted with one to five halogens,
(CH₂)ₙ-heteroaryl, wherein heteroaryl is unsubstituted or substituted with one to three substituents independently selected from hydroxy, halogen, C₁₋₆ alkyl, and C₁₋₆ alkoxy, wherein alkyl and alkoxy are optionally substituted with one to five halogens, and
(CH₂)ₙ-heterocyclyl, wherein heterocyclyl is unsubstituted or substituted with one to three substituents independently selected from oxo, hydroxy, halogen, C₁₋₆ alkyl, and C₁₋₆ alkoxy, wherein alkyl and alkoxy are optionally substituted with one to five halogens,
(CH₂)ₙ—C₃₋₆ cyclopropyl;
wherein any methylene (CH₂) carbon atom in R⁸, R⁹, or R¹⁰ is unsubstituted or substituted with one to two groups independently selected from halogen, hydroxy, and C₁₋₄ alkyl unsubstituted or substituted with one to five halogens;
   and R¹¹, R¹², and R¹³ are each independently hydrogen or methyl.

In a subclass of this class, R⁸, R⁹, and R¹⁰ are each independently selected from the group consisting of

hydrogen,

CH₃,

CH₂CH₃,

CH₂-cyclopropyl,

CHF-cyclopropyl,

CH(OH)-cyclopropyl,

CH₂OCH₂Ph,

CH₂(4-F-Ph),

CH₂(4-CF₃-Ph), and

CH₂-[1,2,4]triazol-4-yl;

and R¹¹, R¹², and R¹³ are each independently hydrogen or methyl.

In a further subclass of this class, R⁹, R¹⁰, R¹², and R¹³ are hydrogen. In a subclass of this subclass, R⁸ and R¹¹ are hydrogen.

Illustrative, but nonlimiting examples, of compounds of the present invention that are useful as dipeptidyl peptidase-IV inhibitors are the following: 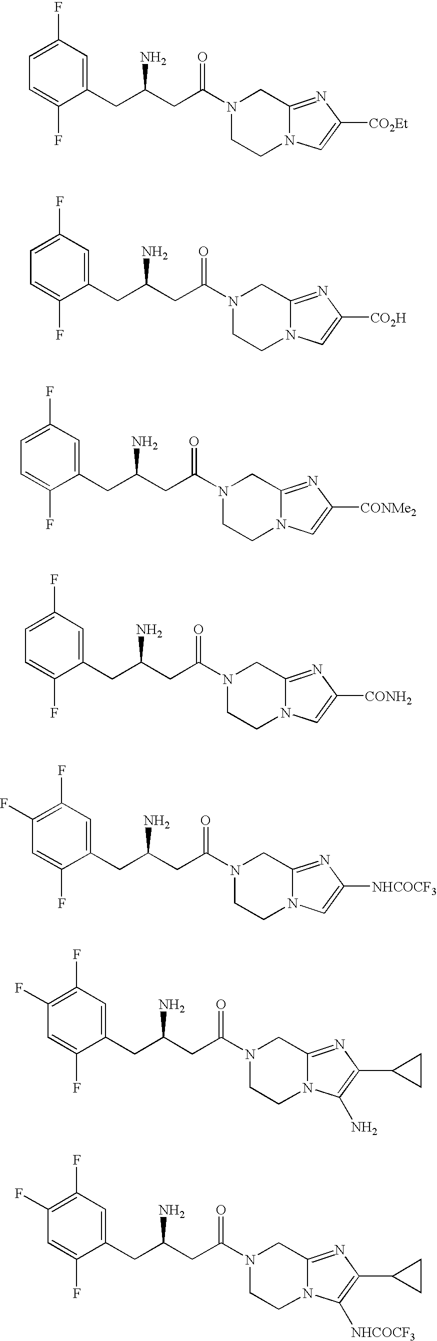 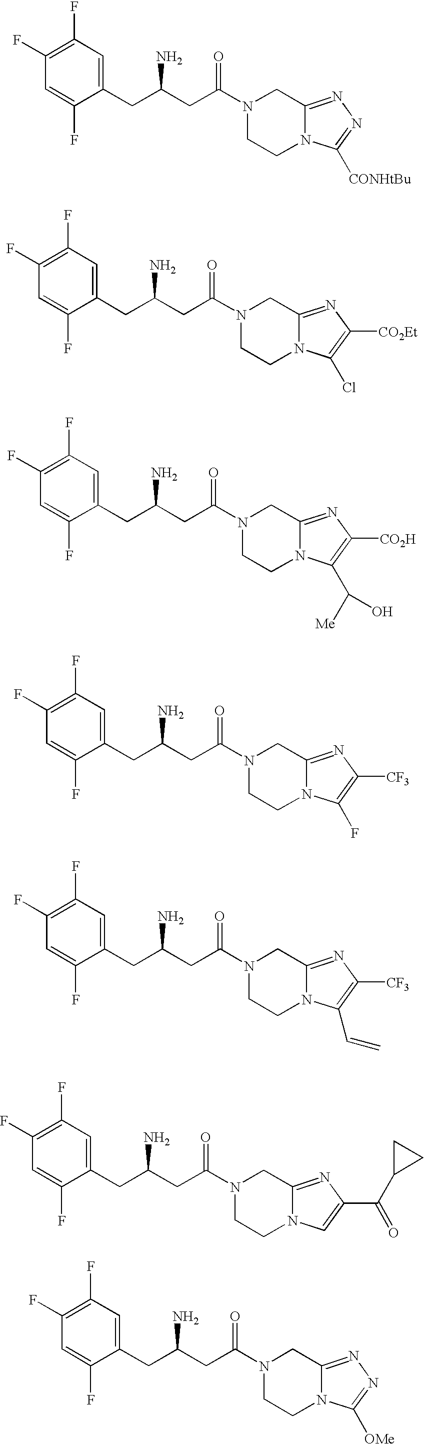 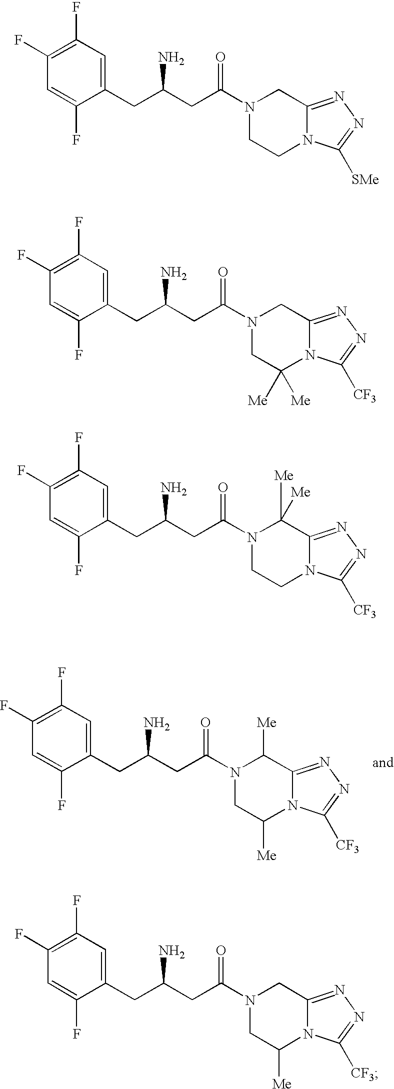
or a pharmaceutically acceptable salt thereof.

As used herein the following definitions are applicable.

“Alkyl”, as well as other groups having the prefix “alk”, such as alkoxy and alkanoyl, means carbon chains which may be linear or branched, and combinations thereof, unless the carbon chain is defined otherwise. Examples of alkyl groups include methyl, ethyl, propyl, isopropyl, butyl, sec- and tert-butyl, pentyl, hexyl, heptyl, octyl, nonyl, and the like. Where the specified number of carbon atoms permits, e.g., from C₃₋₁₀, the term alkyl also includes cycloalkyl groups, and combinations of linear or branched alkyl chains combined with cycloalkyl structures. When no number of carbon atoms is specified, C₁₋₆ is intended.

“Cycloalkyl” is a subset of alkyl and means a saturated carbocyclic ring having a specified number of carbon atoms. Examples of cycloalkyl include cyclopropyl, cyclobutyl, cyclopentyl, cyclohexyl, cycloheptyl, cyclooctyl, and the like. A cycloalkyl group generally is monocyclic unless stated otherwise. Cycloalkyl groups are saturated unless otherwise defined.

The term “alkoxy” refers to straight or branched chain alkoxides of the number of carbon atoms specified (e.g., C₁₋₁₀ alkoxy), or any number within this range [i.e., methoxy (MeO—), ethoxy, isopropoxy, etc.).

The term “alkylthio” refers to straight or branched chain alkylsulfides of the number of carbon atoms specified (e.g., C₁₋₁₀ alkylthio), or any number within this range [i.e., methylthio (MeS—), ethylthio, isopropylthio, etc.].

The term “alkylamino” refers to straight or branched alkylamines of the number of carbon atoms specified (e.g., C₁₋₆ alkylamino), or any number within this range [i.e., methylamino, ethylamino, isopropylamino, t-butylamino, etc.].

The term “alkylsulfonyl” refers to straight or branched chain alkylsulfones of the number of carbon atoms specified (e.g., C₁₋₆ alkylsulfonyl), or any number within this range [i.e., methylsulfonyl (MeSO₂—), ethylsulfonyl, isopropylsulfonyl, etc.].

The term “alkyloxycarbonyl” refers to straight or branched chain esters of a carboxylic acid derivative of the present invention of the number of carbon atoms specified (e.g., C₁₋₆ alkyloxycarbonyl), or any number within this range [i.e., methyloxycarbonyl (MeOCO—), ethyloxycarbonyl, or butyloxycarbonyl].

“Aryl” means a mono- or polycyclic aromatic ring system containing carbon ring atoms. The preferred aryls are monocyclic or bicyclic 6-10 membered aromatic ring systems. Phenyl and naphthyl are preferred aryls. The most preferred aryl is phenyl.

“Heterocycle” and “heterocyclyl” refer to saturated or unsaturated non-aromatic rings or ring systems containing at least one heteroatom selected from O, S and N, further including the oxidized forms of sulfur, namely SO and SO₂. Examples of heterocycles include tetrahydrofuran (THF), dihydrofuran, 1,4-dioxane, morpholine, 1,4-dithiane, piperazine, piperidine, 1,3-dioxolane, imidazolidine, imidazoline, pyrroline, pyrrolidine, tetrahydropyran, dihydropyran, oxathiolane, dithiolane, 1,3-dioxane, 1,3-dithiane, oxathiane, thiomorpholine, and the like.

“Heteroaryl” means an aromatic or partially aromatic heterocycle that contains at least one ring heteroatom selected from O, S and N. Heteroaryls also include heteroaryls fused to other kinds of rings, such as aryls, cycloalkyls and heterocycles that are not aromatic. Examples of heteroaryl groups include pyrrolyl, isoxazolyl, isothiazolyl, pyrazolyl, pyridinyl, 2-oxo-(1H)-pyridinyl (2-hydroxy-pyridinyl), oxazolyl, 1,2,4-oxadiazolyl, 1,3,4-oxadiazolyl, thiadiazolyl, thiazolyl, imidazolyl, triazolyl, tetrazolyl, furyl, triazinyl, thienyl, pyrimidinyl, pyrazinyl, benzisoxazolyl, benzoxazolyl, benzothiazolyl, benzothiadiazolyl, dihydrobenzofuranyl, indolinyl, pyridazinyl, indazolyl, isoindolyl, dihydrobenzothienyl, indolizinyl, cinnolinyl, phthalazinyl, quinazolinyl, naphthyridinyl, carbazolyl, benzodioxolyl, quinoxalinyl, purinyl, furazanyl, isobenzylfuranyl, benzimidazolyl, benzofuranyl, benzothienyl, quinolyl, indolyl, isoquinolyl, dibenzofuranyl, imidazo[1,2-a]pyridinyl, [1,2,4-triazolo][4,3-a]pyridinyl, pyrazolo[1,5-a]pyridinyl, [1,2,4-triazolo][1,5-a]pyridinyl, 2-oxo-1,3-benzoxazolyl 4-oxo-3H-quinazolinyl, 3-oxo-[1,2,4]-triazolo[4,3-a]-2H-pyridinyl, 5-oxo-[1,2,4]-4H-oxadiazolyl, 2-oxo-[1,3,4]-3H-oxadiazolyl, 2-oxo-1,3-dihydro-2H-imidazolyl, 3-oxo-2,4-dihydro-3H-1,2,4-triazolyl, and the like. For heterocyclyl and heteroaryl groups, rings and ring systems containing from 3-15 atoms are included, forming 1-3 rings.

“Halogen” refers to fluorine, chlorine, bromine and iodine. Chlorine and fluorine are generally preferred. Fluorine is most preferred when the halogens are substituted on an alkyl or alkoxy group (e.g. CF₃O and CF₃CH₂O).

The compounds of the present invention may contain one or more asymmetric centers and can thus occur as racemates and racemic mixtures, single enantiomers, diastereomeric mixtures and individual diastereomers. The compounds of the present invention have one asymmetric center at the carbon atom marked with an * in formula Ia. Additional asymmetric centers may be present depending upon the nature of the various substituents on the molecule. Each such asymmetric center will independently produce two optical isomers and it is intended that all of the possible optical isomers and diastereomers in mixtures and as pure or partially purified compounds are included within the ambit of this invention. The present invention is meant to comprehend all such isomeric forms of these compounds.

Some of the compounds described herein contain olefinic double bonds, and unless specified otherwise, are meant to include both E and Z geometric isomers.

Some of the compounds described herein may exist as tautomers, which have different points of attachment of hydrogen accompanied by one or more double bond shifts. For example, a ketone and its enol form are keto-enol tautomers. The individual tautomers as well as mixtures thereof are encompassed with compounds of the present invention.

Formula I shows the structure of the class of compounds without preferred stereochemistry. Formula Ia shows the preferred sterochemistry at the carbon atom to which is attached the amino group of the beta amino acid from which these compounds are prepared.

The independent syntheses of these diastereomers or their chromatographic separations may be achieved as known in the art by appropriate modification of the methodology disclosed herein. Their absolute stereochemistry may be determined by the x-ray crystallography of crystalline products or crystalline intermediates which are derivatized, if necessary, with a reagent containing an asymmetric center of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art.

Alternatively, any enantiomer of a compound may be obtained by stereoselective synthesis using optically pure starting materials or reagents of known configuration by methods well known in the art.

It will be understood that, as used herein, references to the compounds of structural formula I are meant to also include the pharmaceutically acceptable salts, and also salts that are not pharmaceutically acceptable when they are used as precursors to the free compounds or their pharmaceutically acceptable salts or in other synthetic manipulations.

The compounds of the present invention may be administered in the form of a pharmaceutically acceptable salt. The term “pharmaceutically acceptable salt” refers to salts prepared from pharmaceutically acceptable non-toxic bases or acids including inorganic or organic bases and inorganic or organic acids. Salts of basic compounds encompassed within the term “pharmaceutically acceptable salt” refer to non-toxic salts of the compounds of this invention which are generally prepared by reacting the free base with a suitable organic or inorganic acid. Representative salts of basic compounds of the present invention include, but are not limited to, the following: acetate, benzenesulfonate, benzoate, bicarbonate, bisulfate, bitartrate, borate, bromide, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, oxalate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate. Furthermore, where the compounds of the invention carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangamous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, cyclic amines, and basic ion-exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

Also, in the case of a carboxylic acid (—COOH) or alcohol group being present in the compounds of the present invention, pharmaceutically acceptable esters of carboxylic acid derivatives, such as methyl, ethyl, or pivaloyloxymethyl, or acyl derivatives of alcohols, such as acetate or maleate, can be employed. Included are those esters and acyl groups known in the art for modifying the solubility or hydrolysis characteristics for use as sustained-release or prodrug formulations.

Solvates, and in particular, the hydrates of the compounds of structural formula I are included in the present invention as well.

Exemplifying the invention is the use of the compounds disclosed in the Examples and herein.

The subject compounds are useful in a method of inhibiting the dipeptidyl peptidase-IV enzyme in a patient such as a mammal in need of such inhibition comprising the administration of an effective amount of the compound. The present invention is directed to the use of the compounds disclosed herein as inhibitors of dipeptidyl peptidase-IV enzyme activity.

In addition to primates, such as humans, a variety of other mammals can be treated according to the method of the present invention. For instance, mammals including, but not limited to, cows, sheep, goats, horses, dogs, cats, guinea pigs, rats or other bovine, ovine, equine, canine, feline, rodent or murine species can be treated. However, the method can also be practiced in other species, such as avian species (e.g., chickens).

The present invention is further directed to a method for the manufacture of a medicament for inhibiting dipeptidyl peptidase-IV enzyme activity in humans and animals comprising combining a compound of the present invention with a pharmaceutically acceptable carrier or diluent.

The subject treated in the present methods is generally a mammal, preferably a human being, male or female, in whom inhibition of dipeptidyl peptidase-IV enzyme activity is desired. The term “therapeutically effective amount” means the amount of the subject compound that will elicit the biological or medical response of a tissue, system, animal or human that is being sought by the researcher, veterinarian, medical doctor or other clinician.

The term “composition” as used herein is intended to encompass a product comprising the specified ingredients in the specified amounts, as well as any product which results, directly or indirectly, from combination of the specified ingredients in the specified amounts. Such term in relation to pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier. By “pharmaceutically acceptable” it is meant the carrier, diluent or excipient must be compatible with the other ingredients of the formulation and not deleterious to the recipient thereof.

The terms “administration of” and or “administering a” compound should be understood to mean providing a compound of the invention or a prodrug of a compound of the invention to the individual in need of treatment.

The utility of the compounds in accordance with the present invention as inhibitors of dipeptidyl peptidase-IV enzyme activity may be demonstrated by methodology known in the art. Inhibition constants are determined as follows. A continuous fluorometric assay is employed with the substrate Gly-Pro-AMC, which is cleaved by DP-IV to release the fluorescent AMC leaving group. The kinetic parameters that describe this reaction are as follows: Kₘ=50 μM; k_{cat}=75 s⁻¹; k_{cat}/Kₘ=1.5×10⁶ M⁻¹s⁻¹. A typical reaction contains approximately 50 pM enzyme, 50 μM Gly-Pro-AMC, and buffer (100 mM HEPES, pH 7.5, 0.1 mg/ml BSA) in a total reaction volume of 100 μl. Liberation of AMC is monitored continuously in a 96-well plate fluorometer using an excitation wavelength of 360 nm and an emission wavelength of 460 nm. Under these conditions, approximately 0.8 μM AMC is produced in 30 minutes at 25 degrees C. The enzyme used in these studies was soluble (transmembrane domain and cytoplasmic extension excluded) human protein produced in a baculovirus expression system (Bac-To-Bac, Gibco BRL). The kinetic constants for hydrolysis of Gly-Pro-AMC and GLP-1 were found to be in accord with literature values for the native enzyme. To measure the dissociation constants for compounds, solutions of inhibitor in DMSO were added to reactions containing enzyme and substrate (final DMSO concentration is 1%). All experiments were conducted at room temperature using the standard reaction conditions described above. To determine the dissociation constants (Kᵢ), reaction rates were fit by non-linear regression to the Michaelis-Menton equation for competitive inhibition. The errors in reproducing the dissociation constants are typically less than two-fold.

In particular, the compounds of the following examples had activity in inhibiting the dipeptidyl peptidase-IV enzyme in the aforementioned assays, generally with an IC₅₀ of less than about 1 μM. Such a result is indicative of the intrinsic activity of the compounds in use as inhibitors the dipeptidyl peptidase-IV enzyme activity.

Dipeptidyl peptidase-IV enzyme (DP-IV) is a cell surface protein that has been implicated in a wide range of biological functions. It has a broad tissue distribution (intestine, kidney, liver, pancreas, placenta, thymus, spleen, epithelial cells, vascular endothelium, lymphoid and myeloid cells, serum), and distinct tissue and cell-type expression levels. DP-IV is identical to the T cell activation marker CD26, and it can cleave a number of immunoregulatory, endocrine, and neurological peptides in vitro. This has suggested a potential role for this peptidase in a variety of disease processes in humans or other species.

Accordingly, the subject compounds are useful in a method for the prevention or treatment of the following diseases, disorders and conditions.

Type II Diabetes and Related Disorders: It is well established that the incretins GLP-1 and GIP are rapidly inactivated in vivo by DP-IV. Studies with DP-IV^{(-/-)}-deficient mice and preliminary clinical trials indicate that DP-IV inhibition increases the steady state concentrations of GLP-1 and GIP, resulting in improved glucose tolerance. By analogy to GLP-1 and GIP, it is likely that other glucagon family peptides involved in glucose regulation are also inactivated by DP-IV (eg. PACAP). Inactivation of these peptides by DP-IV may also play a role in glucose homeostasis.

The DP-IV inhibitors of the present invention therefore have utility in the treatment of type II diabetes and in the treatment and prevention of the numerous conditions that often accompany Type II diabetes, including metabolic syndrome X, reactive hypoglycemia, and diabetic dyslipidemia. Obesity, discussed below, is another condition that is often found with Type II diabetes that may respond to treatment with the compounds of this invention.

The following diseases, disorders and conditions are related to Type 2 diabetes, and therefore may be treated, controlled or in some cases prevented, by treatment with the compounds of this invention: (1) hyperglycemia, (2) low glucose tolerance, (3) insulin resistance, (4) obesity, (5) lipid disorders, (6) dyslipidemia, (7) hyperlipidemia, (8) hypertriglyceridemia, (9) hypercholesterolemia, (10) low HDL levels, (11) high LDL levels, (12) atherosclerosis and its sequelae, (13) vascular restenosis, (14) irritable bowel syndrome, (15) inflammatory bowel disease, including Crohn's disease and ulcerative colitis, (16) other inflammatory conditions, (17) pancreatitis, (18) abdominal obesity, (19) neurodegenerative disease, (20) retinopathy, (21) nephropathy, (22) neuropathy, (23) Syndrome X, (24) ovarian hyperandrogenism (polycystic ovarian syndrome), and other disorders where insulin resistance is a component.

Obesity: DP-IV inhibitors may be useful for the treatment of obesity. This is based on the observed inhibitory effects on food intake and gastric emptying of GLP-1 and GLP-2. Exogenous administration of GLP-1 in humans significantly decreases food intake and slows gastric emptying (Am. J. Physiol., 277: R910-R916 (1999)). ICV administration of GLP-1 in rats and mice also has profound effects on food intake (Nature Medicine, 2: 1254-1258 (1996)). This inhibition of feeding is not observed in GLP-1R^{(-/-)} mice, indicating that these effects are mediated through brain GLP-1 receptors. By analogy to GLP-1, it is likely that GLP-2 is also regulated by DP-IV. ICV administration of GLP-2 also inhibits food intake, analogous to the effects observed with GLP-1 (Nature Medicine, 6: 802-807 (2000)). In addition, studies with DP-IV deficient mice suggest that these animals are resistant to diet-induced obesity and associated pathology (e.g. hyperinsulinonemia).

Growth Hormone Deficiency: DP-IV inhibition may be useful for the treatment of growth hormone deficiency, based on the hypothesis that growth-hormone releasing factor (GRF), a peptide that stimulates release of growth hormone from the anterior pituitary, is cleaved by the DP-IV enzyme in vivo (WO 00/56297). The following data provide evidence that GRF is an endogenous substrate: (1) GRF is efficiently cleaved in vitro to generate the inactive product GRF[3-44] (BBA 1122: 147-153 (1992)); (2) GRF is rapidly degraded in plasma to GRF[3-44]; this is prevented by the DP-IV inhibitor diprotin A; and (3) GRF[3-44] is found in the plasma of a human GRF transgenic pig (J. Clin. Invest., 83:1533-1540 (1989)). Thus DP-IV inhibitors may be useful for the same spectrum of indications which have been considered for growth hormone secretagogues.

Intestinal Injury: The potential for using DP-IV inhibitors for the treatment of intestinal injury is suggested by the results of studies indicating that glucagon-like peptide-2 (GLP-2), a likely endogenous substrate for DP-IV, may exhibit trophic effects on the intestinal epithelium (Regulatory Peptides, 90: 27-32 (2000)). Administration of GLP-2 results in increased small bowel mass in rodents and attenuates intestinal injury in rodent models of colitis and enteritis.

Immunosuppression: DP-IV inhibition may be useful for modulation of the immune response, based upon studies implicating the DP-IV enzyme in T cell activation and in chemokine processing, and efficacy of DP-IV inhibitors in in vivo models of disease. DP-IV has been shown to be identical to CD26, a cell surface marker for activated immune cells. The expression of CD26 is regulated by the differentiation and activation status of immune cells. It is generally accepted that CD26 functions as a co-stimulatory molecule in in vitro models of T cell activation. A number of chemokines contain proline in the penultimate position, presumably to protect them from degradation by non-specific aminopeptidases. Many of these have been shown to be processed in vitro by DP-IV. In several cases (RANTES, LD78-beta, MDC, eotaxin, SDF-1alpha), cleavage results in an altered activity in chemotaxis and signaling assays. Receptor selectivity also appears to be modified in some cases (RANTES). Multiple N-terminally truncated forms of a number of chemokines have been identified in in vitro cell culture systems, including the predicted products of DP-IV hydrolysis.

DP-IV inhibitors have been shown to be efficacious immunosupressants in animal models of transplantation and arthritis. Prodipine (Pro-Pro-diphenyl-phosphonate), an irreversible inhibitor of DP-IV, was shown to double cardiac allograft survival in rats from day 7 to day 14 (Transplantation, 63: 1495-1500 (1997)). DP-IV inhibitors have been tested in collagen and alkyldiamine-induced arthritis in rats and showed a statistically significant attenuation of hind paw swelling in this model [Int. J. Immunopharmacology, 19:15-24 (1997) and Immunopharmacology, 40: 21-26 (1998)]. DP-IV is upregulated in a number of autoimmune diseases including rheumatoid arthritis, multiple sclerosis, Graves' disease, and Hashimoto's thyroiditis (Immunology Today, 20: 367-375 (1999)).

HIV Infection: DP-IV inhibition may be useful for the treatment or prevention of HIV infection or AIDS because a number of chemokines which inhibit HIV cell entry are potential substrates for DP-IV (Immunology Today 20: 367-375 (1999)). In the case of SDF-1alpha, cleavage decreases antiviral activity (PNAS, 95: 6331-6 (1998)). Thus, stabilization of SDF-1alpha through inhibition of DP-IV would be expected to decrease HIV infectivity.

Hematopoiesis: DP-IV inhibition may be useful for the treatment or prevention of hematopiesis because DP-IV may be involved in hematopoiesis. A DP-IV inhibitor, Val-Boro-Pro, stimulated hematopoiesis in a mouse model of cyclophosphamide-induced neutropenia (WO 99/56753).

Neuronal Disorders: DP-IV inhibition may be useful for the treatment or prevention of various neuronal or psychiatric disorders because a number of peptides implicated in a variety of neuronal processes are cleaved in vitro by DP-IV. A DP-IV inhibitor thus may have a therapeutic benefit in the treatment of neuronal disorders. Endomorphin-2, beta-casomorphin, and substance P have all been shown to be in vitro substrates for DP-IV. In all cases, in vitro cleavage is highly efficient, with k_{cat}/Kₘ˜10⁶ M⁻¹s⁻¹ or greater. In an electric shock jump test model of analgesia in rats, a DP-IV inhibitor showed a significant effect that was independent of the presence of exogenous endomorphin-2 (Brain Research, 815: 278-286 (1999)). Neuroprotective and neuroregenerative effects of DP-IV inhibitors were also evidenced by the inhibitors' ability to protect motor neurons from excitotoxic cell death, to protect striatal innervation of dopaminergic neurons when administered concurrently with MPTP, and to promote recovery of striatal innervation density when given in a therapeutic manner following MPTP treatment [see Yong-Q. Wu, et al., “Neuroprotective Effects of Inhibitors of Dipeptidyl Peptidase-IV In Vitro and In Vivo,” Int. Conf. On Dipeptidyl Aminopeptidases: Basic Science and Clinical Applications, Sep. 26-29, 2002 (Berlin, Germany)).

Tumor Invasion and Metastasis: DP-IV inhibition may be useful for the treatment or prevention of tumor invasion and metastasis because an increase or decrease in expression of several ectopeptidases including DP-IV has been observed during the transformation of normal cells to a malignant phenotype (J. Exp. Med., 190: 301-305 (1999)). Up- or down-regulation of these proteins appears to be tissue and cell-type specific. For example, increased CD26/DP-IV expression has been observed on T cell lymphoma, T cell acute lymphoblastic leukemia, cell-derived thyroid carcinomas, basal cell carcinomas, and breast carcinomas. Thus, DP-IV inhibitors may have utility in the treatment of such carcinomas.

Benign Prostatic Hypertrophy: DP-IV inhibition may be useful for the treatment of benign prostatic hypertrophy because increased DP-IV activity was noted in prostate tissue from patients with BPH (Eur. J. Clin. Chem. Clin. Biochem., 30: 333-338 (1992)).

Sperm motility/male contraception: DP-IV inhibition may be useful for the altering sperm motility and for male contraception because in seminal fluid, prostatosomes, prostate derived organelles important for sperm motility, possess very high levels of DP-IV activity (Eur. J. Clin. Chem. Clin. Biochem., 30: 333-338 (1992)).

Gingivitis: DP-IV inhibition may be useful for the treatment of gingivitis because DP-IV activity was found in gingival crevicular fluid and in some studies correlated with periodontal disease severity (Arch. Oral Biol., 37: 167-173 (1992)).

Osteoporosis: DP-IV inhibition may be useful for the treatment or prevention of osteoporosis because GIP receptors are present in osteoblasts.

The compounds of the present invention have utility in treating or preventing one or more of the following conditions or diseases: (1) hyperglycemia, (2) low glucose tolerance, (3) insulin resistance, (4) obesity, (5) lipid disorders, (6) dyslipidemia, (7) hyperlipidemia, (8) hypertriglyceridemia, (9) hypercholesterolemia, (10) low HDL levels, (11) high LDL levels, (12) atherosclerosis and its sequelae, (13) vascular restenosis, (14) irritable bowel syndrome, (15) inflammatory bowel disease, including Crohn's disease and ulcerative colitis, (16) other inflammatory conditions, (17) pancreatitis, (18) abdominal obesity, (19) neurodegenerative disease, (20) retinopathy, (21) nephropathy, (22) neuropathy, (23) Syndrome X, (24) ovarian hyperandrogenism (polycystic ovarian syndrome), (25) Type II diabetes, (26) growth hormone deficiency, (27) neutropenia, (28) neuronal disorders, (29) tumor metastasis, (30) benign prostatic hypertrophy, (32) gingivitis, (33) hypertension, (34) osteoporosis, and other conditions that may be treated or prevented by inhibition of DP-IV.

The subject compounds are further useful in a method for the prevention or treatment of the aforementioned diseases, disorders and conditions in combination with other agents.

The compounds of the present invention may be used in combination with one or more other drugs in the treatment, prevention, suppression or amelioration of diseases or conditions for which compounds of Formula I or the other drugs may have utility, where the combination of the drugs together are safer or more effective than either drug alone. Such other drug(s) may be administered, by a route and in an amount commonly used therefor, contemporaneously or sequentially with a compound of Formula I. When a compound of Formula I is used contemporaneously with one or more other drugs, a pharmaceutical composition in unit dosage form containing such other drugs and the compound of Formula I is preferred. However, the combination therapy may also includes therapies in which the compound of Formula I and one or more other drugs are administered on different overlapping schedules. It is also contemplated that when used in combination with one or more other active ingredients, the compounds of the present invention and the other active ingredients may be used in lower doses than when each is used singly. Accordingly, the pharmaceutical compositions of the present invention include those that contain one or more other active ingredients, in addition to a compound of Formula I.

Examples of other active ingredients that may be administered in combination with a compound of Formula I, and either administered separately or in the same pharmaceutical composition, include, but are not limited to:

(a) other dipeptidyl peptidase IV (DP-IV) inhibitors;

(b) insulin sensitizers including (i) PPARγ agonists such as the glitazones (e.g. troglitazone, pioglitazone, englitazone, MCC-555, rosiglitazone, and the like) and other PPAR ligands, including PPARα/γ dual agonists, such as KRP-297, and PPARα agonists such as fenofibric acid derivatives (gemfibrozil, clofibrate, fenofibrate and bezafibrate), (ii) biguanides such as metformin and phenformin, and (iii) protein tyrosine phosphatase-1B (PTP-1B) inhibitors;

(c) insulin or insulin mimetics;

(d) sulfonylureas and other insulin secretagogues, such as tolbutamide glyburide, glipizide, glimepiride, and meglitinides, such as repaglinide;

(e) α-glucosidase inhibitors (such as acarbose and miglitol);

(f) glucagon receptor antagonists such as those disclosed in WO 98/04528, WO 99/01423, WO 00/39088, and WO 00/69810;

(g) GLP-1, GLP-1 mimetics, and GLP-1 receptor agonists such as those disclosed in WO00/42026 and WO00/59887;

(h) GIP and GIP mimetics such as those disclosed in WO00/58360, and GIP receptor agonists;

(i) PACAP, PACAP mimetics, and PACAP receptor agonists such as those disclosed in WO 01/23420;

(j) cholesterol lowering agents such as (i) HMG-CoA reductase inhibitors (lovastatin, simvastatin, pravastatin, cerivastatin, fluvastatin, atorvastatin, itavastatin, and rosuvastatin, and other statins), (ii) sequestrants (cholestyramine, colestipol, and dialkylaminoalkyl derivatives of a cross-linked dextran), (iii) nicotinyl alcohol, nicotinic acid or a salt thereof, (iv) PPARα agonists such as fenofibric acid derivatives (gemfibrozil, clofibrate, fenofibrate and bezafibrate), (v) PPARα/γ dual agonists, such as KRP-297, (vi) inhibitors of cholesterol absorption, such as beta-sitosterol and ezetimibe, (vii) acyl CoA:cholesterol acyltransferase inhibitors, such as avasimibe, and (viii) anti-oxidants, such as probucol;

(k) PPARδ agonists, such as those disclosed in WO097/28149;

(l) antiobesity compounds such as fenfluramine, dexfenfluramine, phentermine, sibutramine, orlistat, neuropeptide Y5 inhibitors, cannabinoid-1 (CB-1) receptor antagonists/reverse agonists, melanocortin-4 receptor agonists, and β₃ adrenergic receptor agonists;

(m) ileal bile acid transporter inhibitors;

(n) agents intended for use in inflammatory conditions such as aspirin, non-steroidal anti-inflammatory drugs, glucocorticoids, azulfidine, and selective cyclooxygenase-2 inhibitors; and

(o) antihypertensive agents such as ACE inhibitors (enalapril, lisinopril, captopril, quinapril, tandolapril), A-II receptor blockers (losartan, candesartan, irbesartan, valsartan, telmisartan, eprosartan), beta blockers and calcium channel blockers.

The above combinations include combinations of a compound of the present invention not only with one other active compound, but also with two or more other active compounds. Non-limiting examples include combinations of compounds having Formula I with two or more active compounds selected from biguanides, sulfonylureas, HMG-CoA reductase inhibitors, PPAR agonists, PTP-1B inhibitors, other DP-IV inhibitors, and anti-obesity compounds.

Likewise, compounds of the present invention may be used in combination with other drugs that are used in the treatment/prevention/suppression or amelioration of the diseases or conditions for which compounds of the present invention are useful. Such other drugs may be administered, by a route and in an amount commonly used therefor, contemporaneously or sequentially with a compound of the present invention. When a compound of the present invention is used contemporaneously with one or more other drugs, a pharmaceutical composition containing such other drugs in addition to the compound of the present invention is preferred. Accordingly, the pharmaceutical compositions of the present invention include those that also contain one or more other active ingredients, in addition to a compound of the present invention.

The weight ratio of the compound of the present invention to the second active ingredient may be varied and will depend upon the effective dose of each ingredient. Generally, an effective dose of each will be used. Thus, for example, when a compound of the present invention is combined with another agent, the weight ratio of the compound of the present invention to the other agent will generally range from about 1000:1 to about 1:1000, preferably about 200:1 to about 1:200. Combinations of a compound of the present invention and other active ingredients will generally also be within the aforementioned range, but in each case, an effective dose of each active ingredient should be used.

In such combinations the compound of the present invention and other active agents may be administered separately or in conjunction. In addition, the administration of one element may be prior to, concurrent to, or subsequent to the administration of other agent(s).

The compounds of the present invention may be administered by oral, parenteral (e.g., intramuscular, intraperitoneal, intravenous, ICV, intracisternal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration. In addition to the treatment of warm-blooded animals such as mice, rats, horses, cattle, sheep, dogs, cats, monkeys, etc., the compounds of the invention are effective for use in humans.

The pharmaceutical compositions for the administration of the compounds of this invention may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. As used herein, the term “composition” is intended to encompass a product comprising the specified ingredients in the specified amounts, as well as any product which results, directly or indirectly, from combination of the specified ingredients in the specified amounts.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc., containing the compounds of The present invention are employed. (For purposes of this application, topical application shall include mouth washes and gargles.)

The pharmaceutical composition and method of the present invention may further comprise other therapeutically active compounds as noted herein which are usually applied in the treatment of the above mentioned pathological conditions.

In the treatment or prevention of conditions which require inhibition of dipeptidyl peptidase-IV enzyme activity an appropriate dosage level will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are preferably provided in the form of tablets containing 1.0 to 1000 mg of the active ingredient, particularly 1.0, 5.0, 10.0, 15.0. 20.0, 25.0, 50.0, 75.0, 100.0, 150.0, 200.0, 250.0, 300.0, 400.0, 500.0, 600.0, 750.0, 800.0, 900.0, and 1000.0 mg of the active ingredient for the symptomatic adjustment of the dosage to the patient to be treated. The compounds may be administered on a regimen of 1 to 4 times per day, preferably once or twice per day.

When treating or preventing diabetes mellitus and/or hyperglycemia or hypertriglyceridemia or other diseases for which compounds of the present invention are indicated, generally satisfactory results are obtained when the compounds of the present invention are administered at a daily dosage of from about 0.1 mg to about 100 mg per kilogram of animal body weight, preferably given as a single daily dose or in divided doses two to six times a day, or in sustained release form. For most large mammals, the total daily dosage is from about 1.0 mg to about 1000 mg, preferably from about 1 mg to about 50 mg. In the case of a 70 kg adult human, the total daily dose will generally be from about 7 mg to about 350 mg. This dosage regimen may be adjusted to provide the optimal therapeutic response.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Several methods for preparing the compounds of this invention are illustrated in the following Schemes and Examples. Starting materials are made according to procedures known in the art or as illustrated herein.

The compounds of the present invention can be prepared from beta amino acid intermediates such as those of formula II and substituted heterocyclic intermediates such as those of formula III, using standard peptide coupling conditions followed by deprotection. The preparation of these intermediates is described in the following schemes. 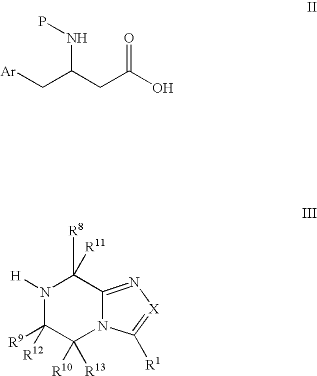
where Ar, X, R¹, R⁸, R⁹, R¹⁰, R¹¹, R¹² and R¹³ are as defined above and P is a suitable nitrogen protecting group such as tert-butoxycarbonyl, benzyloxycarbonyl, and 9-fluorenylmethoxycarbonyl.

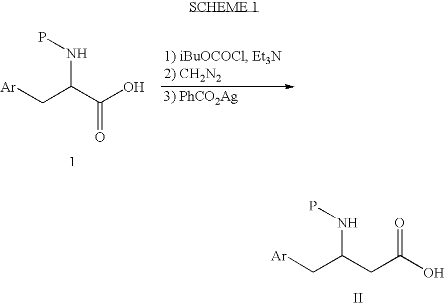

Compounds of formula II are commercially available, known in the literature or may be conveniently prepared by a variety of methods familiar to those skilled in the art. One common route is illustrated in Scheme 1. Protected alpha-amino acid 1, which may be commercially available or readily prepared from the corresponding amino acid by protection using, for example, di-tert-butyl dicarbonate (for P=BOC), carbobenzyloxy chloride (for P=Cbz), or N-(9-fluorenylmethoxycarbonyloxy)succinimide (for P=Fmoc), is treated with isobutyl chloroformate and a base such as triethylamine or N,N-diisopropylethylamine (DIEA), followed by diazomethane. The resultant diazoketone is then treated with silver benzoate in a solvent such as methanol or aqueous dioxane which may be subjected to sonication following the procedure of Sewald et al., Synthesis, 837 (1997) in order to provide the beta amino acid II. As will be understood by those skilled in the art, for the preparation of enantiomerically pure beta amino acids II, enantiomerically pure alpha amino acids 1 may be used. Alternate routes to the protected beta-amino acid intermediates II can be found in the following reviews: E. Juaristi, Enantioselective Synthesis of β-Amino Acids, Ed., Wiley-VCH, New York: 1997; Juaristi et al., Aldrichimica Acta, 27: 3 (1994); and Cole et al., Tetrahedron, 32: 9517 (1994).

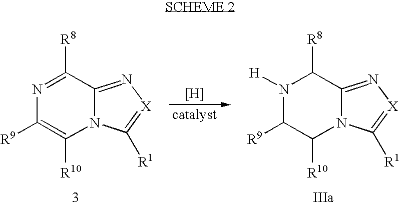

Compounds III are commercially available, known in the literature or may be conveniently prepared by a variety of methods familiar to those skilled in the art. One convenient method for the preparation of compounds IIIa, wherein R¹¹, R¹² and R¹³ are hydrogen, is shown in Scheme 2. Unsaturated derivative 3 is reduced, for example, by treatment with hydrogen gas and a catalyst such as palladium on carbon or platinum oxide in a solvent such as methanol or ethanol to provide Compound IIIa.

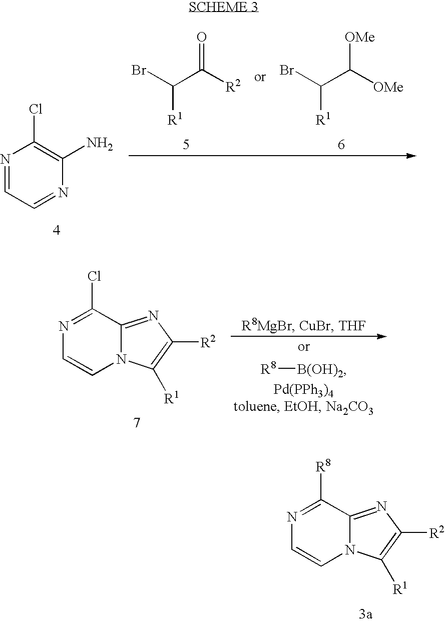

Intermediates 3, from Scheme 2, are themselves commercially available, known in the literature or may be conveniently prepared by a variety of methods familiar to those skilled in the art. One such method when X is CR² and R⁹ and R¹⁰ are H is illustrated in Scheme 3. Aminopyrazine 4 is treated with a 2-haloketone such as 2-bromoketone 5 in a solvent such as methanol or ethanol to provide intermediate 7. Alternatively, for the preparation of intermediate 7 where R² is H, 2-bromo-dimethylacetal 6 and a catalytic amount of acid such as hydrochloric acid may be employed instead of intermediate 5. The conversion of 4 to 7 may also be carried out in two steps. First, 4 and an appropriate bromide 5 are heated, conveniently in a solvent such as dioxane at 50° C. for 16 h. Then, solvent is removed, the residue treated with isopropanol, and the mixture heated at reflux for approximately 2 h. Intermediate 7 is converted to 3a by treatment with a Grignard reagent in the presence of copper bromide according to the literature procedure detailed in J. Org. Chem., 52: 3847 (1987) or by palladium-catalyzed Suzuki coupling with a boronic acid.

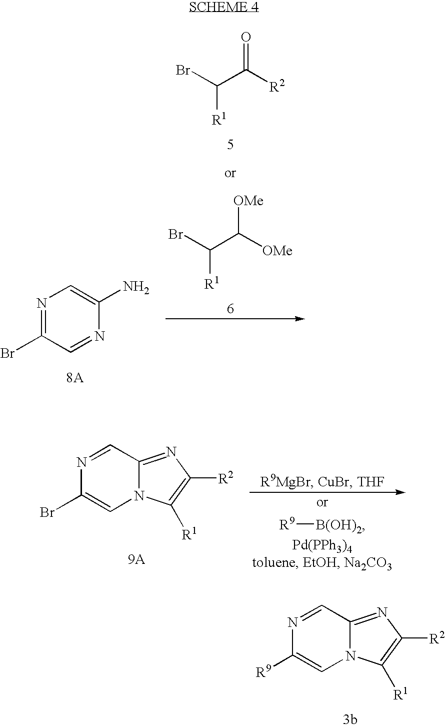

Intermediates 3b, wherein X is CR² and R⁸ and R¹⁰ are H, may be prepared as described above for Scheme 3 starting with aminopyrazine 8A, as illustrated in Scheme 4.

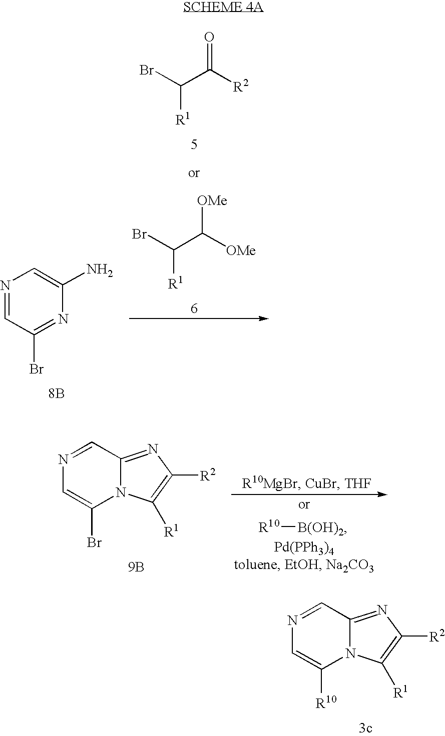

Intermediates 3c, wherein X is CR² and R⁸ and R⁹ are H, may be prepared as described above for Scheme 3 starting with aminopyrazine 8B, as illustrated in Scheme 4A.

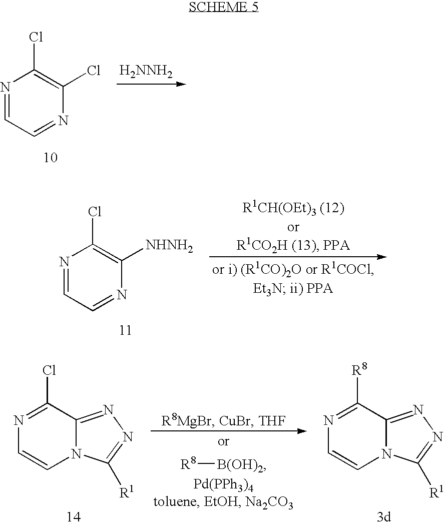

An alternate route for the preparation of Compound 3d wherein X is N and R⁹ and R¹⁰ are H is illustrated in Scheme 5. Dichloropyrazine 10 is treated with hydrazine to provide hydrazinochloropyrazine 11. Compound 11 may be condensed with either an orthoester such as triethyl orthoester 12 or with a carboxylic acid 13 in polyphosphoric acid (PPA) at elevated temperatures to give 14. Alternatively, the hydrazine 11 may be acylated, for example, by treatment with an acid chloride or anhydride in the presence of a base such as triethylamine, and the resultant hydrazide cyclized to 14 by heating in polyphosphoric acid. Displacement of the halide using a Grignard reagent or Suzuki coupling as described above provides 3d.

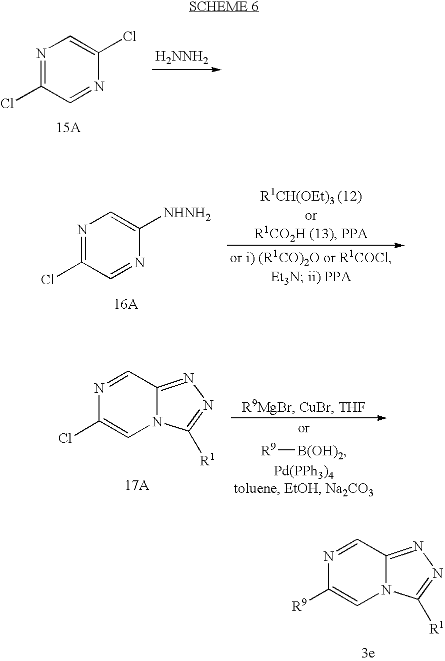

Intermediates 3e, wherein X is N and R⁸ and R¹⁰ are H, may be prepared as described above for Scheme 5 starting with dichloropyrazine 15A, as illustrated in Scheme 6.

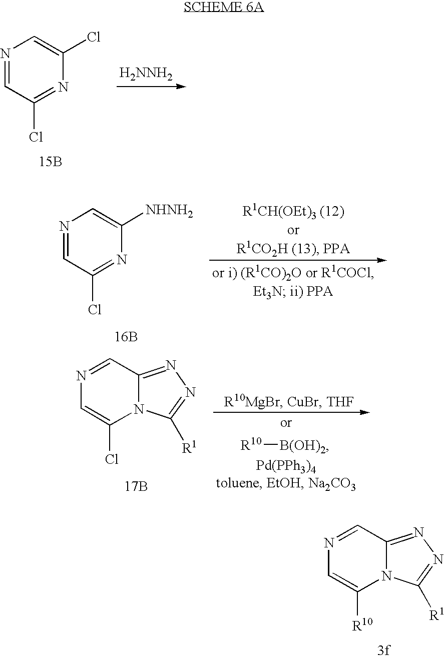

Intermediates 3f, wherein X is N and R⁸ and R⁹ are H, may be prepared as described above for Scheme 5 starting with dichloropyrazine 15B, as illustrated in Scheme 6A.

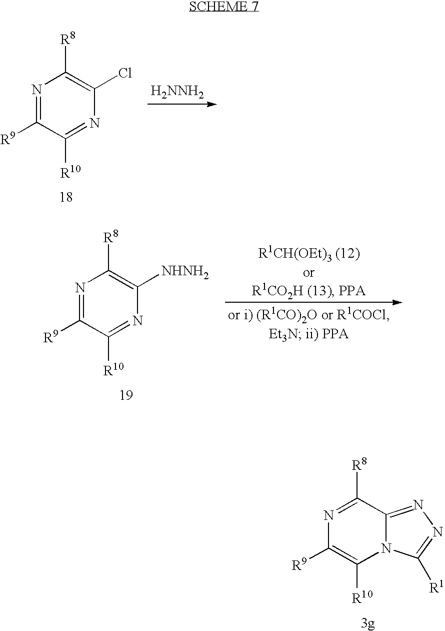

Intermediates 3g, wherein X is N, may be prepared from chloropyrazine 18 as illustrated in Scheme 7. Chloropyrazine 18, which is commercially available, known in the literature or may be conveniently prepared by a variety of methods familiar to those skilled in the art, is treated with hydrazine to provide hydrazinopyrazine 19, Intermediate 19 may be condensed with either an orthoester such as triethyl orthoester 12 or with a carboxylic acid 13 in polyphosphoric acid at elevated temperatures to give 3g. Alternatively, the hydrazine 19 may be acylated, for example, by treatment with an acid chloride or anhydride in the presence of a base such as triethylamine, and the resultant hydrazide cyclized to 3g by heating in polyphosphoric acid.

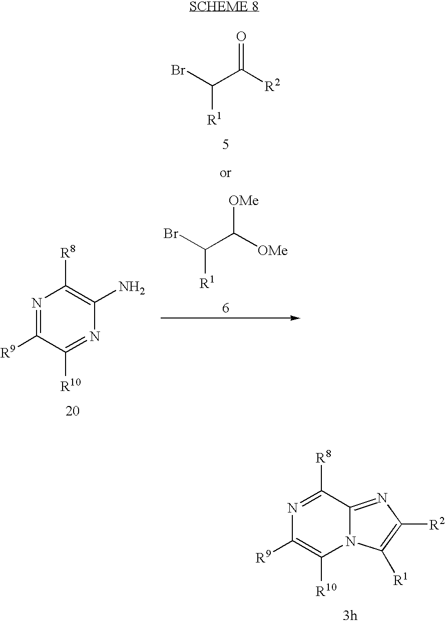

Intermediates 3h, wherein X is CR², may also be prepared as illustrated in Scheme 8. Aminopyrazine 20, which is commercially available, known in the literature or may be conveniently prepared by a variety of methods familiar to those skilled in the art, is treated with a 2-haloketone such as 2-bromoketone 5 in a solvent such as methanol or ethanol to provide intermediate 3h. Alternatively, for the preparation of intermediate 3h where R² is H, 2-bromo-dimethylacetal 6 and a catalytic amount of acid such as hydrochloric acid may be employed instead of intermediate 5. The conversion of 20 to 3h may also be carried out in two steps. First, aminopyrazine 20 and an appropriate bromide 5 are heated, conveniently in a solvent such as dioxane at 50° C. for 16 h. Then, solvent is removed, the residue treated with isopropanol, and the mixture heated at reflux for approximately 2 h to provide 3h.

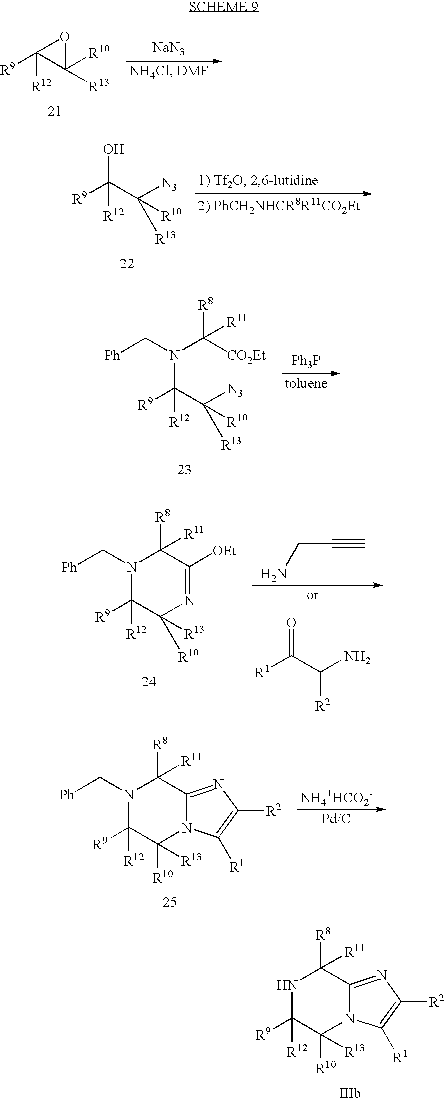

An alternate method for the preparation of Intermediate IIIb, wherein X is C—R² is illustrated in Scheme 9. Ring opening of epoxide 21, which is commercially available, known in the literature or may be conveniently prepared by a variety of methods familiar to those skilled in the art, with azide, conveniently using sodium azide in the presence of ammonium chloride in DMF, gives azido alcohol 22. The alcohol is converted to the corresponding triflate, and treated with an N-benzyl alpha-amino acid ester, such as an ethyl ester, to provide amino ester 23. Aza-Wittig reaction affords iminoether 24. Treatment of 24 with 3-aminopropyne according to the procedure of Maffrand et al., Eur. J. Med. Chem., 10: 528 (1975) gives tetrahydroimidazopyrazine 25, wherein R¹ is Me and R² is H, which may be deprotected using ammonium formate in the presence of a palladium catalyst to give Intermediate IIIb. Alternatively, iminoether 24 may be converted to tetrahydroimidazopyrazine 25 by treatment with an alpha-aminoketone, following procedures outlined in Claxton et al., J. Med. Chem., 17: 364 (1974).

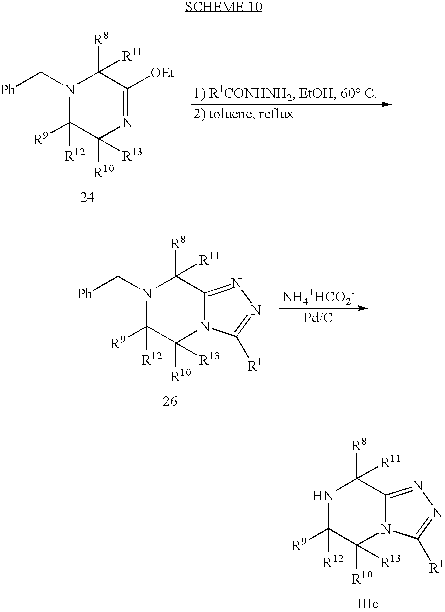

An alternate route to the preparation of Intermediate IIIc, wherein X is N, is illustrated in Scheme 10. Iminoether 24, from Scheme 9, is treated with a hydrazide in, for example, ethanol at 60° C., followed by heating in toluene at reflux, to give triazolopiperazine 26. Deprotection, for example, using ammonium formate in the presence of a palladium catalyst, affords Intermediate IIIc.

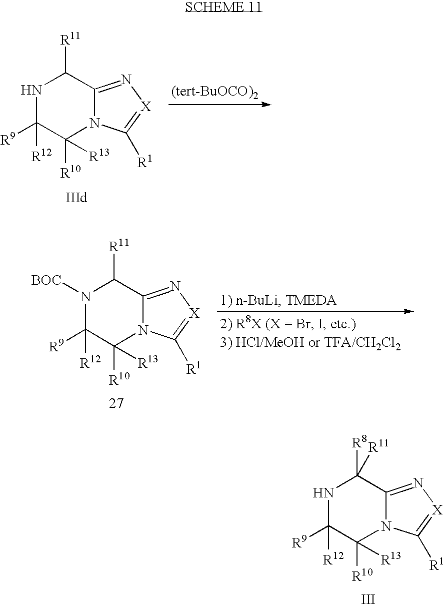

Compounds III may be prepared from intermediate IIId, wherein R⁸ is H, as illustrated in Scheme 11. Intermediate IIId is protected with a nitrogen protecting group, for example, a BOC group by treatment with di-tert-butyldicarbonate. N-BOC derivative 27 is deprotonated with strong base such as n-butyl lithium in the presence of TMEDA and treated with an alkylating agent such as an alkyl halide. Deprotection under acidic conditions provides compound III.

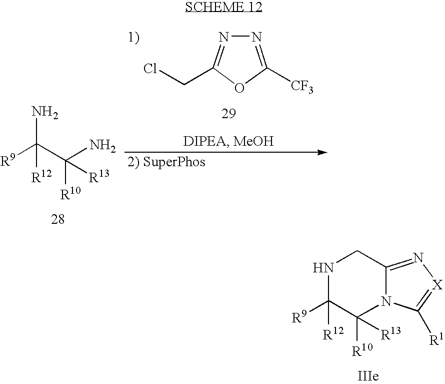

Compounds IIIe, wherein R⁸ and R¹¹ are hydrogen, may be prepared as shown in Scheme 12. Diamine 28 is treated with oxadiazole 29 in a solvent such as methanol, conveniently at 0° C. in the presence of a base such as N,N-diisopropylethylamine. Following removal of methanol, the intermediate mixture may be heated in superphosphoric acid to provide the desired compounds IIIe.

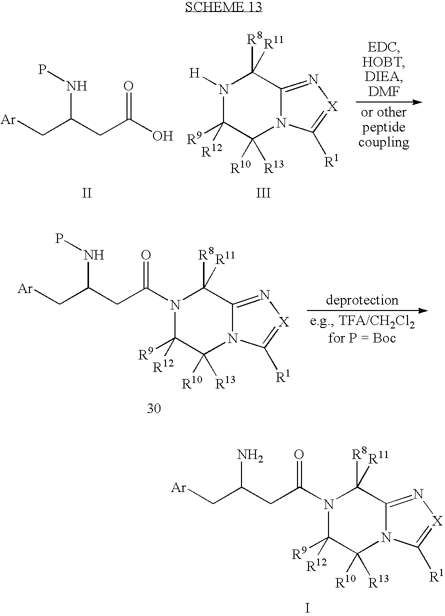

Intermediates II and III are coupled under standard peptide coupling conditions, for example, using 1-ethyl-3-(3-dimethylaminopropyl)carbodiimide and 1-hydroxybenzotriazole (EDC/HOBT) or O-(7-azabenzotriazol-1-yl)-N,N,N′,N′-tetramethyluronium hexafluorophosphate and 1-hydroxy-7-azabenzotriazole (HATU/HOAT) in a solvent such as N,N-dimethylformamide (DMF) or dichloromethane for 3 to 48 hours at ambient temperature to provide Intermediate 30 as shown in Scheme 13. In some cases, Intermediate III may be a salt, such as a hydrochloride or trifluoroacetic acid salt, and in these cases it is convenient to add a base, generally N,N-diisopropylethylamine, to the coupling reaction. The protecting group is then removed with, for example, trifluoroacetic acid or methanolic hydrogen chloride in the case of Boc to give the desired amine I. The product is purified from unwanted side products, if necessary, by recrystallization, trituration, preparative thin layer chromatography, flash chromatography on silica gel, such as with a Biotage® apparatus, or HPLC. Compounds that are purified by HPLC may be isolated as the corresponding salt. Purification of intermediates is achieved in the same manner.

In some cases the product I, prepared as described in Scheme 13, may be further modified, for example, by manipulation of substituents on Ar, R¹, R², R⁸-R¹³. These manipulations may include, but are not limited to, reduction, oxidation, alkylation, acylation, and hydrolysis reactions that are commonly known to those skilled in the art.

In some cases intermediates described in the above schemes may be further modified before the sequences are completed, for example, by manipulation of substituents on Ar, R¹, R², R⁸-R¹³. These manipulations may include, but are not limited to, reduction, oxidation, alkylation, acylation, and hydrolysis reactions that are commonly known to those skilled in the art.

In some cases the order of carrying out the foregoing reaction schemes may be varied to facilitate the reaction or to avoid unwanted reaction products. The following examples are provided so that the invention might be more fully understood. These examples are illustrative only and should not be construed as limiting the invention in any way.

### Intermediate 1

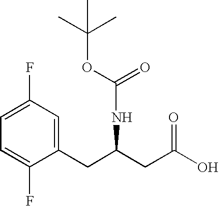

### (3R)-3-[(tert-Butoxycarbonyl)amino]-4-(2,5-difluorophenyl)butanoic acid

### Step A: (R,S)—N-(tert-Butoxycarbonyl)-2,5-difluorophenylalanine

To a solution of 0.5 g (2.49 mmol) of 2,5-difluoro-DL-phenylalanine in 5 mL of tert-butanol were added sequentially 1.5 mL of 2N aqueous sodium hydroxide solution and 543 mg of di-tert-butyl dicarbonate. The reaction was stirred at ambient temperature for 16 h and diluted with ethyl acetate. The organic phase was washed sequentially with 1N hydrochloric acid and brine, dried over magnesium sulfate and concentrated in vacuo. The crude material was purified by flash chromatography (silica gel, 97:2:1 dichloromethane:methanol:acetic acid) to afford the title compound. LC/MS 302 (M+1).

### Step B: (R,S)-3-[(tert-Butoxycarbonyl)amino]-1-diazo-4-(2,5-difluoro-phenyl)butan-2-one

To a solution of 2.23 g (7.4 mmol) of (R,S)—N-(tert-butoxycarbonyl)-2,5-difluorophenylalanine in 100 mL of diethyl ether at 0° C. were added sequentially 1.37 mL (8.1 mmol) of triethylamine and 0.931 mL (7.5 mmol) of isobutyl chloroformate and the reaction was stirred at this temperature for 15 min. A cooled ethereal solution of diazomethane was then added until the yellow color persisted and stirring was continued for a further 16 h. The excess diazomethane was quenched by dropwise addition of acetic acid, and the reaction was diluted with ethyl acetate and washed sequentially with 5% hydrochloric acid, saturated aqueous sodium bicarbonate solution and brine, dried over magnesium sulfate and concentrated in vacuo. Purification by flash chromatography (silica gel, 4:1 hexane:ethyl acetate) afforded the diazoketone.

¹H NMR (500 MHz, CDCl₃): δ 7.03-6.95 (m, 1H), 6.95-6.88 (m, 2H), 5.43 (bs, 1H), 5.18 (bs, 1H), 4.45 (bs, 1H), 3.19-3.12 (m, 1H), 2.97-2.80 (m, 1H), 1.38 (s, 9H).

### Step C: (3R)-3-[(tert-Butoxycarbonyl)amino]-4-(2,5-difluorophenyl)butanoic acid

To a solution of 2.14 g (6.58 mmol) of (R,S)-3-[(tert-butoxycarbonyl)-amino]-1-diazo-4-(2,5-difluorophenyl)butan-2-one dissolved in 100 mL of methanol at −30 ° C. were added sequentially 3.3 mL (19 mmol) of N,N-diisopropylethylamine and 302 mg (1.32 mmol) of silver benzoate. The reaction was stirred for 90 min before diluting with ethyl acetate and washing sequentially with 2N hydrochloric acid, saturated aqueous sodium bicarbonate, and brine. The organic phase was dried over magnesium sulfate, concentrated in vacuo and the enantiomers were separated by preparative chiral HPLC (Chiralpak AD column, 5% ethanol in hexanes) to give the desired (R)-enantiomer, which eluted first. This material was dissolved in 50 mL of a mixture of tetrahydrofuran:methanol:1N aqueous lithium hydroxide (3:1:1) and stirred at 50° C. for 4 h. The reaction was cooled, acidified with 5% dilute hydrochloric acid and extracted with ethyl acetate. The combined organic phases were washed with brine, dried over magnesium sulfate and concentrated in vacuo to give the title compound as a white foamy solid.

¹H NMR (500 MHz, CDCl₃): δ 7.21 (m, 1H), 6.98 (m, 2H), 6.10 (bs, 1H), 5.05 (m,1H), 4.21 (m, 1H), 2.98 (m, 2H), 2.60 (m, 2H), 1.38 (s, 9H).

### Intermediate 2

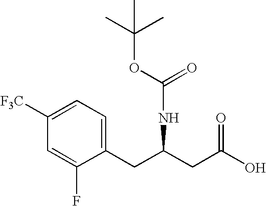

### (3R)-3-[(tert-Butoxycarbonyl)amino]-4-[2-fluoro-4-(trifluoromethyl)phenyl]-butanoic acid

### Step A: (2R,5S)-2,5-Dihydro-3,6-dimethoxy-2-(2′-fluoro-4′-(trifluoromethyl)benzyl)-5-isopropylpyrazine

To a solution of 3.32 g (18 mmol) of commercially available (2S)-2,5-dihydro-3,6-dimethoxy-2-isopropylpyrazine in 100 mL of tetrahydrofuran at −70 ° C. was added 12 mL (19 mmol) of a 1.6M solution of butyllithium in hexanes. After stirring at this temperature for 20 min, 5 g (19.5 mmol) of 2-fluoro-4-trifluoromethylbenzyl bromide in 20 mL of tetrahydrofuran was added and stirring was continued for 3 h before warming the reaction to ambient temperature. The reaction was quenched with water, concentrated in vacuo, and extracted with ethyl acetate. The combined organic phase was washed with brine, dried, and concentrated in vacuo. Purification by flash chromatography (silica gel, 0-5% ethyl acetate in hexanes) afforded the title compound.

¹H NMR (500 MHz, CDCl₃): δ 7.33-7.25 (m, 3H), 4.35-4.31 (m, 1H), 3.75 (s, 3H), 3.65 (s, 3H), 3.60 (t, 1H, J=3.4 Hz), 3.33 (dd, 1H, J=4.6, 13.5 Hz), 3.03 (dd, 1H, J=7, 13.5 Hz), 2.25-2.15 (m, 1H), 1.0 (d, 3H, J=7 Hz), 0.66 (d, 3H, J=7 Hz).

### Step B: (R)—N-(tert-Butoxycarbonyl)-2-fluoro-4-trifluoromethyl-phenylalanine methyl ester

To a solution of 5.5 g (15 mmol) of (2R,5S)-2,5-dihydro-3,6-dimethoxy-2-(2′-fluoro-4′-(trifluoromethyl)benzyl)-5-isopropylpyrazine in 50 mL of a mixture of acetonitrile:dichloromethane (10:1) was added 80 mL of 1N aqueous trifluoroacetic acid. The reaction was stirred for 6 h and the organic solvents were removed in vacuo. Sodium carbonate was added until the solution was basic (>pH 8), and then the reaction was diluted with 100 mL of tetrahydrofuran and 10 g (46 mmol) of di-tert-butyl dicarbonate was added. The resulting slurry was stirred for 16 h, concentrated in vacuo, and extracted with ethyl acetate. The combined organic phase was washed with brine, dried, and concentrated in vacuo. Purification by flash chromatography (silica gel, 20% ethyl acetate in hexanes) afforded the title compound.

¹H NMR (500 MHz, CDCl₃): δ 7.38-7.28 (m, 3H), 5.10 (bd, 1H), 4.65-3.98 (m, 1H), 3.76 (s, 3H), 3.32-3.25 (m, 1H), 3.13-3.05 (m, 1H), 1.40 (s, 9H).

### Step C: (R)—N-(tert-Butoxycarbonyl)-2-fluoro-4-(trifluoromethyl)phenylalanine

A solution of 5.1 g (14 mmol) of (R,S)—N-(tert-butoxycarbonyl)-2-fluoro-4-(trifluoromethyl)phenylalanine methyl ester in 350 mL of a mixture of tetrahydrofuran:methanol:1N lithium hydroxide (3:1:1) was stirred at 50° C. for 4 h. The reaction was cooled, acidified with 5% hydrochloric acid and extracted with ethyl acetate. The combined organic phases were washed with brine, dried over magnesium sulfate and concentrated in vacuo to give the title compound.

¹H NMR (500 MHz, CD₃OD): δ 7.45-7.38 (m, 3H), 4.44-4.40 (m, 1H), 3.38-3.33 (m, 1H), 2.98 (dd, 1H, J=9.6, 13.5 Hz), 1.44 (s, 9H).

### Step D: (3R)-3-[(tert-Butoxycarbonyl)amino]-4-[2-fluoro-4-(trifluoromethyl)-phenyl]-butanoic acid

To a solution of 3.4 g (9.7 mmol) of the product from Step C in 60 mL of tetrahydrofuran at 0° C. were added sequentially 2.3 mL (13 mmol) of N,N-diisopropylethylamine and 1.7 mL (13 mmol) of isobutyl chloroformate and the reaction was stirred at this temperature for 30 min. A cooled ethereal solution of diazomethane was then added until the yellow color persisted and stirring was continued for a further 16 h. The excess diazomethane was quenched by dropwise addition of acetic acid, and the reaction was diluted with ethyl acetate and washed sequentially with 5% hydrochloric acid, saturated aqueous sodium bicarbonate solution and brine, dried over magnesium sulfate and concentrated in vacuo. Purification by flash chromatography (silica gel, 9:1 hexane:ethyl acetate) afforded 0.5 g of diazoketone. To a solution of 0.5 g (1.33 mmol) of the diazoketone dissolved in 100 mL of methanol at 0° C. were added sequentially 0.7 mL (4 mmol) of N,N-diisopropylethylamine and 32 mg (0.13 mmol) of silver benzoate. The reaction was stirred for 2 h before diluting with ethyl acetate and washing sequentially with 2N hydrochloric acid, saturated aqueous sodium bicarbonate, and brine. The organic phase was dried over magnesium sulfate, concentrated in vacuo and dissolved in 50 mL of a mixture of tetrahydrofuran:methanol:1N aqueous lithium hydroxide (3:1:1) and stirred at 50° C. for 3 h. The reaction was cooled, acidified with 5% hydrochloric acid and extracted with ethyl acetate. The combined organic phases were washed with brine, dried over magnesium sulfate and concentrated in vacuo to give the title compound as a white foamy solid.

¹H NMR (500 MHz, CD₃OD): δ 7.47-7.33 (m, 3H), 4.88 (bs, 1H), 4.26-3.98 (m, 1H), 3.06-3.01 (m, 1H), 2.83-2.77 (m, 1H), 2.58-2.50 (m, 2H), 1.29 (s, 9H).

### Intermediate 3

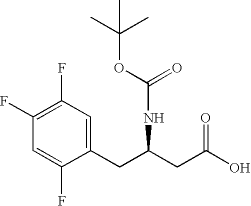

### (3R)-3-[(tert-Butoxycarbonyl)amino]-4-(2,4,5-trifluorophenyl)butanoic acid

### Step A: (2S, 5R)-2,5-Dihydro-3,6-dimethoxy-2-isopropyl-5-(2′,4′,5′trifluorobenzyl)-pyrazine

The title compound (3.81 g) was prepared from 3.42 g (18.5 mmol) of (2S)-2,5-dihydro-3,6-dimethoxy-2-isopropylpyrazine and 5 g (22.3 mmol) of 2,4,5-trifluorobenzyl bromide using the procedure described for Intermediate 2, Step A.

¹H NMR (500 MD, CDCl₃): δ 7.01 (m, 1H), 6.85 (m, 1H), 4.22 (m, 1H), 3.78 (m, 3H), 3.64 (m, 3H), 3.61 (m, 1H), 3.20 (m, 1H), 2.98 (m, 1H), 2.20 (m, 1H), 0.99 (d, 3H, J=8 Hz), 0.62 (d, 3H, J=8 Hz).

### Step B: (R)—N-(tert-Butoxycarbonyl)-2,4,5-trifluorophenylalanine methyl ester

To a solution of 3.81 g (11.6 mmol) of (2S,5R)-2,5-dihydro-3,6-dimethoxy-2-isopropyl-5-(2′,4′,5′trifluorobenzyl)pyrazine in 20 mL of acetonitrile was added 20 mL of 2N hydrochloric acid. The reaction was stirred for 72 h and concentrated in vacuo. The residue was dissolved in 30 mL of dichloromethane and 10 mL (72 mmol) of triethylamine and 9.68 g (44.8 mmol) of di-tert-butyl dicarbonate were added. The reaction was stirred for 16 h, diluted with ethyl acetate and washed sequentially with 1N hydrochloric acid and brine. The organic phase was dried over sodium sulfate, concentrated in vacuo and purified by flash chromatography (silica gel, 9:1 hexanes:ethyl acetate) to afford the title compound.

¹H NMR (500 MHz, CDCl₃): δ 6.99 (m, 1H), 6.94 (m, 1H), 5.08 (m, 1H), 4.58 (m, 1H), 3.78 (m, 3H), 3.19 (m, 1H), 3.01 (m, 1H), 1.41 (s, 9H).

### Step C: (R)—N-(tert-Butoxycarbonyl)-2,4,5-trifluorophenylalanine

The title compound was prepared from 2.41 g (7.5 mmol) of (R)—N-(tert-butoxycarbonyl)-2,4,5-trifluorophenylalanine methyl ester using the procedure described for Intermediate 2, Step C.

LC-MS 220.9 (M+1-BOC).

### Step D: (3R)-3-[(tert-Butoxycarbonyl)amino]-4-(2,4,5-trifluorophenyl)-butanoic acid

To a solution of 0.37 g (1.16 mmol) of (R)—N-(tert-butoxycarbonyl)-2,4,5-trifluorophenylalanine in 10 mL of diethyl ether at −20° C. were added sequentially 0.193 mL (1.3 mmol) of triethylamine and 0.18 mL (1.3 mmol) of isobutyl chloroformate, and the reaction was stirred at this temperature for 15 min. A cooled ethereal solution of diazomethane was then added until the yellow color persisted and stirring was continued for a further 1 h. The excess diazomethane was quenched by dropwise addition of acetic acid, and the reaction was diluted with ethyl acetate and washed sequentially with saturated aqueous sodium bicarbonate solution and brine, dried over magnesium sulfate and concentrated in vacuo. Purification by flash chromatography (silica gel, 3:1 hexane:ethyl acetate) afforded 0.36 g of diazoketone. To a solution of 0.35 g (1.15 mmol) of the diazoketone dissolved in 12 mL of 1,4-dioxane:water (5:1) was added 26 mg (0.113 mmol) of silver benzoate. The resultant solution was sonicated for 2 h before diluting with ethyl acetate and washing sequentially with 1N hydrochloric acid and brine, drying over magnesium sulfate and concentrating in vacuo. Purification by flash chromatography (silica gel, 97:2:1 dichloromethane:methanol:acetic acid) afforded the title compound.

¹H NMR (500 MHz, CDCl₃): δ 7.06 (m, 1H), 6.95 (m, 1H), 5.06 (bs, 1H), 4.18 (m, 1H), 2.98 (m, 2H), 2.61 (m, 2H), 1.39 (s, 9H).

### Intermediate 4

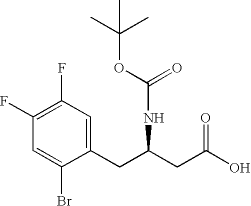

### (3R)-4-(2-Bromo-4,5-difluorophenyl)-3-[(tert-butoxycarbonyl)amino]-butanoic acid

To a solution of 2.4 g (10 mmol) of 2-bromo-4,5-difluorobenzoic acid [prepared according to the procedure of Braish et al., Syn. Comm., 3067-3074 (1992)] in 75 mL of tetrahydrofuran was added 2.43 g (15 mmol) of 1,1′-carbonyldiimidazole. The solution was heated under reflux for 3.5 h, cooled to ambient temperature and 0.38 g (10 mmol) of sodium borohydride in 15 mL of water was added. The reaction was stirred for 10 min and partitioned between ethyl acetate and 10% aqueous sodium bicarbonate solution. The organic layer was washed twice with warm water, brine, dried over magnesium sulfate, and concentrated in vacuo. Purification by flash chromatography (silica gel, 4:1 hexane:ethyl acetate) afforded 1.9 g of 2-bromo-4,5-difluorobenzyl alcohol. To a solution of 1.9 g (8.4 mmol) of 2-bromo-4,5-difluorobenzyl alcohol in 30 mL of dichloromethane at 0° C. was added 3.4 g (10 mmol) of carbon tetrabromide and 2.7 g (10 mmol) of triphenylphosphine. The reaction was stirred for 2 h at this temperature, the solvent was removed in vacuo and the residue stirred with 100 mL of diethyl ether. The solution was filtered, concentrated in vacuo, and purified by flash chromatography (silica gel, 20:1 hexane:ethyl acetate) to afford 2.9 g of 2-bromo-4,5-difluorobenzyl bromide contaminated with carbon tetrabromide which was used without further purification. Using the procedures outlined for the preparation of Intermediates 1-3, the benzyl bromide derivative was converted to the title compound.

LC/MS 394 and 396 (M+1).

Essentially following the procedures outlined for the preparation of Intermediates 1-4, the Intermediates in Table 1 were prepared.

TABLE 1

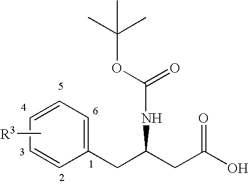

| Inter- | | |
|---|---|---|
| mediate | R³ | Selected ¹H NMR data (CD₃OD) |
| 5 | 2-F,4-Cl,5-F | 7.11 (dd, 1H, J=8.9, 6.4Hz), 7.03 (dd, |
| | | 1H, J=9.0, 6.6) |
| 6 | 2-F,5-Cl | 7.27 (dd, 1H, J=6.4, 2.5Hz), 7.21 (m, |
| | | 1H), 7.03 (t, 1H, J=9.2Hz) |
| 7 | 2-Me,5-Cl | 7.16 (d, 1H, J=1.8Hz), 7.11-7.07 (m, |
| | | 2H), 2.34 (s, 3H) |
| 8 | 2-Cl,5-Cl | 7.34 (d, 1H, J=9.0), 7.33 (d, 1H, J=2.1 |
| | | Hz), 7.21 (dd, 1H, J=8.5, 2.5Hz) |
| 9 | 2-F,3-Cl,6-F | 7.35 (td, 1H, J=8.5, 5.8Hz), 6.95 (t, |
| | | 1H, J=8.5Hz) |
| 10 | 3-Cl,4-F | 7.33 (d, 1H, J=6.9Hz), 7.19-7.11 (m, |
| | | 2H) |
| 11 | 2-F,3-F,6-F | 7.18-7.12 (m, 1H), 6.91 (m, 1H) |
| 12 | 2-F,4-F,6-F | 6.81 (t, 2H, J=8.4Hz) |
| 13 | 2-OCH₂Ph,5-F | 7.49 (d, 2H, J=7.6Hz), 7.38 (t, 2H, J= |
| | | 7.3Hz), 7.30 (t, 1H, J=7.3Hz), 6.96- |
| | | 6.89 (m, 3H), 5.11 (d, 1H, J=11.7Hz), |
| | | 5.08 (d, 1H, J=11.9Hz) |

### EXAMPLE 1

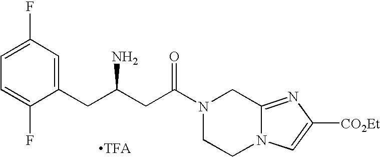

### Ethyl 7-[(3R)-3-amino-4-(2,5-difluorophenyl)butanoyl]-5,6,7,8-tetrahydroimidazo[1,2-a]pyrazine-2-carboxylic acid, trifluoroacetic acid salt

### Step A: Ethyl imidazo[1,2-a]pyrazine-2-carboxylate

To a solution of 2-aminopyrazine (1.0 g, 10.5 mmol) in dioxane (25 mL) was added ethyl 3-bromo-2-ketopropionate (2.0 g, 10.5 mmol). The reaction was stirred at 50° C. for 16 h. The mixture was filtered and the solid was washed with two portions of ethyl acetate. The solid was heated in 35 mL of isopropanol at reflux temperature for 4 h. The reaction mixture was concentrated and partitioned between ethyl acetate and saturated aqueous sodium bicarbonate. The aqueous phase was extracted with three portions of ethyl acetate. The combined organics were washed with brine, dried over magnesium sulfate, and concentrated. Purification by chromatography (Biotage system, silica gel, ethyl acetate then 10% methanol/ethyl acetate) gave the title compound as a solid.

### Step B: Ethel 5,6,7,8-tetrahydroimidazo[1,2-a]pyrazine-2-carboxylate

A mixture of 1.9 g of ethyl imidazo[1,2-a]pyrazine-2-carboxylate (Step A) and 280 mg of 10% palladium on carbon was stirred under an atmosphere of hydrogen overnight. The mixture was filtered through a pad of Celite and the filtrate concentrated. Purification by flash chromatography on a Biotage® system (silica gel, eluting with 50% ethyl acetate/hexane, ethyl acetate, 10% methanol/ethyl acetate and 80:15:1 chloroform/methanol/ammonium hydroxide) gave the title compound.

### Step C: Ethyl 7-[(3R)-3-[(tert-butoxycarbonyl)amino]-4-(2,5-difluorophenyl)butanoyl]-5,6,7,8-tetrahydroimidazo[1,2-a]pyrazine-2-carboxylate

To a solution of 150 mg (0.768 mmol) of ethyl 5,6,7,8-tetrahydroimidazo[1,2-a]pyrazine-2-carboxylate (Step B) and 242 mg (0.768 mmol) of (3R)-3-[(tert-butoxycarbonyl)amino]-4-(2,5-difluorophenyl)butanoic acid (Intermediate 1) in 65 mL of DMF was added 125 mg (0.922 mmol) of HOBT and 177 mg (0.922 mmol) of EDC. The resultant mixture was stirred at ambient temperature for 14 h, and then partitioned between ethyl acetate and saturated aqueous sodium bicarbonate solution. The aqueous phase was extracted with three portions of ethyl acetate. The combined organic phases were washed with brine, dried over magnesium sulfate, and concentrated. Purification by flash chromatography on a Biotage® system (silica gel, eluting with 50% ethyl acetate/hexane, ethyl acetate, and 10% methanol/ethyl acetate) gave the title compound.

### Step D: Ethyl 7-[(3R)-3-amino-4-(2,5-difluorophenyl)butanoyl]-5,6,7,8-tetrahydroimidazo[1,2-α]pyrazine-2-carboxylate, trifluoroacetic acid salt

To a solution of 28 mg of ethyl 7-[(3R)-3-[(tert-butoxycarbonyl)amino]-4-(2,5-difluorophenyl)butanoyl]-5,6,7,8-tetrahydroimidazo[1,2-a]pyrazine-2-carboxylate in 0.5 mL of dichloromethane was added 0.5 mL of trifluoroacetic acid. The reaction mixture was stirred at ambient temperature for 1.5 h. Concentration gave the title compound as a solid. LC/MS 393 (M+1).

### EXAMPLE 2

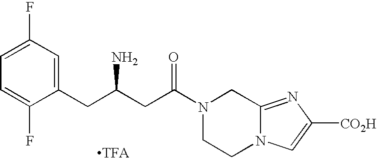

### 7-[(3R)-3-Amino-4-(2,5-difluorophenyl)butanoyl]-5,6,7,8-tetrahydroimidazo[1,2-a]pyrazine-2-carboxylic acid, trifluoroacetic acid salt

To a solution of 165 mg (0.335 mmol) of ethyl 7-[(3R)-3-[(tert-butoxycarbonyl)amino]4-(2,5-difluorophenyl)butanoyl2-5,6,7,8-tetrahydroimidazo[1,2-a]pyrazine-2-carboxylate (Example 1, Step C) in 2 mL of tetrahydrofuran and 2 mL of water was added lithium hydroxide (24 mg, 1.01 mmol). The reaction mixture was stirred at ambient temperature for 14 h. It was then concentrated and partitioned between ethyl acetate and 2N aqueous hydrochloric acid. The aqueous phase was washed sequentially with three portions of ethyl acetate. Concentration of the aqueous phase provided the title compound, which was purified by HPLC (YMC Pro-C18 column, gradient elution, 5-95% acetonitrile/water with 0.1% TFA) to give the title compound. LCIMS 365 (M+1).

### EXAMPLE 3

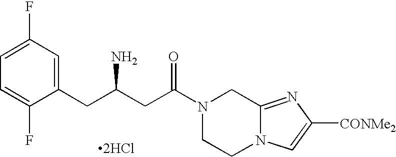

### 7-[(3R)-3-Amino-4-(2,5-difluorophenyl)butanoyl]-N,N-dimethyl-5,6,7,8-tetrahydroimidazo[1,2-a]pyrazine-2-carboxamide, dihydrochloride

### Step A: 7-[(3R)-3-[(tert-Butoxycarbonyl)amino]-4-(2,5-difluorophenyl)butanoyl]-5,6,7,8-tetrahydroimidazo[1,2-a]pyrazine-2-carboxylic acid

To a solution of 295 mg (0.6 mmol) of ethyl 7-[(3R)-3-[(tert-butoxycarbonyl)amino]-4-(2,5-difluorophenyl)butanoyl]-5,6,7,8-tetrahydroimidazo[1,2-a]pyrazine-2-carboxylate (Example 1, Step C) in 4 mL of tetrahydrofuran and 4 mL of water was added 57.7 mg (2.4 mmol) of lithium hydroxide. The mixture was stirred at ambient temperature for 14 h. It was then concentrated to a volume of approximately 4 mL. Acetic acid (0.173 mL) was added and the mixture was extracted sequentially with three portions of ethyl acetate. The combined organic phase was washed with brine, dried over magnesium sulfate, and concentrated to provide the title compound as a white solid.

### Step B: N,N-Dimethyl-7-[(3R)-3-[(tert-butoxycarbonyl)amino]-4-(2,5-difluorophenyl)butanoyl]-5,6,7,8-tetrahydroimidazo[1,2-α]pyrazine-2-carboxamide

To a solution of 50 mg (0.108 mmol) of 7-[(3R)-3-[(tert-butoxycarbonyl)amino]-4-(2,5-difluorophenyl)butanoyl]-5,6,7,8-tetrahydroimidazo[1,2-a]pyrazine-2-carboxylic acid (Step A) and 0.108 mL of a 2M solution of dimethylamine in tetrahydrofuran in 1 mL of DMF was added 17.5 mg (0.13 mmol) of HOBT and 24.9 mg (0.13 mmol) of EDC. The reaction mixture was stirred at ambient temperature for 18 h and then concentrated. Purification by chromatography (Biotage system, silica gel, eluting sequentially with ethyl acetate, 10% methanol/ethyl acetate, and 10% methanol/dichloromethane) provided the title compound as a white solid.

### Step C: 7-[(3R)-3-Amino-4-(2,5-difluorophenyl)butanoyl]-N,N-dimethyl-5,6,7,8-tetrahydroimidazo[1,2-a]pyrazine-2-carboxamide, dihydrochloride

A solution of 22 mg of N,N-dimethyl-7-[(3R)-3-[(tert-butoxycarbonyl)amino]-4-(2,5-difluorophenyl)butanoyl]-5,6,7,8-tetrahydroimidazo[1,2-a]pyrazine-2-carboxamide (Step B) in saturated methanolic hydrogen chloride was stirred at ambient temperature for 1 h. Concentration gave the title compound as a white solid. LC/MS 392 (M+1).

### EXAMPLE 4

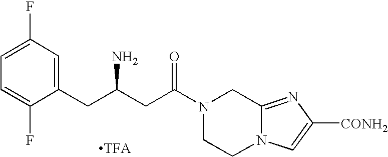

### 7-[(3R)-3-Amino-4-(2,5-difluorophenyl)butanoyl]-5,6,7,8-tetrahydroimidazo[1,2-a]pyrazine-2-carboxamide, trifluoroacetic acid salt

### Step A: 7-[(3R)-3-[(tert-Butoxcarbonyl)amino]-4(2,5-difluorophenyl)butanoyl]-5,6,7,8-tetrahydroimidazo[1,2-a]pyrazine-2-carboxamide

A solution of 100 mg of ethyl 7-[(3R)-3-[(tert-butoxycarbonyl)amino]-4-(2,5-difluorophenyl)butanoyl]-5,6,7,8-tetrahydroimidazo[1,2-a]pyrazine-2-carboxylate (Example 1, Step C) in 3.5 mL of concentrated ammonium hydroxide was heated in a sealed tube at 100° C. for 6 h. The reaction mixture was concentrated in vacuo and purified by chromatography (Biotage system, silica gel, 10% methanol/ethyl acetate) to give the title compound.

### Step B: 7-[(3R)-3-Amino-4-(2,5-difluorophenyl)butanoyl]-5,6,7,8-tetrahydroimidazo[1,2-a]pyrazine-2-carboxamide, trifluoroacetic acid salt

To a solution of 26 mg of 7-[(3R)-3-[(tert-butoxycarbonyl)amino]-4-(2,5-difluorophenyl)butanoyl]-5,6,7,8-tetrahydroimidazo[1,2-a]pyrazine-2-carboxamide (Step A) in 0.4 mL of dichloromethane was added 0.4 mL of trifluoroacetic acid. The reaction mixture was stirred at ambient temperature for 1 h and then concentrated to provide the title compound as a foamy solid.

### EXAMPLE 5

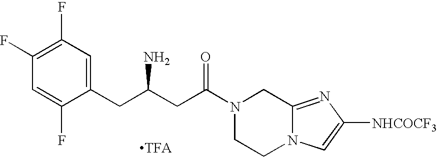

### 7-[(3R)-3-Amino-4-(2,4,5-trifluorophenyl)butanoyl]-2-(trifluoroacetylamino)-5,6,7,8-tetrahydroimidazo[1,2-a]pyrazine, trifluoroacetic acid salt

### Step A: 4-Methyl-N-[(2E)-pyrazine-2(1H)-ylidene]benzenesulfonamide

To a solution of 2-aminopyrazine (5.00 g, 52.5 mmol) in pyridine (40 mL) was added p-toluenesulfonyl chloride (11.0 g, 57.8 mmol). The reaction was stirred at ambient temperature for 80 min. After the pyridine was evaporated under vacuum, 0.5 L of water was added and the mixture was stirred at ambient temperature for 1 h. The reaction was filtered and the solid was washed sequentially with one portion of water and two portions of diethyl ether. The solid was dried under vacuum to give the title compound which was directly used in the next step without further purification. LC/MS 250 (M+1).

### Step B: 2-[(2E)-2-[[(4-Methylphenyl)sulfonyl]imino]pyrazin-1(2H)-yl]acetamide

To a suspension of 4.50 g (18.1 mmol) of 4-methyl-N-[(2E)-pyrazine-2(1H)-ylidene]benzenesulfonamide (Step A) in 30 mL of N,N-dimethylformamide was added 3.46 mL (19.9 mmol) of diisopropylethylamine and 3.67 g (19.9 mmol) of iodoacetamide. After the reaction was stirred at ambient temperature for 28 h, the mixture was poured into 20 mL of water and stirred for 100 min. The reaction mixture was filtered and the solid was washed sequentially with 200 mL of water and 100 mL of diethyl ether to afford the title compound as a solid.

### Step C: 2,2,2-Trifluoro-N-imidazo[1,2-a]pyrazin-2-ylacetamide

To a solution of 1.0 g (3.26 mmol) of 2-[(2E)-2-[[(4-methylphenyl)sulfonyl]imino]pyrazin-1(2H)-yl]acetamide (Step B) in 15 mL of dichloromethane was added 6 mL of trifluoroacetic anhydride. After the reaction was refluxed for 6 h, the solvent was evaporated and 30 mL of ethyl acetate was added. The mixture was filtered and the solid was washed with ethyl acetate to give the title compound as a solid. ¹H NMR (500 MHz, DMSO-d₆): δ 9.13 (s, 1H), 8.62 (d, 1H), 8.38 (s, 1H), 8.08 (d, 1H.).

### Step D: 2,2,2-Trifluoro-N-(5,6,7,8-tetrahydroimidazo[1,2-a]pyrazin-2-yl)acetamide

The title compound was prepared from 2,2,2-trifluoro-N-imidazo[1,2-a]pyrazin-2-ylacetamide (Step C) using a procedure analogous to that of Example 1, Step B.

### Step E: 7-[(3R)-3-[(tert-Butoxycarbonyl)amino]-4-(2,4,5-trifluorophenyl)butanoyl-2-(trifluoroacetylamino)-5,6,7,8-tetrahydroimidazo[1,2-a]pyrazine

The title compound was prepared from 2,2,2-trifluoro-N-(5,6,7,8-tetrahydroimidazo[1,2-a]pyrazin-2-yl)acetamide (Step D) and (3R)-3-[(tert-butoxycarbonyl)amino]-4-(2,4,5-trifluorophenyl)butanoic acid (Intermediate 3) using a procedure analogous to that of Example 1, Step C. LC/MS 550 (M+1).

### Step F: 7-[(3R)-3-Amino-4-(2,4,5-trifluorophenyl)butanoyl]-2-(trifluoroacetylamino)-5,6,7,8-tetrahydroimidazo[1,2-a]pyrazine, trifluoroacetic acid salt

The title compound was prepared from 7-[(3R)-3-[(tert-butoxycarbonyl)amino]-4-(2,4,5-trifluorophenyl)butanoyl]-2-(trifluoroacetylamino)-5,6,7,8-tetrahydroimidazo[1,2-a]pyrazine (from Step E) using a procedure analogous to that of Example 1, Step D. LC/MS 450 (M+1).

### EXAMPLE 6

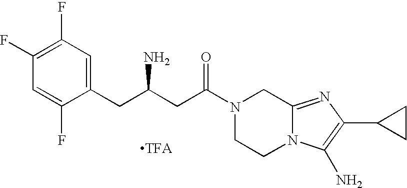

### 3-Amino-7-[(3R)-3-amino-4-(2,4,5-difluorophenyl)butanoyl]-2-cyclopropyl-5,6,7,8-tetrahydroimidazo]1,2-a]pyrazine, trifluoroacetic acid salt

### Step A: 2-Cyclopropyl-N-(1-isopropyl-3,3-dimethylbutyl)imidazo[1,2-a]pyrazin-3-amine

To a solution of 2-aminopyrazine (1.13 g, 11.9 mmol) in methanol (12 mL) and dichloromethane (12 mL) was added 1,1,3,3-tetramethylbutyl isocyanide (2.50 mL, 14.3 mmol), cyclopropanecarboxaldehyde (1.00 g, 14.3 mmol) and scandium triflate (292 mg, 0.595 mmol). The reaction was stirred at ambient temperature for 24 h. The reaction mixture was concentrated under vacuum and purified by flash chromatography (silica gel, 100% dichloromethane then 10% methanol in dichloromethane) to afford the title compound as a solid. LC/MS 287 (M+1).

### Step B: 2-Cyclopropyl-N-(1,1,3,3-tetramethylbutyl)-5,6,7,8-tetrahydroimidazo[1,2-a]pyrazin-3-amine

The title compound was prepared from 2-cyclopropyl-N-(1-isopropyl-3,3-dimethylbutyl)imidazo[1,2-a]pyrazin-3-amine (Step A) using a procedure analogous to that of Example 1, Step B. LC/MS 291 (M+1).

### Step C: 7-[(3R)-3-[(tert-Butoxycarbonyl)amino]-4-(2,4,5-trifluorophenyl)butanoyl]-2-cyclopropyl-3-[(1,1,3,3-tetramethylbutyl)amino]-5,6,7,8-tetrahydroimidazo[1,2-a]pyrazine

The title compound was prepared from 2-cyclopropyl-N-(1,1,3,3-tetramethylbutyl)-5,6,7,8-tetrahydroimidazo[1,2-a]pyrazin-3-amine (Step B) and (3R)-3-[(tert-butoxycarbonyl)amino]-4-(2,4,5-trifluorophenyl)butanoic acid (Intermediate 3) using a procedure analogous to that of Example 1, Step C. LC/MS 606 (M+1).

### Step D: 3-Amino-7-[(3R)-3-amino-4-(2,4,5-trifluorophenyl)butanoyl]-2-cyclopropyl-5,6,7,8-tetrahydroimidazo[1,2-a]pyrazine, trifluoroacetic acid salt

To a solution of 35.0 mg of 7-[(3R)-3-[(tert-butoxycarbonyl)amino]-4-(2,4,5-trifluorophenyl)butanoyl]-2-cyclopropyl-3-[(1,1,3,3-tetramethylbutyl)amino]-5,6,7,8-tetrahydroimidazo[1,2-a]pyrazine (Step C) in 1 mL of dichloromethane was added 1 mL of trifluoroacetic acid. The reaction was stirred at ambient temperature for 1 h. The mixture was concentrated and purified by reverse-phase HPLC (YMC Pro-C18 column, gradient elution, 10-30% acetonitrile/water with 0.1% TFA) to give the title compound as a solid. LC/MS 394 (M+1).

### EXAMPLE 7

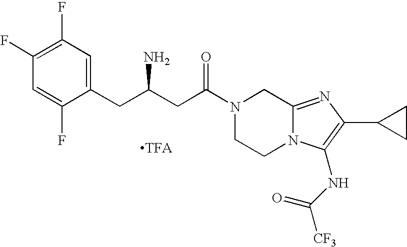

### 7-[(3R)-3-Amino-4-(2,4,5-trifluorophenyl)butanoyl]-2-cyclopropyl-3-(2,2,2-trifluoroacetylamino)-5,6,7,8-tetrahydroimidazo[1,2-a]pyrazine, trifluoroacetic acid salt

### Step A: 2-Cyclopropylimidazo[1,2-a]pyrazin-3-amine

To a solution of 1.50 g (8.06 mmol) of 2-cyclopropyl-N-(1-isopropyl-3,3-dimethylbutyl)imidazo[1,2-a]pyrazin-3-amine (Example 6, Step A) in 4 mL of dichloromethane was added 4 mL of trifluoroacetic acid and the reaction was stirred at ambient temperature for 1 h. The mixture was concentrated in vacuo to give the title compound as a viscous oil. LC/MS 175 (M+1).

### Step B: N-(2-Cyclopropylimidazo[1,2-a]pyrazin-3-yl)-2,2,2-trifluoroacetamide

To a solution of 350 mg (1.22 mmol) of 2-cyclopropylimidazo[1,2-a]pyrazin-3-amine (Step A) in 5 mL of dichloromethane at 0° C. was added pyridine (0.628 mL, 7.29 mmol) and trifluoroacetic anhydride (0.513 mL, 3.65 mmol). The reaction was stirred at 0° C. for 1 h. After the mixture was concentrated at 0° C. under vacuum, 1 mL of ice-cold water was added. The reaction mixture was then purified by reverse-phase HPLC (YMC Pro-C18 column, gradient elution, 10-90% acetonitrile/water with 0.1% TFA) to afford the title compound as a solid. LC/MS 271 (M+1).

### Step C: N-(2-Cyclopropyl-5,6,7,8-tetrahydroimidazo]1,2-a]pyrazin-3-yl)-2,2,2-trifluoroacetamide

The title compound was prepared from N-(2-cyclopropylimidazo[1,2-a]pyrazin-3-yl)-2,2,2-trifluoroacetamide (Step B) using a procedure analogous to that of Example 1, Step B.

### Step D: 7-[(3R)-3-[(tert-Butoxycarbonyl)amino]-4-(2,4,5-trifluorophenyl)butanoyl]-2-cyclopropyl-3-(2,2,2-trifluoroacetylamino)-5,6,7,8-tetrahydroimidazo]1,2-a]pyrazine

The title compound was prepared from N-(2-cyclopropyl-5,6,7,8-tetrahydroimidazo[1,2-a]pyrazin-3-yl)-2,2,2-trifluoroacetamide (Step C) and (3R)-3-[(tert-butoxycarbonyl)amino]-4-(2,4,5-trifluorophenyl)butanoic acid (Intermediate 3) using a procedure analogous to that of Example 1, Step C. LC/MS 590 (M+1).

### Step E: 7-[(3R)-3-Amino-4-(2,4,5-trifluorophenyl)butanoyl]-2-cyclopropyl-3-(2,2,2-trifluoroacetylamino)-5,6,7,8-tetrahydroimidazo[1,2-a]pyrazine, trifluoroacetic acid salt

The title compound was prepared from 7-[(3R)-3-[(tert-butoxylcarbonyl)amino]-4-(2,4,5-trifluorophenyl)butanoyl]-2-cyclopropyl-3-(2,2,2-trifluoroacetylamino)-5,6,7,8-tetrahydroimidazo[1,2-a]pyrazine(from Step D) using a procedure analogous to that of Example 1, Step D. LC/MS 490 (M+1).

### EXAMPLE 8

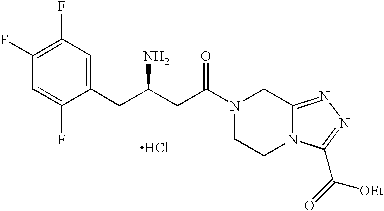

### Ethyl 7-[(3R)-3-amino-4-(2,4,5-trifluorophenyl)butanoyl-5,6,7,8-tetrahydro1,2,4]triazolo[4,3-a]pyrazine-3-carboxylate, hydrochloride

### Step A: Ethyl oxo(2-pyrazin-2-ylhydrazino)acetate

To a mixture of 2.38 g (21.6 mmol) of 2-hydrazinopyrazine and 2.41 g (3.32 mL, 23.8 mmol) of triethylamine in 50 mL of acetonitrile was added 3.25 g (2.66 mL, 23.8 mmol) of ethyl chloro(oxo)acetate at 0° C. The reaction mixture was stirred at ambient temperature for 18 h, and then partitioned between aqueous sodium bicarbonate solution and ethyl acetate. The aqueous phase was extracted with three portions of ethyl acetate. The combined organic phases were washed with brine, dried over magnesium sulfate, and concentrated to give the title compound. LC/MS 211 (M+1).

### Step B: Ethyl [1,2,4]triazolo[4,3-a]pyrazine-3-carboxylate

To a solution of 521 mg (2.48 mmol) of ethyl oxo(2-pyrazin-2-ylhydrazino)acetate from Step A in 10 mL of toluene was added 20 mg of p-toluenesulfonic acid. The mixture was heated at reflux for 18 h. The reaction mixture was washed with aqueous sodium bicarbonate solution. The aqueous phase was extracted with three portions of ethyl acetate. The combined organic phases were washed with brine, dried over magnesium sulfate, and concentrated to give the title compound. LC/MS 192.9 (M+1).

### Step C: Ethel 5,6,7,8-tetrahydro[1,2,4]triazolo[4,3-a]pyrazine-3-carboxylate

A mixture of 380 mg of ethyl [1,2,4]triazolo[4,3-a]pyrazine-3-carboxylate from Step B and 50 mg of 10% palladium on carbon in 10 mL of ethyl acetate and 10 mL of ethanol was stirred under an atmosphere of hydrogen for 18 h. The mixture was filtered through a pad of Celite and the filtrate concentrated to give the title compound as a solid. LC/MS 169 (M+1).

### Step D: Ethyl 7-[(3R)-3-[(tert-butoxycarbonyl)amino]-4-(2,4,5-trifluorophenyl)butanoyl]-5,6,7,8-tetrahydro[1,2,4]triazolo[4,3-a]pyrazine-3-carboxylate

A 264 mg (1.35 mmol) portion of ethyl 5,6,7,8-tetrahydro[1,2,4]triazolo[4,3-a]pyrazine-3-carboxylate from Step C was coupled to (3R)-3-[(tert-butoxycarbonyl)amino]-4-(2,4,5-trifluorophenyl)butanoic acid (Intermediate 3) essentially following the procedure outlined in Example 1, Step C. Purification by flash chromatography on a Biotage® system (silica gel, eluting with 5% methanol/dichloromethane) gave the title compound. LC/MS 456 (M+1-tBu).

### Step E: Ethyl 7-[(3R)-3-amino-4-(2,4,5-trifluorophenyl)butanoyl]-5,6,7,8-tetrahydro[1,2,4]triazolo[4,3-a]pyrazine-3-carboxylate, hydrochloride

A 10 mg portion of ethyl 7-[(3R)-3-[(tert-butoxycarbonyl)amino]-4-(2,4,5-trifluorophenyl)butanoyl]-5,6,7,8-tetrahydro[1,2,4]triazolo[4,3-a]pyrazine-3-carboxylate from Step D was deprotected essentially following the procedure outlined in Example 3, Step C to provide the title compound. LC/MS 412 (M+1).

### EXAMPLE 9

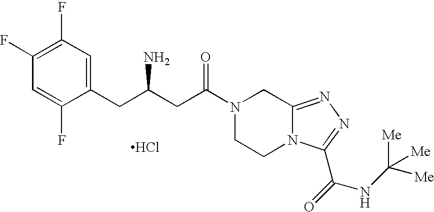

### N-(tert-Butyl)-7-[(3R)-3-amino-4-(2,4,5-trifluorophenyl)butanoyl]-5,6,7,8-tetrahydro1,2,4]triazolo[4,3-a]pyrazine-3-carboxamide, hydrochloride

### Step A: N-(tert-Butyl)[1,2,4]triazolo[4,3-a]pyrazine-3-carboxamide

A mixture of 170 mg of ethyl [1,2,4]triazolo[4,3-a]pyrazine-3-carboxylate from Example 8, Step B and 3 mL of tert-butylamine was heated in a sealed tube at reflux for 8 h. Concentration followed by flash chromatography (silica gel, eluting sequentially with 100% ethyl acetate and 10% methanol/dichloromethane) gave the title compound as a viscous oil. LC/MS 219.9 (M+1).

### Step B: N-(tert-Butyl)-5,6,7,8-tetrahydro[1,2,4]triazolo[4,3-a]pyrazine-3-carboxamide

A mixture from 167 mg of N-(tert-butyl)[1,2,4]triazolo[4,3-a]pyrazine-3-carboxamide from Step A and 20 mg of 10% palladium on carbon in 3 mL of ethanol was stirred under an atmosphere of hydrogen for 18 h. The mixture was filtered through a pad of Celite and the filtrate concentrated to give the title compound as a solid. ¹H NMR (CDCl₃): δ 7.22 (1H, s), 4.43 (2H, t, J=5.5 Hz), 4.32 (2H, s), 3.27 (2H, t, J=5.5 Hz), 2.05 (1H, br), 1.48 (9H, s).

### Step C: N-(tert-Butyl)-7-[(3R)-3-[(tert-butoxycarbonyl)amino]-4-(2,4,5-trifluorophenyl)butanoyl]-5,6,7,8-tetrahydro]1,2,4]triazolo[4,3-a]pyrazine-3-carboxamide

A 32 mg (0.14 mmol) portion of N-(tert-butyl)-5,6,7,8-tetrahydro[1,2,4]triazolo[4,3-a]pyrazine-3-carboxamide from Step B was coupled to (3R)-3-[(tert-butoxycarbonyl)amino]-4-(2,4,5-trifluorophenyl)butanoic acid (Intermediate 3) essentially following the procedure outlined in Example 1, Step C. Purification by preparative TLC (silica gel, 10% methanol/dichloromethane) gave the title compound. LC/MS 483.1 (M+1-tBu).

Step D: N-(tert-Butyl)-7-[(3R)-3-amino-4-(2,4,5-trifluorophenyl)butanol]-5,6,7,8-tetrahydro[1,2,4]triazolo[4,3-a]pyrazine-3-carboxamide, hydrochloride

A 63 mg portion of N-(tert-butyl)-7-[(3R)-3-[(tert-butoxycarbonyl)amino]-4-(2,4,5-trifluorophenyl)butanoyl]-5,6,7,8-tetrahydro[1,2,4]triazolo[4,3-a]pyrazine-3-carboxamide from Step C was deprotected essentially following the procedure outlined in Example 3, Step C to provide the title compound. LC/MS 439 (M+1).

### EXAMPLE 10

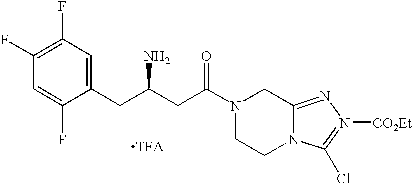

### Ethyl 7-[(3R)-3-amino-4-(2,4,5-trifluorophenyl)butanoyl]-3-chloro-5,6,7,8-tetrahydroimidazo[1,2-a]pyrazine-2-carboxylate, trifluoroacetic acid salt

### Step A: Ethyl 7-(tert-butoxycarbonyl)-5,6,7,8-tetrahydroimidazo[1,2-a]pyrazine-2-carboxylate

To ethyl 5,6,7,8-tetrahydroimidazo[1,2-a]pyrazine-2-carboxylate (Example 1, Step B, 23.8 g, 122 mmol) in 400 mL of dichloromethane was added di-tert-butyl dicarbonate (29.3 g, 134 mmol). The reaction mixture was stirred at ambient temperature for 2 h then concentrated in vacuo. Purification by flash chromatography (silica gel, gradient elution, 70% ethyl acetate/hexane to 100% ethyl acetate to 10% methanol/ethyl acetate) afforded the title compound.

### Step B: Ethel 7-(tert-butoxycarbonyl)-3-chloro-5,6,7,8-tetrahydroimidazo[1,2-a]pyrazine-2-carboxylate

To a solution of ethyl 7-(tert-butoxycarbonyl)-5,6,7,8-tetrahydroimidazo[1,2-a]pyrazine-2-carboxylate (4.28 g, 14.5 mmol) from Step A in 100 mL of carbon tetrachloride was added N-chlorosuccinimide (2.325 g, 17.4 mmol) and benzoyl peroxide (50 mg, 0.2 mmol) sequentially. The reaction was stirred at reflux for 1 h. The reaction mixture was cooled to 0° C., filtered, and the solid was washed with two 25-mL portions of dichloromethane. The solvent was concentrated in vacuo. Purification by flash chromatography on a Biotage® system (silica gel, gradient, 50% ethyl acetate/hexane to 100% ethyl acetate) afforded the title compound.

### Step C: Ethyl 3-chloro-5,6,7,8-tetrahydroimidazo]1,2-a]pyrazine-2-carboxylate, trifluoroacetic acid salt

To a solution of 180 mg of ethyl 7-(tert-butoxycarbonyl)-3-chloro-5,6,7,8-tetrahydroimidazo[1,2-a]pyrazine-2-carboxylate from Step B in 5 mL of dichloromethane was added 5 mL of trifluoroacetic acid. The reaction mixture was stirred at ambient temperature for 1.5 h. Concentration gave the title compound.

### Step D: Ethyl 7-[(3R)-3-[(tert-butoxycarbonyl)amino]-4-(2,4,5-trifluorophenyl)butanoyl]-3-chloro-5,6,7,8-tetrahydroimidazo[1,2-a]pyrazine-2-carboxylate

To a solution of ethyl 3-chloro-5,6,7,8-tetrahydroimidazo[1,2-a]pyrazine-2-carboxylate, trifluoroacetic acid salt (185 mg, 0.54 mmol) with (3R)-3-[(tert-butoxycarbonyl)amino]-4-(2,4,5-trifluorophenyl)butanoic acid (95 mg, 0.30 mmol) and N,N-diisopropylethylamine (DIEA) (0.188 mL, 1.1 mmol) in 8 mL of dichloromethane was added HOAT (41 mg, 0.30 mmol) and HATU (113 mg, 0.30 mmol). The reaction mixture was stirred at ambient temperature for 12 h. The reaction mixture was filtered though a plug of PSA resin (200 mg; Varian MEGA BOND ELUT, PSA) and the resin washed with 10 mL of a 10% methanol/dichloromethane solution. The filtrate was concentrated in vacuo. Purification by reverse phase HPLC (YMC Pro-C18 column, gradient elution, 10-90% acetonitrile/water with 0.1% TFA) afforded title compound as a white foam.

### Step E: Ethyl 7-[(3R)-3-amino-4-(2,4,5-trifluorophenyl)butanoyl]-3-chloro-5,6,7,8-tetrahydroimidazo[1,2-a]pyrazine-2-carboxylate, trifluoroacetic acid salt

To a solution of ethyl 7-[(3R)-3-[(tert-butoxylcarbonyl)amino]4-(2,4,5-trifluorophenyl)butanoyl]-3-chloro-5,6,7,8-tetrahydroimidazo[1,2-a]pyrazine-2-carboxylate (67 mg, 0.012 mmol) in 1 mL of dichloromethane was added 1 mL of trifluoroacetic acid. The reaction mixture was stirred at ambient temperature for 0.5 h and concentrated in vacuo. Purification by HPLC (YMC Pro-C18 column, gradient elution, 10-90% acetonitrile/water with 0.1% TFA) provided the title compound as a white foam. MS 445.2 (M+1).

### EXAMPLE 11

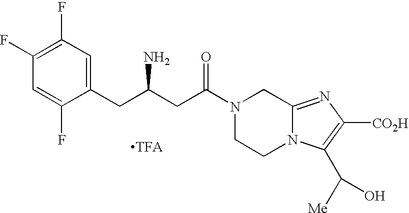

### 7-[(3R)-3-Amino-4-(2,4,5-trifluorophenyl)butanoyl]-3-[(R or S)-1-hydroxyethyl]-5,6,7,8-tetrahydroimidazo[1,2-a]pyrazine-2-carboxylic acid, trifluoroacetic acid salt and 7-[(3R)-3-Amino-4-(2,4,5-trifluorophenyl)butanoyl]-3-[(S or R)-1-hydroxyethyl]-5,6,7,8-tetrahydroimidazo[1,2-a]pyrazine-2-carboxylic acid, trifluoroacetic acid salt

### Step A: Ethyl 3-bromo-7-(tert-butoxycarbonyl)-5,6,7,8-tetrahydroimidazo[1,2-a]pyrazine-2-carboxylate

To a chloroform solution (250 mL) of ethyl 7-(tert-butoxycarbonyl)-5,6,7,8-tetrahydroimidazo[1,2-a]pyrazine-2-carboxylate from Example 10, Step A (18.4 g, 94 mmol), bromine (45 g, 281 mmol) was added, and the mixture was stirred at room temperature for an hour. An aqueous solution of sodium bisulfite was then added and the product was extracted with two 100-mL portions of dichloromethane. The combined organic extracts were washed sequentially with sodium bicarbonate and brine, then dried over sodium sulfate and concentrated in vacuo. The crude material was purified by flash chromatography (silica gel, gradient elution, 50% ethyl acetate/hexane to 100% ethyl acetate) to give the title compound.

### Step B: Ethyl 7-(tert-butoxycarbonyl)-3-(1-ethoxyvinyl)-5,6,7,8-tetrahydroimidazo[1,2-a]pyrazine-2-carboxylate

The bromide from Step A (8.0 g, 21 mmol), tributyl(1-ethoxyvinyl)tin (8.7 mL, 26 mmol), tris(dibenzylideneacetone)dipalladium (0.98 g, 1.1 mmol) and tri-2-furylphosphine (0.99 g, 4.3 mmol) were stirred in dioxane (200 ml) under nitrogen at 110° C. for 6 h. After cooling to room temperature, 200 mL of an aqueous solution of potassium fluoride (3.0 g) was added and the resulting mixture was extracted with three 200-mL portions of ethyl acetate. The combined organic extracts were washed sequentially with water and brine, then dried over sodium sulfate, filtered, and concentrated. The crude material was purified by flash chromatography (silica gel, 50% ethyl acetate/hexane to 100% ethyl acetate) to obtain the title compound.

### Step C: Ethyl 3-acetyl-7-(tert-butoxycarbonyl)-5,6,7,8-tetrahydroimidazo[1,2-a]pyrazine-2-carboxylate

To the enol ether from Step B (5.78 g, 15.8 mmol) in 200 mL of a 10:1 mixture of THF/water, para-toluenesulfonic acid (300 mg) was added. The reaction mixture was heated at 50° C. for 2 hours. The reaction mixture was then cooled to room temperature, concentrated in vacuo, and treated with 400 mL of ethyl acetate. The organic solution was washed sequentially with two 400-mL portions of aqueous saturated sodium bicarbonate and one 400-mL portion of brine, dried over sodium sulfate, filtered, and concentrated in vacuo to give the title compound which was used without further purification.

### Step D: Ethyl 7-(tert-butoxycarbonyl)-3-(1-hydroxyethyl)-5,6,7,8-tetrahydroimidazo[1,2-a]pyrazine-2-carboxylate

The ketone from Step C (5.6 g, 17 mmol) was dissolved in 200 mL of methanol and the solution was cooled to 0° C. Sodium borohydride (6.3 g, 166 mmol) was added portionwise and the reaction mixture was allowed to stir at room temperature for 1 h. The reaction mixture was then concentrated in vacuo and treated with 400 mL of ethyl acetate, washed once with brine (400 mL), dried over sodium sulfate, filtered, and concentrated. Purification by flash chromatography on a Biotage® system (silica gel, gradient elution, 70% ethyl acetate/hexane to 100% ethyl acetate) afforded the title compound as a racemic mixture. The racemate was resolved by chiral HPLC (ChiralCel OD (4.6×250 mm) 10 micron column, 15% ethanol/hexane) to give the two enantiomers.

### Step E: 7-(tert-Butoxycarbonyl)-3-[(R or S)-1-hydroxyethyl]-5,6,7,8-tetrahydroimidazo[1,2-a]pyrazine-2-carboxylic acid

The faster eluting enantiomer (Enantiomer A) from Step D (223 mg, 0.65 mmol) in 15 mL of a 3:1:1 mixture of tetrahydrofuran:methanol:1N aqueous lithium hydroxide solution was stirred at ambient temperature for 4 h. The THF was evaporated under reduced pressure. The reaction mixture acidified with 5% hydrochloric acid, then extracted with ethyl acetate (3×30 mL). The combined organic phases were washed with brine, dried over magnesium sulfate and concentrated in vacuo. Purification by HPLC (YMC Pro-C18 column, gradient elution, 10-90% acetonitrile/water with 0.1% TFA) afforded the title compound.

The same procedure was used to obtain the acid of the slower eluting enantiomer (Enantiomer B) from Step D.

### Step F: 6-(tert-Butoxycarbonyl)-1-methyl-5,6,7,8-tetrahydrofuro[3′,4′:4,5imidazo[1,2-a]pyrazin-3(1H)-one, trifluoroacetic acid salt

Enantiomer A from Step E (173 mg, 0.56 mmol) in 2 mL of acetonitrile with triethylamine (0.6 mL, 4 mmol) was added dropwise over 5 min to N-methyl chloropyridinium iodide (568 mg, 2.22 mmol) in 8 mL of refluxing acetonitrile. The reaction mixture was then cooled to room temperature, 50 mL of dichloromethane was added, and the organic solution was washed sequentially with 50 mL of water and 50 mL of brine, dried over sodium sulfate and concentrated in vacuo. Purification by HPLC (YMC Pro-C18 column, gradient elution, 10-90% acetonitrile/water with 0.1% TFA) afforded the title compound.

The same procedure was used to obtain the lactone of Enantiomer B.

### Step G: 7-[(3R)-3-[(tert-Butoxycarbonyl)amino]-4-(2,4,5-trifluorophenyl)butanoyl-3-[(R or S)-1-hydroxyethyl]-5,6,7,8-tetrahydroimidazo[1,2-a]pyrazine-2-carboxylic acid

Enantiomer A from Step F was treated with 8 mL of 4N hydrogen chloride solution in dioxane. The resultant mixture was stirred at ambient temperarture for 2 h then concentrated in vacuo to give the crude HCl salt.

To a solution of HCl salt of isomer A (72.5 mg, 0.25 mmol), (3R)-3-[(tert-butoxycarbonyl)amino]-4-(2,4,5-trifluorophenyl)butanoic acid (85 mg, 0.25 mmol, Intermediate 3), and N,N-diisopropylethylamine (0.09 mL, 0.50 mmol) in 3 mL of dichloromethane was added HOAT (34 mg, 0.25 mmol) and HATU (95 mg, 0.25 mmol). The resulting mixture was stirred at ambient temperature for 12 h. The reaction mixture was filtered though a plug of PSA resin (200 mg of resin; Varian MEGA BOND ELUT PSA) and the resin washed with 7 mL of a 10% methanol/dichloromethane solution. The filtrate was concentrated in vacuo. Purification by HPLC (YMC Pro-C18 column, gradient elution, 10-90% acetonitrile/water with 0.1% TFA) gave the title compound.

The same procedure was used to obtain 7-[(3R)-3-[(tert-butoxycarbonyl)amino]-4-(2,4,5-trifluorophenyl)butanoyl]-3-[(S or R)-1-hydroxyethyl]-5,6,7,8-tetrahydroimidazo[1,2-a]pyrazine-2-carboxylic acid from Enantiomer B of Step F (Diastereoisomer B).

### Step H: 7-[(3R)-3-Amino-4-(2,4,5-trifluorophenyl)butanoyl]-3-[(R or S)-1-hydroxyethyl]-5,6,7,8-tetrahydroimidazo[1,2-a]pyrazine-2-carboxylic acid, trifluoroacetic acid salt

To a solution of 7-[(3R)-3-[(tert-butoxycarbonyl)amino]-4-(2,4,5-trifluorophenyl)butanoyl]-3-[(R or S)-1-hydroxyethyl]-5,6,7,8-tetrahydroimidazo[1,2-a]pyrazine-2-carboxylic acid (68 mg, 0.13 mmol) in 1 mL of dichloromethane was added 1 mL of trifluoroacetic acid. The reaction mixture was stirred at ambient temperature for 30 min, then concentrated in vacuo. Purification by HPLC (YMC Pro-C18 column, gradient elution, 10-90% acetonitrile/water with 0.1% TFA) afforded the title compound as a white foam. MS 427.1 (M+1).

The same procedure was used to obtain 7-[(3R)-3-amino-4-(2,4,5-trifluorophenyl)butanoyl]-3-[(S or R)-1-hydroxyethyl]-5,6,7,8-tetrahydroimidazo[1,2-a]pyrazine-2-carboxylic acid, trifluoroacetic acid salt from Diastereoisomer B of Step G.

### EXAMPLE 12

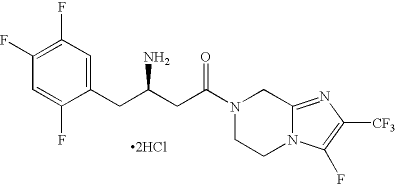

### 7-[(3R)-3-Amino-4-(2,4,5-trifluorophenyl)butanoyl]-3-fluoro-2-trifluoromethyl-5,6,7,8-tetrahydroimidazo[1,2-a]pyrazine, dihydrochloride

### Step A: 3-Fluoro-2-(trifluoromethyl)imidazo[1,2-a]pyrazine

To a solution of 1.00 g (10.5 mmol) of 2-aminopyrazine in 20 mL of toluene at −78° C. was added 200 mg of activated molecular sieves. The mixture was aerated with gaseous hexafluoroacetone for 3 min, then sealed and warmed to ambient temperature for 16 h. The reaction vessel was unsealed and aerated with nitrogen for 3 min. The resulting solution was filtered and concentrated in vacuo at ambient temperature. The residue was dissolved in 40 mL of xylenes, treated with a solution of 1.99 g (10.5 mmol) of tin(II) choride in 20 mL of tetrahydrofuran, and heated to 120° C. for 33 h. The mixture was cooled to ambient temperature and concentrated to minimum volume in vacuo. The resulting slurry was suspended in 30 mL of ethanol, and to this mixture was added a solution of 3.00 g (52.0 mmol) of potassium fluoride. After 30 min at ambient temperature, the mixture was diluted with diethyl ether and washed sequentially with saturated aqueous sodium bicarbonate and brine. The organic layer was dried over magnesium sulfate and concentrated in vacuo at ambient temperature to 5 mL total volume. This residue was purified by flash chromatography (silica gel, 40-65% gradient diethyl ether in hexanes) to afford the title compound. LC/MS 206 (M+1).

### Step B: 3-Fluoro-2-(trifluoromethyl)-5,6,7,8-tetrahydroimidazo[1,2-a]pyrazine

To a solution of 92.4 mg (0.451 mmol) of 3-fluoro-2-(trifluoromethyl)imidazo[1,2-a]pyrazine in 2 mL of DMF was added 142 mg (2.25 mmol) of sodium cyanoborohydride, and 0.20 mL of acetic acid. The mixture was heated to 70° C. for 1 h, then cooled to ambient temperature. The solution was diluted with 50 mL of 1N aqueous hydrochloric acid and extracted with ethyl acetate. The aqueous layer was then brought to pH 8.5 with saturated aqueous sodium bicarbonate solution and treated to saturation with solid sodium chloride. This mixture was extracted with ethyl acetate, and the organic layer was dried over magnesium sulfate and concentrated in vacuo, affording the title compound. LC/MS 210.0 (M+1).

### Step C: 7-[(3R)-3-[(tert-Butoxycarbonyl)amino]-4-(2,4,5-trifluorophenyl)butanoyl]-3-fluoro-2-trifluoromethyl-5,6,7,8-tetrahydroimidazo[1,2-a]pyrazine

To a solution of 54.7 mg (0.262 mmol) of 3-fluoro-2-(trifluoromethyl)-5,6,7,8-tetrahydroimidazo[1,2-a]pyrazine in 2.0 mL of DMF was added 95.9 mg (0.288 mmol) of (3R)-3-[(tert-butoxycarbonyl)amino]-4-(2,4,5-trifluorophenyl)butanoic acid (Intermediate 3), followed by 0.233 mL (1.31 mmol) of N,N-diisopropylethylamine, 39.2 mg (0.288 mmol) of 1-hydroxy-7-azabenzotriazole (HOAT), and 109 mg (0.288 mmol) of HATU reagent. After 1 h at ambient temperature, the reaction was diluted with 0.5M aqueous sodium bicarbonate solution and extracted with ethyl acetate. The organic layer was washed with brine, dried over magnesium sulfate, and concentrated in vacuo. Purification by flash chromatography (silica gel, 40-55% ethyl acetate in hexanes) afforded the title compound. LC/MS 547 (M+Na).

### Step D: 7-[(3R)-3-Amino-4-(2,4,5-trifluorophenyl)butanoyl]-3-fluoro-2-trifluoromethyl-5,6,7,8-tetrahydroimidazo[1,2-a]pyrazine, dihydrochloride

To a solution of 82.0 mg (0.156 mmol) of 7-[(3R)-3-[(tert-butoxycarbonyl)amino]-4-(2,4,5-trifluorophenyl)butanoyl]-3-fluoro-2-trifluoromethyl-5,6,7,8-tetrahydroimidazo[1,2-a]pyrazine in 1.8 mL of dichloromethane was added 0.2 mL of trifluoroacetic acid. After 2 h at ambient temperature the reaction was concentrated in vacuo. The residue was suspended in 10 mL of diethyl ether and treated with 0.5 mL of 1N hydrogen chloride in diethyl ether. The resulting suspension was concentrated in vacuo, affording the title compound. LC/MS 425 (M+1).

### EXAMPLE 13

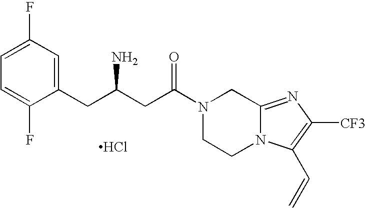

### 7-[(3R)-3-Amino-4-(2,5-difluorophenyl)butanoyl]-2-(trifluoromethyl)-3-vinyl-5,6,7,8-tetrahydroimidazo[1,2-a]pyrazine, hydrochloride

### Step A: 2-(Trifluoromethyl)-5,6,7,8-tetrahydroimidazo[1,2-a]pyrazine

The title compound was prepared from 2-aminopyrazine using a procedure analogous to that of Example 1, Steps A and B.

### Step B: 7-(tert-Butoxycarbonyl)-2-(trifluoromethyl)-5,6,7,8-tetrahydroimidazo[1,2-a]pyrazine

To a solution of 1.0 g (5.23 mmol) 2-(trifluoromethyl)-5,6,7,8-tetrahydroimidazo[1,2-a]pyrazine (Step A) in 50 mL of dichloromethane at 0° C. was added N,N-diisopropylethylamine (0.910 mL, 5.23 mmol) and di-tert-butyl dicarbonate (1.14 g, 5.24 mmol). After the reaction was stirred at 0° C. for 5 min, the reaction was warmed to ambient temperature and continued to stir for 4 h. The reaction mixture was partitioned between ethyl acetate and 0.5N hydrochloric acid. The aqueous phase was extracted with three potions of ethyl acetate. The combined organic layers were washed with brine, dried over magnesium sulfate, and concentrated. The residue was purified by flash chromatography using a Biotage® system (silica gel, 50% ethyl acetate in hexanes then 100% ethyl acetate) to give the title compound as a solid. LC/MS 236 (M+1-56).

### Step C: 3-Bromo-7-(tert-butoxycarbonyl)-2-(trifluoromethyl)-5,6,7,8-tetrahydroimidazo[1,2-a]pyrazine

To a solution of 1.65 g (5.67 mmol) of 7-(tert-butoxycarbonyl)-2-(trifluoromethyl)-5,6,7,8-tetrahydroimidazo[1,2-a]pyrazine (Step B) in 100 mL of chloroform at 0° C. was added neat bromine slowly, and the reaction was stirred at 0° C. for 0.5 h. After the reaction was warmed to ambient temperature and stirred for 1 h, the mixture was quenched with saturated aqueous sodium bicarbonate solution. The aqueous phase was extracted with three potions of ethyl acetate. The combined organics were washed with brine, dried over magnesium sulfate, and concentrated. The residue was purified by flash chromatography on a Biotage system® (silica gel, 10% ethyl acetate in hexanes then 20% ethyl acetate in hexanes) to provide the title compound as a solid.

### Step D: 7-(tert-Butoxycarbonyl)-2-(trifluoromethyl)-3-vinyl-5,6,7,8-tetrahydroimidazo[1,2-a]pyrazine

To a solution of 400 mg (1.08 mmol) of 3-bromo-7-(tert-butoxycarbonyl)-2-(trifluoromethyl)-5,6,7,8-tetrahydroimidazo[1,2-a]pyrazine (Step C) and 686 mg (2.16 mmol) of tributyl(vinyl)tin in 25 mL of toluene was added 20 mg of dichlorobis(triphenylphosphine)palladium(II). After the reaction was heated at reflux temperature for 2 h, the mixture was cooled to ambient temperature and partitioned between dichloromethane and water. The aqueous phase was extracted with three portions of dichloromethane. The combined organic layers were washed with brine, dried over magnesium sulfate, and concentrated. The residue was purified by preparative TLC (silica gel, 25% ethyl acetate in hexanes) to give the title compound as a solid.

### Step E: 2-(Trifluoromethyl)-3-vinyl-5,6,7,8-tetrahydroimidazo[1,2-a]pyrazine

To 40.0 mg of 7-(tert-butoxycarbonyl)-2-(trifluoromethyl)-3-vinyl-5,6,7,8-tetrahydroimidazo[1,2-a]pyrazine (Step D) was added 1 mL of saturated methanolic hydrogen chloride. The reaction was stirred at ambient temperature for 1 h and concentrated to give 23.0 mg of the title compound as a viscous oil.

### Step F: 7-[(3R)-3-[(tert-Butoxycarbonyl)amino]-4-(2,5-difluorophenyl)butanoyl]-2-(trifluoromethyl)-3-vinyl-5,6,7,8-tetrahydroimidazo[1,2-a]pyrazine

The title compound was prepared from 2-(trifluoromethyl)-3-vinyl-5,6,7,8-tetrahydroimidazo[1,2-a]pyrazine (Step E) and (3R)-3-[(tert-butoxycarbonyl)amino]-4-(2,5-difluorophenyl)butanoic acid (intermediate 1) using a procedure analogous to that of Example 1, Step C. LC/MS 515 (M+1).

### Step G: 7-[(3R)-3-Amino-4-(2,5-difluorophenyl)butanoyl]-2-(trifluoromethyl)-3-vinyl-5,6,7,8-tetrahydroimidazo[1,2-a]pyrazine, hydrochloride

To 13.0 mg (0.0253 mmol) of 7-[(3R)-3-[(tert-butoxycarbonyl)amino]-4-(2,5-difluorophenyl)butanoyl]-2-(trifluoromethyl)-3-vinyl-5,6,7,8-tetrahydroimidazo[1,2-a]pyrazine (Step F) was added 1 mL of saturated methanolic hydrogen chloride. The reaction was stirred at ambient temperature for 1 h and concentrated to give the title compound as a solid. LC/MS 415 (M+1).

### EXAMPLE 14

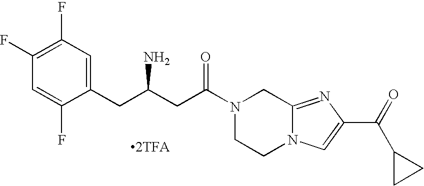

### [7-[(3R)-3-Amino-4-(2,4,5-trifluorophenyl)butanoyl]-5,6,7,8-tetrahydroimidazo[1,2-a]pyrazin-2-yl](cyclopropyl)methanone, bis-trifluoroacetic acid salt

### Step A: 7-(tert-Butoxycarbonyl)-2-[[methoxy(methyl)amino]carbonyl]-5,6,7,8-tetrahydroimidazo[1,2-a]pyrazine

To a solution of 3.40 g (11.5 mmol) of ethyl 7-(tert-butoxycarbonyl)-5,6,7,8-tetrahydroimidazo[1,2-a]pyrazine-2-carboxylate (Example 10, Step 1) in 60 mL of dichloromethane was added 8.64 mL (17.3 mmol) of 2N methoxy(methyl)amine, and then 1.35 g (13.8 mmol) of trimethylaluminum was added over 15 min. The reaction was stirred at ambient temperature for 14 h. The reaction was quenched slowly with water, the mixture was extracted with three portions of dichloromethane, and the combined organic layers were washed sequentially with 1N hydrochloric acid, saturated aqueous sodium bicarbonate solution and brine. The organic layer was dried over magnesium sulfate, and concentrated. The residue was purified by flash chromatography on a Biotage® system (silica gel, 100% ethyl acetate then 10% methanol in ethyl acetate) to give the title compound as a solid.

### Step B: 7-(tert-Butoxycarbonyl)-2-(cyclopropylcarbonyl)-5,6,7,8-tetrahydroimidazo[1,2-a]pyrazine

To a solution of 131 g (0.42 mmol) of 7-(tert-butoxycarbonyl)-2-[[methoxy(methyl)amino]carbonyl]-5,6,7,8-tetrahydroimidazo[1,2-a]pyrazine (Step A) in 6 mL of tetrahydrofuran (THF) at −40° C. was added 1.6 ml of 0.79M cyclopropylmagnesium bromide in THF dropwise. After the reaction was stirred at −40° C. for 40 min, the reaction mixture was quenched with 1.5 mL of cold 0.5N hydrochloric acid. The mixture was partitioned between ethyl acetate and water. The aqueous phase was extracted with three portions of ethyl acetate. The combined organic layers were washed with brine, dried over magnesium sulfate, and concentrated in vacuo. The residue was purified by preparative TLC (silica gel, 10% methanol in ethyl acetate) to give the title compound as a viscous oil. LC/MS 292 (M+1).

### Step C: Cyclopropyl (5,6,7,8-tetrahydroimidazo[1,2-a]pyrazin-2-yl)methanone

The title compound was prepared from 7-(tert-butoxycarbonyl)-2-(cyclopropylcarbonyl)-5,6,7,8-tetrahydroimidazo[1,2-a]pyrazine (Step B) using a procedure analogous to that of Example 13, Step E.

### Step D: [7-[(3R)-3-[(tert-Butoxycarbonylamino]-4-(2,4,5-trifluorophenyl)butanoyl]-5,6,7,8-tetrahydroimidazo[1,2-a]pyrazin-2-yl](cyclopropyl)methanone

The title compound was prepared from cyclopropyl (5,6,7,8-tetrahydroimidazo[1,2-a]pyrazin-2-yl)methanone (Step C) and (3R)-3-[(tert-butoxycarbonyl)amino]-4-(2,4,5-trifluorophenyl)butanoic acid (Intermediate 3) using a procedure analogous to that of Example 1, Step C. LC/MS 507 (M+1).

### Step E: [7-[(3R)-3-Amino-4-(2,4,5-trifluorophenyl)butanoyl]-5,6,7,8-tetrahydroimidazo[1,2-a]pyrazin-2-yl](cyclopropyl)methanone, bis-trifluoroacetic acid salt

The title compound was prepared from [7-[(3R)-3-[(tert-butoxycarbonyl)amino]-4-(2,4,5-trifluorophenyl)butanoyl]-5,6,7,8-tetrahydroimidazo[1,2-a]pyrazin-2-yl](cyclopropyl)methanone (Step D) using a procedure analogous to that of Example 1, Step D. LC/MS 407 (M+1).

### EXAMPLE 15

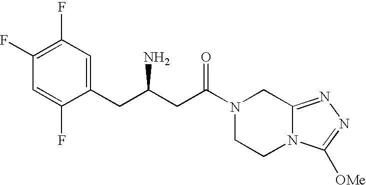

### 7-[(3R)-3-Amino-4-(2,4,5-trifluorophenyl)butanoyl]-3-methoxy-5,6,7,8-tetrahydro[1,2,4triazolo[4,3-a]pyrazine

### Step A: [1,2,4]Triazolo[4,3-a]pyrazine

A solution of 15.0 g (136 mmol) of 2-hydrazinopyrazine in 100 mL of trimethyl orthoformate was heated to reflux at 100° C. for 1.5 h. The solvent was removed in vacuo and the crude product was applied to a pad of silica gel and eluted with 15:85 methanol:ethyl acetate. The resulting solution was concentrated in vacuo to yield the title compound. LC/MS 121.0 (M+1).

### Step B: 5,6,7,8-Tetrahydro[1,2,4]triazolo[4,3-a]pyrazine

To a flask containing 9.15 g (76.2 mmol) of [1,2,4]triazolo[4,3-a]pyrazine was added 1.80 g of 10% palladium-on-carbon and the system was thoroughly degassed. To this was added 400 mL of methanol and the system was then evacuated and purged with hydrogen (1 atm) three times and then stirred under a positive hydrogen atmosphere at ambient temperature for 1 d. The crude reaction mixture was filtered through a pad of Celite and the filtrate was concentrated in vacuo to yield the title compound. LC/MS 125.0 (M+1).

### Step C: 7-(tert-Butoxycarbonyl)-5,6,7,8-tetrahydro[1,2,4]triazolo[4,3-a]pyrazine

To a solution of 9.70 g (76.2 mmol) of 5,6,7,8-tetrahydro[1,2,4]triazolo[4,3-a]pyrazine in 250 mL of dichloromethane was added sequentially 11.8 g (91.4 mmol) of N,N-diisopropylethylamine and 18.3 g (83.8 mmol) of di-tert-butyl dicarbonate. The reaction was stirred at ambient temperature for 2 h. The reaction mixture was then washed sequentially with saturated aqueous sodium bicarbonate solution and brine, dried over magnesium sulfate, and concentrated in vacuo. The residue was azeotroped with hexanes and the resultant solid triturated with a mixture of hexanes/ethyl acetate (95:5) to afford the title compound. LC/MS 225.1 (M+1).

### Step D: 3-Bromo-7-(tert-butoxycarbonyl)-5,6,7,8-tetrahydro[1,2,4]triazolo[4,3-a]pyrazine

To a solution of 9.01 g (40.0 mmol) of 7-(tert-butoxycarbonyl)-5,6,7,8-tetrahydro[1,2,4]triazolo[4,3-a]pyrazine in 150 mL chloroform was added 6.72 g (80.0 mmol) of sodium bicarbonate. The system was cooled to 0° C. and 7.11 g (40.0 mmol) of N-bromosuccinimide was added. The reaction was stirred at 0° C. for 15 min and at ambient temperature for 16 h. The reaction was diluted with dichloromethane and washed sequentially with saturated aqueous sodium bicarbonate solution and brine, dried over magnesium sulfate, and concentrated in vacuo. The crude product was purified by flash chromatography on a Biotage® system (silica gel, 4% methanol/ethyl acetate) to yield the title compound. LC/MS 303.0 and 305.0 (M+1).

### Step E: 7-(tert-Butoxycarbonyl)-3-methoxy-5,6,7,8-tetrahydro[1,2,4]triazolo[4,3-a]pyrazine

To a solution of 0.173 g (0.571 mmol) of 3-bromo-7-(tert-butoxycarbonyl)-5,6,7,8-tetrahydro[1,2,4]triazolo[4,3-a]pyrazine in 6 mL of methanol was added 0.39 mL of 25% w/w solution of sodium methoxide in methanol. The reaction was heated at 65° C. for 1 d. The reaction was diluted with ethyl acetate and washed sequentially with saturated aqueous sodium bicarbonate solution and brine, dried over magnesium sulfate, and concentrated in vacuo. The crude product was purified by flash chromatography on a Biotage® system (silica gel, 5% methanol/ethyl acetate) to yield the title compound. LC/MS 255.1 (M+1).

### Step F: 3-Methoxy-5,6,7,8-tetrahydro[1,2,4]triazolo[4,3-a]pyrazine, trifluoroacetic acid salt

A solution of 1:9 TFA/dichloromethane was added to 0.069 g (0.271 mmol) of 7-(tert-butoxycarbonyl)-3-methoxy-5,6,7,8-tetrahydro[1,2,4]triazolo[4,3-a]pyrazine at 0° C. and the reaction was stirred at 0° C. for 60 min and then at ambient temperature for 3 h. The reaction mixture was concentrated in vacuo to give the title compound. LC/MS 155.0 (M+1).

### Step G: 7-[(3R)-3-[(tert-Butoxycarbonyl)amino]-4-(2,4,5-trifluorophenyl)butanoyl]-3-methoxy-5,6,7,8-tetrahydro[1,2,4]triazolo[4,3-a]pyrazine

To a solution of 0.0727 g (0.271 mmol) of 3-methoxy-5,6,7,8-tetrahydro[1,2,4]triazolo[4,3-a]pyrazine, trifluoroacetic acid salt in 3 ml of dimethylformamide was added N,N-diisopropylethylamine until the pH of the reaction mixture was 9. This was followed by the sequential addition of 0.0902 g (0.271 mmol) of 3(R)-3-[(tert-butoxycarbonyl)amino]-4-(2,4,5-trifluorophenyl)butanoic acid (Intermediate 3), 0.0370 g (0.271 mmol) of HOAT and 0.100 g (0.271 mmol) of HATU, and the reaction was stirred at ambient temperature for 16 h. The reaction mixture was then diluted with ethyl acetate and washed sequentially with saturated aqueous sodium bicarbonate solution, 0.5N aqueous sodium bicarbonate solution, and brine, dried over magnesium sulfate and concentrated in vacuo. The crude product was purified by flash chromatography on a Biotage® system (silica gel, 15% methanol/ethyl acetate) to yield the title compound. LC/MS 470.1 (M+1).

### Step H: 7-[(3R)-3-Amino-4-(2,4,5-trifluorophenyl)butanoyl]-3-methoxy-5,6,7,8-tetrahydro[1,2,4]triazolo[4,3-a]pyrazine

1:9 TFA/dichloromethane (3 mL) was added to 0.0640 g (0.136 mmol) of 7-[(3R)-3-[(tert-butoxycarbonyl)amino]-4-(2,4,5 -trifluorophenyl)butanoyl]-3-methoxy-5,6,7,8-tetrahydro[1,2,4]triazolo[4,3-a]pyrazine at 0° C. The reaction was stirred at 0° C. for 15 min a at ambient temperature for 2.5 h. The reaction mixture was then diluted with ethyl acetate and washed sequentially with saturated aqueous sodium bicarbonate solution and brine, dried over magnesium sulfate, and concentrated in vacuo to afford the title compound. LC/MS 370.1 (M+1).

### EXAMPLE 16

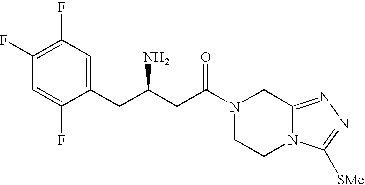

### 7-[(3R)-3-Amino-4-(2,4,5-trifluorophenyl)butanoyl]-3-(methylthio)-5,6,7,8-tetrahydro[1,2,4]triazolo[4,3-a]pyrazine

### Step A: 7-(tert-Butoxycarbonyl)-3-(methylthio)-5,6,7,8-tetrahydro[1,2,4]triazolo[4,3-a]pyrazine

To a solution of 0.090 g (0.297 mmol) of 3-bromo-7-(tert-butoxycarbonyl)-5,6,7,8-tetrahydro[1,2,4]triazolo[4,3-a]pyrazine (Example 15, Step D) in 3 mL of dioxane was added 0.0630 g (0.899 mmol) of sodium thiomethoxide, and the reaction mixture was heated at 100° C. for 1 d. The reaction was diluted with dichloromethane and washed sequentially with saturated aqueous sodium bicarbonate solution and brine, dried over magnesium sulfate, and concentrated in vacuo to afford the title compound. LC/MS 271.1 (M+1).

### Step B: 7-[(3R)-3-Amino-4-(2,4,5-trifluorophenyl)butanoyl]-3-(methylthio)-5,6,7,8-tetrahydro[1,2,4]triazolo[4,3-a]pyrazine

Using 7-(tert-butoxycarbonyl)-3-(methylthio)-5,6,7,8-tetrahydro[1,2,4]triazolo[4,3-a]pyrazine from Step A, the title compound was prepared essentially following the procedures outlines in Example 15, Steps E-H. LC/MS 386.3 (M+1).

### EXAMPLE 17

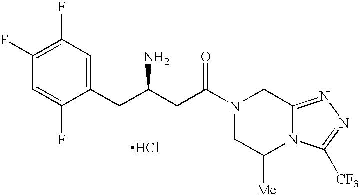

### 7-[(3R)-3-Amino-4-(2,4,5-trifluorophenyl)butanoyl]-5-methyl-3-(trifluoromethyl)-5,6,7,8-tetrahydro[1,2,4]triazolo[4,3-a]pyrazine, hydrochloride

### Step A: 2-Hydrazino-6-methylpyrazine

To 15 mL of hydrazine hydrate at ambient temperature was added dropwise 2.9 g of 2-chloro-6-methylpyrazine. The resultant mixture was placed in a pre-heated oil bath at ˜50° C. and then heated to −100° C. over 30 min. The mixture was allowed to cool to ambient temperature, and was then cooled in a refrigerator for 1 h. A portion of hydrazine hydrate was added to the solidified mixture, and the solid was collected by filtration. The filtrate was cooled in a refrigerator for 2 h, and a second crop was collected. The crops were combined and used without further purification. MS 124.9 (M+1).

### Step B: 5-Methyl-3-(trifluoromethyl)-1,2,4-triazolo[4,3-a]pyrazine

To 2.32 g (18.7 mmol) of the product from Step A was added 50 mL of trifluoroacetic anhydride that had been cooled to 0° C. The resultant mixture was stirred at ambient temperature for 1 h and concentrated in vacuo. To the resultant viscous material was added approximately 50 mL of polyphosphoric acid and the resultant mixture was heated at 120° C. for 18 h. The mixture was basified with ammonium hydroxide, and extracted with three portions of ethyl acetate. The combined organics were washed with brine, dried over magnesium sulfate, and concentrated in vacuo. The residue was purified by flash chromatography (silica gel, eluting sequentially with 50% and 100% ethyl acetate/hexane) to give the title compound. MS 203 (M+1).

### Step C: 5-Methyl-3-(trifluoromethyl)-5,6,7,8-tetrahydro-1,2,4-triazolo[4,3-a]pyrazine

To a solution of 720 mg (3.56 mmol) of the product from Step B in 10 mL of ethanol and 5 mL of tetrahydrofuran was added 60 mg of 10% palladium on activated carbon and the mixture stirred under 1 atmosphere of hydrogen at ambient temperature for 18 h. The solution was filtered through Celite and the filtrate concentrated in vacuo. The residue was purified by Biotage® flash chromatography (silica gel, eluting sequentially with ethyl acetate and 10% methanol/dichloromethane) to give the title compound as a colorless viscous oil. MS 207 (M+1).

### Step D: 7-[(3R)-3-Amino-4-(2,4,5-trifluorophenyl)butanoyl]-5-methyl-3-(trifluoromethyl)-5,6,7,8-tetrahydro-1,2,4-triazolo[4,3-a]pyrazine, hydrochloride

To a solution of 281 mg (1.36 mmol) of the product from Step C and 454 mg (1.36 mmol) of (3R)-3-[(1,1-dimethylethoxycarbonyl)amino]-4-(2,4,5-trifluorophenyl)butanoic acid in 2.5 mL of dimethylformamide was added 314 mg (1.64 mmol) of EDC. After stirring at ambient temperature for 18 h, the mixture was partitioned between ethyl acetate and aqueous sodium bicarbonate solution. The aqueous phase was extracted with three portions of ethyl acetate. The combined organics were washed with brine, dried over magnesium sulfate, and concentrated. The residue was purified by flash chromatography (silica gel, eluting sequentially with 50% and 100% ethyl acetate/hexane) to give the N-BOC-protected compound as a mixture of diastereomers. Chiral HPLC separation (ChiralCel OD column, 10% ethanol/hexane) provided the individual diastereomers, each of which were treated with methanolic hydrogen chloride at ambient temperature for 1 h. Concentration gave the individual diastereomers of the title compound. Faster eluting diastereomer: MS 422 (M+1); slower eluting diastereomer: MS 422 (M+1).

### EXAMPLE 18

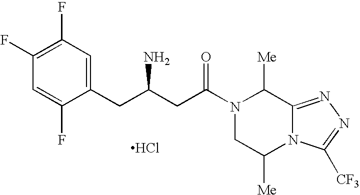

### (5S,8S)- and (5R,8R)-7-[(3R)-3-Amino-4-(2,4,5-trifluorophenyl)butanoyl]-5,8-dimethyl-3-(trifluoromethyl)-5,6,7,8-tetrahydro[1,2,4-triazolo[4,3-a]pyrazine, hydrochloride

### Step A: (5S,8S)- and (5R,8R)-5,8-Dimethyl-3-(trifluoromethyl)-5,6,7,8-tetrahydro-1,2,4-triazolo[4,3-a]pyrazine

The title compound, as a mixture of cis diastereomers, was prepared from 2-chloro-3,6-dimethylpyrazine essentially following the procedures outlined in Example 17, Steps A, B and C. MS 221 (M+1).

### Step B: (5S,8S)- and (5R,8R)-7-[(3R)-3-Amino-4-(2,4,5-trifluorophenyl)butanoyl]-5,8-dimethyl-3-(trifluoromethyl)-5,6,7,8-tetrahydro-1,2,4-triazolo[4,3-a]pyrazine

A solution of 1.0 g (4.6 mmol) of the product from Step A, 1.6 g (4.8 mmol) of (3R)-3-[(tert-butoxycarbonyl)amino]-4-(2,4,5-trifluorophenyl)butanoic acid (Intermediate 3), 740 mg (5.5 mmol) of HOAT, and 2.1 g (5.5 mmol) of HATU reagent in 20 mL of DMF was stirred at ambient temperature for 72 h and then concentrated in vacuo. The residue was partitioned between aqueous sodium bicarbonate solution and ethyl acetate. The aqueous phase was extracted with three portions of ethyl acetate. The combined organics were washed with brine, dried over magnesium sulfate, and concentrated in vacuo. Purification by flash chromatography (silica gel, gradient elution with 0-10% methanol/dichloromethane) gave impure product, which was resubjected to flash chromatography (silica gel, gradient elution with 5-50% ethyl acetate/hexanes) to afford the N-BOC compound as a mixture of diastereomers. Chiral HPLC separation (ChiralCel OD column, 10% ethanol/hexane) provided the individual diastereomers, each of which was treated with methanolic hydrogen chloride at ambient temperature for 30 min. Concentration gave the individual diastereomers of the title compound. Faster eluting diastereomer: MS 436 (M+1); slower eluting diastereomer: MS 436 (M+1).

### EXAMPLE 19

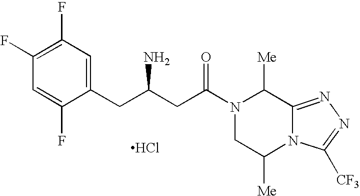

### (5S,8R)- and (5R,8S)-7-[(3R)-3-Amino-4-(2,4,5-trifluorophenyl)butanoyl]-5,8-dimethyl-3-(trifluoromethyl)-5,6,7,8-tetrahydro[1,2,4]triazolo[4,3-a]pyrazine, hydrochloride

### Step A: 7-(tert-Butoxycarbonyl)-5-methyl-3-(trifluoromethyl)-5,6,7,8-tetrahydro-1,2,4-triazolo[4,3-a]pyrazine

To a solution of 1.03 g (5.0 mmol) of 5-methyl-3-(trifluoromethyl)-5,6,7,8-tetrahydro-1,2,4-triazolo[4,3-a]pyrazine from Example 17, Step C in 10 mL of dichloromethane was added 1.09 g (5.0 mmol) of di-tert-butyldicarbonate. The mixture was stirred at ambient temperature for 2 h and concentrated. Purification by flash chromatography (silica gel, 50% ethyl acetate/hexane) gave the title compound as a white solid. MS 307 (M+1).

### Step B: 7-(tert-Butoxycarbonyl)-5,8-dimethyl-3-(trifluoromethyl)-5,6,7,8-tetrahydro-1,2,4-triazolo[4,3-a]pyrazine

To a solution of 1.0 g (3.3 mmol) of the compound from Step A in 14 mL of toluene at −78° C. was added 0.55 mL (3.6 mmol) of tetramethylethylenediamine followed by 2.5 mL (4.0 mmol) of n-butyllithium (1.6M in hexane). The resultant brown colored solution was stirred at −78° C. for 10 min and then 0.23 mL (3.6 mmol) of iodomethane was added dropwise. The mixture was stirred at −78° C. for 10 min, and then the cooling bath was removed. The mixture was stirred at ambient temperature for 2 h and then quenched by the addition of aqueous ammonium chloride solution. The mixture was extracted with three portions of ethyl acetate. The combined organics were washed with brine, dried over magnesium sulfate, and concentrated. Purification by preparative TLC (silica gel, 50% ethyl acetate) gave the title compound as a mixture of diastereomers. MS 321 (M+1).

### Step C: 5,8-Dimethyl-3-(trifluoromethyl)-5,6,7,8-tetrahydro-1,2,4-triazolo[4,3-a]pyrazine, hydrochloride

To a solution of the product from Step B in 1 mL of methanol was added 1 mL of saturated methanolic hydrogen chloride solution. The mixture was stirred at ambient temperature for 1 h. Concentration gave the title compound. MS 220.9 (M+1).

### Step D: (5S,8R)- and (5R,8S)-7-[(3R)-3-tert-Butoxycarbonylamino-4-(2,4,5-trifluorophenyl)butanoyl]-5,8-dimethyl-3-(trifluoromethyl)-5,6,7,8-tetrahydro[1,2,4triazolo[4,3-a]pyrazine

To a solution of 35 mg (0.14 mmol) of product from Step C and 45 mg (0.14 mmol) of (3R)-3-[(tert-butoxycarbonyl)amino]-4-(2,4,5-trifluorophenyl)butanoic acid (Intermediate 3) in 1 mL of DMF was added 0.028 mL (0.16 mmol) of N,N-diisopropylethylamine, 22 mg (0.16 mmol) of HOAT, and 62 mg (0.16 mmol) of HATU reagent. After 18 h at ambient temperature, the reaction mixture was concentrated. The residue was partitioned between aqueous sodium bicarbonate solution and ethyl acetate. The aqueous phase was extracted with three portions of ethyl acetate. The combined organics were washed with brine, dried over magnesium sulfate, and concentrated in vacuo. Purification by preparative TLC (silica gel, ethyl acetate) afforded the title compound as a mixture of four diastereomers. Chiral HPLC separation (ChiralCel OD column, 7% ethanol/hexane) provided the individual diastereomers, the fastest and slowest eluting of which were the desired trans isomers. Fastest eluting diastereomer: MS 536 (M+1); slowest eluting diastereomer: MS 536 (M+1).

### Step E: (5S,8R)- and (5R,8S)-7-[(3R)-3-Amino-4-(2,4,5-trifluorophenyl)butanoyl-5,8-dimethyl-3-(trifluoromethyl)-5,6,7,8-tetrahydro[1,2,4]triazolo[4,3-a]pyrazine hydrochloride

The individual diastereomers from Step D were treated separately with methanolic hydrogen chloride at ambient temperature for 1 h. Concentration gave the title compounds. From fastest eluting diastereomer: MS 436 (M+1); from slowest eluting diastereomer: MS 436 (M+1).

### EXAMPLE 20

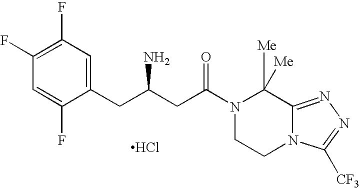

### 7-[(3R)-3-Amino-4-(2,4,5-trifluorophenyl)butanoyl]-8,8-dimethyl-3-(trifluoromethyl)-5,6,7,8-tetrahydro1,2,4triazolo[4,3-a]pyrazine, hydrochloride

### Step A: 3-(Trifluoromethyl)-1,2,4-triazolo[4,3-a]pyrazine

A mixture of 2-hydrazinopyrazine (820 mg, 7.45 mmol), prepared from 2-chloropyrazine and hydrazine using a procedure analogous to that described in the literature (P. J. Nelson and K. T. Potts, J. Org. Chem. 1962, 27, 3243, except that the crude product was extracted into 10% methanol/dichloromethane and filtered, and the filtrate was concentrated and purified by flash chromatography on silica gel, eluting with 100% ethyl acetate followed by 10% methanol in dichloromethane), TFA (2.55 g, 22.4 mmol), and polyphosphoric acid (10 mL) was heated to 140° C. with stirring for 18 h. The solution was added to ice and neutralized by the addition of ammonium hydroxide. The aqueous solution was extracted with ethyl acetate (3×), washed with brine, and dried over anhydrous magnesium sulfate. Concentration followed by flash chromatography (silica gel, 1:1 hexane:ethyl acetate, then 100% ethyl acetate) afforded the title compound as a solid. ¹H NMR (500 MHz, CDCl₃) δ 8.17-8.20 (m, 2H), 9.54 (s, 1H). LC/MS (M+1) 189.

### Step B: 3-(Trifluoromethyl)-5,6,7,8-tetrahydro-1,2,4-triazolo[4,3-a]pyrazine

3-(Trifluoromethyl)-1,2,4-triazolo[4,3-a]pyrazine (540 mg, 2.87 mmol, from Step A) was hydrogenated under atmospheric hydrogen with 10% Pd/C (200 mg) as a catalyst in ethanol (10 mL) at ambient temperature for 18 h. Filtration through Celite followed by concentration gave a dark colored oil. Dichloromethane was added to the above oil and the insoluble black precipitate was filtered off. Concentration of the filtrate gave the title compound as an oil. ¹H NMR (500 MHz, CDCl₃) δ 2.21 (br, 1H), 3.29 (t, 2H, J=5.5 Hz), 4.09 (t, 2H, J=5.5 Hz), 4.24 (s, 2H). MS (M+1) 193.

### Step C: 7-tert-Butoxycarbonyl-3-(trifluoromethyl)-5,6,7,8-tetrahydro-1,2,4-triazolo[4,3-a]pyrazine

The product from Step B was converted to the title compound essentially following the procedure outlined in Example 19, Step A.

### Step D: 7-tert-Butoxycarbonyl-8-methyl-3-(trifluoromethyl)-5,6,7,8-tetrahydro-1,2,4-triazolo[4,3-a]pyrazine

The product from Step C was alkylated with methyl iodide, essentially following the procedure outlined in Example 19, Step B. Purification by flash chromatography (silica gel, eluting sequentially with 20% and 50% ethyl acetate/hexane) gave the title compound. MS 307 (M+1).

### Step E: 7-tert-Butoxycarbonyl-8,8-dimethyl-3-(trifluoromethyl)-5,6,7,8-tetrahydro-1,2,4-triazolo[4,3-a]pyrazine

The product from Step D was alkylated a second time with methyl iodide, essentially following the procedure outlined in Example 19, Step B. Purification by Biotage® flash chromatography (silica gel, 20% ethyl acetate/hexane) gave the title compound. MS 321 (M+1).

### Step F: 8,8-Dimethyl-3-(trifluoromethyl)-5,6,7,8-tetrahydro-1,2,4-triazolo[4,3-a]pyrazine

To a solution of 224 mg of the product from Step E in 2 mL of methanol was added 2 mL of saturated methanolic hydrogen chloride solution. The mixture was stirred at ambient temperature for 1 h and then concentrated. Purification by preparative TLC (silica gel, 80:15:1 dichloromethane/methanol/ammonium hydroxide) gave the title compound. MS 220.9 (M+1).

### Step G: 7-[(3R)-3-tert-Butoxycarbonylamino-4-(2,4,5-trifluorophenyl)butanoyl]-8,8-dimethyl-3-(trifluoromethyl)-5,6,7,8-tetrahydro[1,2,4triazolo[4,3-a]pyrazine

The title compound was prepared essentially following the procedure outlined in Example 18, Step B, stirring the reaction mixture for 18 h prior to work-up. Purification by preparative TLC (silica gel, ethyl acetate) provided the title compound. MS 480 (M+1-tBu), 536 (M+1).

### Step H: 7-[(3R)-Amino-4-(2,4,5-trifluorophenyl)butanoyl]-8,8-dimethyl-3-(trifluoromethyl)-5,6,7,8-tetrahydro[1,2,4triazolo4,3-a]pyrazine, hydrochloride

A solution of 10 mg of the product from Step G in 0.5 mL of methanol and 0.5 mL of saturated methanolic hydrogen chloride was stirred at ambient temperature for 1 h. Concentration gave the title compound. MS 436 (M+1).

### EXAMPLE 21

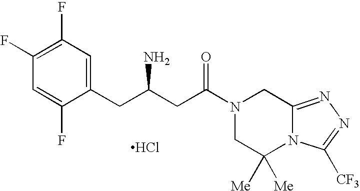

### 7-[(3R)-3-Amino-4-(2,4,5-trifluorophenyl)butanoyl]-5,5-dimethyl-3-(trifluoromethyl)-5,6,7,8-tetrahydro[1,2,4]triazolo[4,3-a]pyrazine, hydrochloride

### Step A: 5,5-Dimethyl-3-(trifluoromethyl)-5,6,7,8-tetrahydro-1,2,4-triazolo[4,3-a]pyrazine

To a solution of 2.17 g (11.7 mmol) of 2-(chloromethyl)-5-(trifluoromethyl)-1,3,4-oxadiazole in 10 mL of methanol at 0° C. was added 1.23 mL (11.7 mmol) of 2-methylpropane-1,2-diamine followed by 2.04 mL (11.7 mmol) of N,N-diisopropylethylamine. The reaction mixture was stirred at 0° C. for 30 min and then at ambient temperature for 2 h. A white precipitate formed. The mixture was filtered. The filtrate was concentrated to give a viscous oil, which was dissolved in superphosphoric acid and heated at 110° C. for 18 h. After cooling to ambient temperature, the reaction mixture was poured onto ice and made basic by the addition of ammonium hydroxide. The mixture was extracted with three portions of ethyl acetate. The combined organics were washed with brine, dried over magnesium sulfate, and concentrated. Purification by Biotage® flash chromatography (silica gel, eluting sequentially with 10% methanol/dichloromethane and 80:15:1 dichloromethane:methanol:ammonium hydroxide) gave the title compound as a semi-solid. MS 220.9 (M+1).

### Step B: 7-[(3R)-3-Amino-4-(2,4,5-trifluorophenyl)butanoyl]-5,5-dimethyl-3-(trifluoromethyl)-5,6,7,8-tetrahydro[1,2,4]triazolo4,3-a]pyrazine, hydrochloride

The product from Step A was coupled to (3R)-3-[(1,1-dimethylethoxycarbonyl)amino]-4-(2,4,5-trifluorophenyl)butanoic acid, essentially following the procedure outlined in Exmple 17, Step D. Purification of the N-BOC intermediate by preparative TLC (silica gel, ethyl acetate) followed by deprotection with methanolic hydrogen chloride gave the title compound. MS 436 (M+1).

Essentially following the procedures outlined for Examples 1-21, the compounds listed in Table 2 were prepared.

TABLE 2

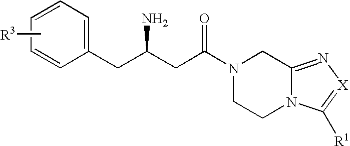

| | | | | MS |
|---|---|---|---|---|
| Ex. | R³ | R¹ | X | (M + 1) |
| 22 | 2-F,5-F | H | C-(morpholin-4- | 434 |
| | | | ylcarbonyl) | |
| 23 | 2-F,4-F,5-F | H | C-(morpholin-4- | 452 |
| | | | ylcarbonyl) | |
| 24 | 2-F,5-F | H | C—CON(tert-Bu) | 420 |
| 25 | 2-F,4-F,5-F | H | C—CON(tert-Bu) | 438 |
| 26 | 2-F,5-F | Cl | C—COOEt | 427.1 |
| 27 | 2-F,5-F | Cl | C—COOH | 399.1 |
| 28 | 2-F,4-F,5-F | Cl | C—COOH | 417.5 |
| 29 | 2-F,5-F | H | C—CONH— | 432 |
| | | | (tetrazol-5-yl) | |
| 30 | 2-F,4-F,5-F | H | C—CONH— | 450 |
| | | | (tetrazol-5-yl) | |
| 31 | 2-F,4-F,5-F | OCHMe₂ | N | 398.4 |
| 32 | 2-F,4-F,5-F | NHCOCH₃ | C-cyclopropyl | 436 |
| 33 | 2-F,4-F,5-F | NHCHMe₂ | C-cyclopropyl | 436 |
| 34 | 2-F,5-F | Cl | C—CF₃ | 423 |
| 35 | 2-F,5-F | Br | C—CF₃ | 467, |
| | | | | 469 |

### EXAMPLE OF A PHARMACEUTICAL FORMULATION

As a specific embodiment of an oral pharmaceutical composition, a 100 mg potency tablet is composed of 100 mg of any of the compounds of the present invention, 268 mg microcrystalline cellulose, 20 mg of croscarmellose sodium, and 4 mg of magnesium stearate. The active, microcrystalline cellulose, and croscarmellose are blended first. The mixture is then lubricated by magnesium stearate and pressed into tablets.

While the invention has been described and illustrated with reference to certain particular embodiments thereof, those skilled in the art will appreciate that various adaptations, changes modifications, substitutions, deletions, or additions of procedures and protocols may be made without departing from the spirit and scope of the invention. For example, effective dosages other than the particular dosages as set forth herein above may be applicable as a consequence of variations in responsiveness of the mammal being treated for any of the indications with the compounds of the invention indicated above. The specific pharmacological responses observed may vary according to and depending upon the particular active compounds selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims which follow and that such claims be interpreted as broadly as is reasonable.

1. A compound of structural formula I: 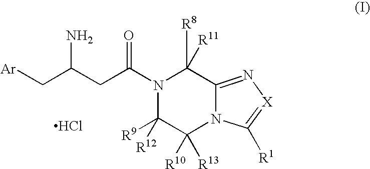
or a pharmaceutically acceptable salt thereof; wherein
each n is independently 0, 1, or 2;
X is N or CR²;
Ar is phenyl substituted with one to five R³ substituents;
R¹ and R² are each independently selected from the group consisting of
hydrogen,
halogen,
hydroxy,
cyano,
C₁₋₁₀ alkyl, wherein alkyl is unsubstituted or substituted with one to five substituents independently selected from halogen or hydroxy,
C₁₋₁₀ alkoxy, wherein alkoxy is unsubstituted or substituted with one to five substituents independently selected from halogen or hydroxy,
C₁₋₁₀ alkylthio, wherein alkylthio is unsubstituted or substituted with one to five substituents independently selected from halogen or hydroxy,
C₂₋₁₀ alkenyl, wherein alkenyl is unsubstituted or substituted with one to five substituents independently selected from halogen or hydroxy,
(CH₂)ₙCOOH,
(CH₂)ₙCOOC₁₋₆ alkyl,
(CH₂)ₙCONR⁴R⁵, wherein R⁴ and R⁵ are independently selected from the group consisting of hydrogen, tetrazolyl, thiazolyl, (CH₂)ₙ-phenyl, (CH₂)ₙ—C₃₋₆ cycloalkyl, and C₁₋₆ alkyl, wherein alkyl is unsubstituted or substituted with one to five halogens and wherein phenyl and cycloalkyl are unsubstituted or substituted with one to five substituents independently selected from halogen, hydroxy, C₁₋₆ alkyl, and C₁₋₆ alkoxy, wherein alkyl and alkoxy are unsubstituted or substituted with one to five halogens;
or R⁴ and R⁵ together with the nitrogen atom to which they are attached form a heterocyclic ring selected from azetidine, pyrrolidine, piperidine, piperazine, and morpholine wherein said heterocyclic ring is unsubstituted or substituted with one to five substituents independently selected from halogen, hydroxy, C₁₋₆ alkyl, and C₁₋₆ alkoxy, wherein alkyl and alkoxy are unsubstituted or substituted with one to five halogens;
(CH₂)ₙ—NR⁴R⁵,
(CH₂)ₙ—OCONR⁴R⁵,
(CH₂)ₙ—SO₂NR⁴R⁵,
(CH₂)ₙ—SO₂R⁶,
(CH₂)ₙ—NR⁷SO₂R⁶,
(CH₂)ₙ—NR⁷CONR⁴R⁵,
(CH₂)ₙ—NR⁷COR⁷,
(CH₂)ₙ—NR⁷CO₂R⁶,
(CH₂)ₙ—COR⁶,
(CH₂)ₙ—C₃₋₆ cycloalkyl, wherein cycloalkyl is unsubstituted or substituted with one to three substituents independently selected from halogen, hydroxy, C₁₋₆ alkyl, and C₁₋₆ alkoxy, wherein alkyl and alkoxy are unsubstituted or substituted with one to five halogens,
(CH₂)ₙ-aryl, wherein aryl is unsubstituted or substituted with one to five substituents independently selected from halogen, cyano, hydroxy, NR⁷SO₂R⁶, SO₂R⁶, CO₂H, C₁₋₆ alkyloxycarbonyl, C₁₋₆ alkyl, and C₁₋₆ alkoxy, wherein alkyl and alkoxy are unsubstituted or substituted with one to five halogens,
(CH₂)ₙ-heteroaryl, wherein heteroaryl is unsubstituted or substituted with one to three substituents independently selected from hydroxy, halogen, C₁₋₆ alkyl, and C₁₋₆ alkoxy, wherein alkyl and alkoxy are unsubstituted or substituted with one to five halogens, and
(CH₂)ₙ-heterocyclyl, wherein heterocyclyl is unsubstituted or substituted with one to three substituents independently selected from oxo, hydroxy, halogen, C₁₋₆ alkyl, and C₁₋₆ alkoxy, wherein alkyl and alkoxy are unsubstituted or substituted with one to five halogens,
wherein any methylene (CH₂) carbon atom in R¹ or R² is unsubstituted or substituted with one to two groups independently selected from halogen, hydroxy, and C₁₋₄ alkyl unsubstituted or substituted with one to five halogens;
each R³ is independently selected from the group consisting of
hydrogen,
halogen,
cyano,
hydroxy,
C₁₋₆ alkyl, unsubstituted or substituted with one to five halogens, and
C₁₋₆ alkoxy, unsubstituted or substituted with one to five halogens;
R⁶ is independently selected from the group consisting of tetrazolyl, thiazolyl, (CH₂)ₙ-phenyl, (CH₂)ₙ—C₃₋₆ cycloalkyl, and C₁₋₆ alkyl, wherein alkyl is unsubstituted or substituted with one to five halogens and wherein phenyl and cycloalkyl are unsubstituted or substituted with one to five substituents independently selected from halogen, hydroxy, C₁₋₆ alkyl, and C₁₋₆ alkoxy, wherein alkyl and alkoxy are unsubstituted or substituted with one to five halogens, and wherein any methylene (CH₂) carbon atom in R⁶ is unsubstituted or substituted with one to two groups independently selected from halogen, hydroxy, C₁₋₄ alkyl, and C₁₋₄ alkoxy, wherein alkyl and alkoxy are unsubstituted or substituted with one to five halogens;
each R⁷ is hydrogen or R⁶;
R⁸, R⁹ and R¹⁰ are each independently selected from the group consisting of
hydrogen,
cyano,
carboxy,
C₁₋₆ alkyloxycarbonyl,
C₁₋₁₀ alkyl, unsubstituted or substituted with one to five substituents independently selected from halogen, hydroxy, C₁₋₆ alkoxy, carboxy, C₁₋₆ alkyloxycarbonyl, and phenyl-C₁₋₃ alkoxy, wherein alkoxy is unsubstituted or substituted with one to five halogens,
(CH₂)ₙ-aryl, wherein aryl is unsubstituted or substituted with one to five substituents independently selected from halogen, hydroxy, C₁₋₆ alkyl, and C₁₋₆ alkoxy, wherein alkyl and alkoxy are unsubstituted or substituted with one to five halogens,
(CH₂)ₙ-heteroaryl, wherein heteroaryl is unsubstituted or substituted with one to three substituents independently selected from hydroxy, halogen, C₁₋₆ alkyl, and C₁₋₆ alkoxy, wherein alkyl and alkoxy are unsubstituted or substituted with one to five halogens,
(CH₂)ₙ-heterocyclyl, wherein heterocyclyl is unsubstituted or substituted with one to three substituents independently selected from oxo, hydroxy, halogen, C₁₋₆ alkyl, and C₁₋₆ alkoxy, wherein alkyl and alkoxy are unsubstituted or substituted with one to five halogens,
(CH₂)ₙ—C₃₋₆ cycloalkyl, wherein cycloalkyl is unsubstituted or substituted with one to three substituents independently selected from halogen, hydroxy, C₁₋₆ alkyl, and C₁₋₆ alkoxy, wherein alkyl and alkoxy are unsubstituted or substituted with one to five halogens, and
(CH₂)ₙCONR⁴R⁵, wherein R⁴ and R⁵ are independently selected from the group consisting of hydrogen, tetrazolyl, thiazolyl, (CH₂)ₙ-phenyl, (CH₂)ₙ—C₃₋₆ cycloalkyl, and C₁₋₆ alkyl, wherein alkyl is unsubstituted or substituted with one to five halogens and wherein phenyl and cycloalkyl are unsubstituted or substituted with one to five substituents independently selected from halogen, hydroxy, C₁₋₆ alkyl, and C₁₋₆ alkoxy, wherein alkyl and alkoxy are unsubstituted or substituted with one to five halogens;
or R⁴ and R⁵ together with the nitrogen atom to which they are attached form a heterocyclic ring selected from azetidine, pyrrolidine, piperidine, piperazine, and morpholine wherein said heterocyclic ring is unsubstituted or substituted with one to five substituents independently selected from halogen, hydroxy, C₁₋₆ alkyl, and C₁₋₆ alkoxy, wherein alkyl and alkoxy are unsubstituted or substituted with one to five halogens;
wherein any methylene (CH₂) carbon atom in R⁸, R⁹ or R¹⁰ is unsubstituted or substituted with one to two groups independently selected from halogen, hydroxy, and C₁₋₄ alkyl unsubstituted or substituted with one to five halogens;
with the proviso that when X is N, R¹⁰ , R¹¹, R¹² and R¹³ are hydrogen,
R⁸ or R⁹ is
hydrogen;
cyano;
C₁₋₁₀ alkyl, unsubstituted or substituted with one to five substituents selected from:
(1) halogen,
(2) hydroxy,
(3) phenyl, optionally substituted with one to five substituents independently selected from halogen, hydroxy, C₁₋₆ alkyl, and C₁₋₆ alkoxy, wherein alkyl and alkoxy are optionally substituted with one to five halogens,
(4) naphthyl, optionally substituted with one to five substituents independently selected from halogen, hydroxy, C₁₋₆ alkyl, and C₁₋₆ alkoxy, wherein alkyl and alkoxy are optionally substituted with one to five halogens,
(5) CO₂H,
(6) CO₂C₁₋₆ alkyl,
(7) CONR¹¹R¹², wherein R¹¹ and R¹² are independently selected from the group consisting of hydrogen, tetrazolyl, phenyl, C₃₋₆ cycloalkyl and C₁₋₆ alkyl, wherein alkyl is optionally substituted with one to six substituents independently selected from halogen and phenyl, wherein the phenyl or C₃₋₆ cycloalkyl being R¹¹ or R¹² or the optional phenyl substituent on C₁₋₆ alkyl are optionally substituted with one to five substituents independently selected from halogen, hydroxy, C₁₋₆ alkyl, and C₁₋₆ alkoxy, said C₁₋₆ alkyl and C₁₋₆ alkoxy being optionally substituted with one to five halogens,
or wherein R¹¹ and R¹² are optionally joined to form a ring selected from pyrrolidine, piperidine and morpholine;
phenyl, which is unsubstituted or substituted with one to five substituents independently selected from C₁₋₆ alkyl, C₁₋₆ alkoxy, hydroxy, and halogen, wherein alkyl and alkoxy are optionally substituted with one to five halogens;
naphthyl, which is unsubstituted or substituted with one to five substituents independently selected from C₁₋₆ alkyl, C₁₋₆ alkoxy, hydroxy, and halogen, wherein alkyl and alkoxy are optionally substituted with one to five halogens;
CO₂H;
C₁₋₆ alkyloxycarbonyl;
CONR¹¹R¹²; or
C₃₋₆ cycloalkyl, which is optionally substituted with one to five substituents independently selected from halogen, hydroxy, C₁₋₆ alkyl, and C₁₋₆ alkoxy, wherein alkyl and alkoxy are optionally substituted with one to five halogens; and when X is CR² and
R² is
hydrogen, cyano,
C₁₋₁₀ alkyl, unsubstituted or substituted with one to five halogens,
(CH₂)ₙ-phenyl, which is unsubstituted or substituted with one to five substituents independently selected from halogen, cyano hydroxy, R¹³, OR¹³, NHSO₂R¹³, SO₂R¹³, CO₂H, and C₁₋₆ alkyloxycarbonyl, wherein R¹³ is C₁₋₆ alkyl, unsubstituted or substituted with one to five halogens; or
a 5- or 6-membered heterocycle which may be saturated or unsaturated comprising one to four heteroatoms independently selected from N, S and O, the heterocycle being unsubstituted or substituted with one to three substituents independently selected from oxo, hydroxy, halogen,
C₁₋₆ alkyl, and C₁₋₆ alkoxy, wherein alkyl and alkoxy are optionally substituted with one to five halogens;
then in both cases R¹ is not
(1) hydrogen,
(2) cyano,
(3) C₁₋₁₀ alkyl, unsubstituted or substituted with one to five halogens,
(4) (CH₂)ₙ-phenyl, which is unsubstituted or substituted with one to five substituents independently selected from halogen, cyano hydroxy, R¹³, OR¹³, NHSO₂R¹³, SO₂R¹³, CO₂H, and C₁₋₆ alkyloxycarbonyl, wherein R¹³ is C₁₋₆ alkyl, unsubstituted or substituted with one to five halogens; or
(5) a 5- or 6-membered heterocycle which may be saturated or unsaturated comprising one to four heteroatoms independently selected from N, S and O, the heterocycle being unsubstituted or substituted with one to three substituents independently selected from oxo, hydroxy, halogen,
C₁₋₆ alkyl, and C₁₋₆ alkoxy, wherein alkyl and alkoxy are optionally substituted with one to five halogens; and
R¹¹, R¹² and R¹³ are each independently hydrogen or C₁₋₆ alkyl.

2. The compound of claim 1 of the structural formula Ia wherein the carbon atom marked with an * has the S configuration

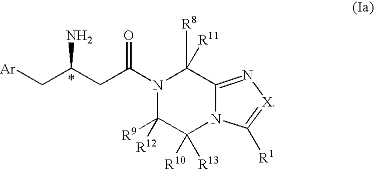

3. The compound of claim 1 of the structural formula Ib

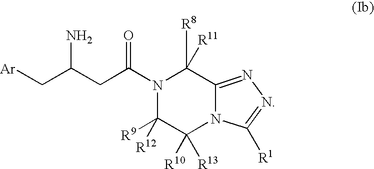

4. The compound of claim 3 of the structural formula Ic wherein the carbon atom marked with an * has the R configuration

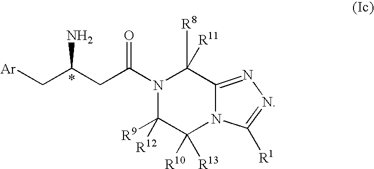

5. The compound of claim 3 of the structural formula Id:

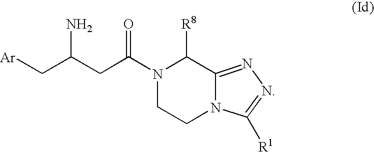

6. The compound of claim 5 wherein R⁸ is hydrogen.

7. The compound of claim 1 of the structural formula Ie

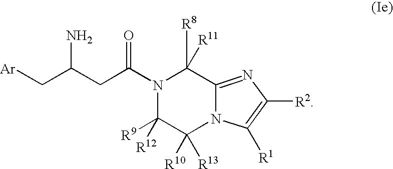

8. The compound of claim 7 of the structural formula If wherein the carbon atom marked with an * has the R configuration

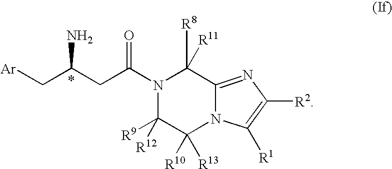

9. The compound of claim 7 of the structural formula Ig

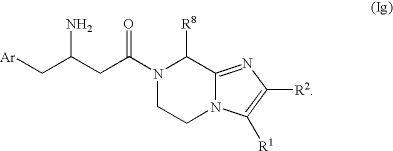

10. The compound of claim 9 wherein R⁸ is hydrogen.

11. The compound of claim 1 wherein R³ is selected from the group consisting of hydrogen, fluoro, chloro, bromo, trifluoromethyl, and methyl.

12. The compound of claim 11 wherein R³ is selected from the group consisting of hydrogen, fluoro, and chloro.

13. The compound of claim 1 wherein R¹ is selected from the group consisting of
hydrogen,
halogen,
C₁₋₆ alkyl, wherein alkyl is unsubstituted or substituted with one to five substituents independently selected from halogen or hydroxy,
C₁₋₆ alkoxy, wherein alkoxy is unsubstituted or substituted with one to five substituents independently selected from halogen or hydroxy,
C₁₋₆ alkylthio, wherein alkylthio is unsubstituted or substituted with one to five substituents independently selected from halogen or hydroxy,
C₂₋₆ alkenyl, wherein alkenyl is unsubstituted or substituted with one to five substituents independently selected from halogen or hydroxy,
(CH₂)ₙCOOH,
(CH₂)ₙCOOC₁₋₆ alkyl,
(CH₂)ₙCONR⁴R⁵, wherein R⁴ and R⁵ are independently selected from the group consisting of hydrogen, tetrazolyl, thiazolyl, (CH₂)ₙ-phenyl, (CH₂)ₙ—C₃₋₆ cycloalkyl, and C₁₋₆ alkyl, wherein alkyl is unsubstituted or substituted with one to five halogens and wherein phenyl and cycloalkyl are unsubstituted or substituted with one to five substituents independently selected from halogen, hydroxy, C₁₋₆ alkyl, and C₁₋₆ alkoxy, wherein alkyl and alkoxy are unsubstituted or substituted with one to five halogens;
or R⁴ and R⁵ together with the nitrogen atom to which they are attached form a heterocyclic ring selected from azetidine, pyrrolidine, piperidine, piperazine, and morpholine wherein said heterocyclic ring is unsubstituted or substituted with one to five substituents independently selected from halogen, hydroxy, C₁₋₆ alkyl, and C₁₋₆ alkoxy, wherein alkyl and alkoxy are unsubstituted or substituted with one to five halogens,
(CH₂)ₙ—NR⁴R⁵,
(CH₂)ₙ—NR⁷COR⁷,
(CH₂)ₙ—C₃₋₆ cycloalkyl, wherein cycloalkyl is unsubstituted or substituted with one to three substituents independently selected from halogen, hydroxy, C₁₋₆ alkyl, and C₁₋₆ alkoxy, wherein alkyl and alkoxy are unsubstituted or substituted with one to five halogens, and
(CH₂)ₙ-aryl, wherein aryl is unsubstituted or substituted with one to five substituents independently selected from halogen, CN, hydroxy, NR⁷SO₂R⁶, SO₂R⁶, CO₂H, C₁₋₆ alkyloxycarbonyl, C₁₋₆ alkyl, and C₁₋₆ alkoxy, wherein alkyl and alkoxy are unsubstituted or substituted with one to five halogens;
wherein any methylene (CH₂) carbon atom in R¹ or R² is unsubstituted or substituted with one to two groups independently selected from halogen, hydroxy, and C₁₋₄ alkyl unsubstituted or substituted with one to five halogens.

14. The compound of claim 13 wherein R¹ is selected from the group consisting of
hydrogen,
methyl,
ethyl,
trifluoromethyl,
CH₂CF₃,
CF₂CF₃,
phenyl,
cyclopropyl,
fluoro,
chloro,
bromo,
vinyl,
amino,
isopropylamino,
acetylamino,
2,2,2-trifluoroacetylamino,
tert-butylaminocarbonyl,
ethoxycarbonyl,
carboxy,
b 1-hydroxyethyl,
methoxy,
isopropoxy, and
methylthio.

15. The compound of claim 1 wherein R² is selected from the group consisting of
R² is selected from the group consisting of
hydrogen,
halogen,
C₁₋₆ alkyl, wherein alkyl is unsubstituted or substituted with one to five substituents independently selected from halogen or hydroxy,
C₂₋₆ alkenyl, wherein alkenyl is unsubstituted or substituted with one to five substituents independently selected from halogen or hydroxy,
(CH₂)ₙCOOH,
(CH₂)ₙCOOC₁₋₆ alkyl,
(CH₂)ₙCONR⁴R⁵, wherein R⁴ and R⁵ are independently selected from the group consisting of hydrogen, tetrazolyl, thiazolyl, (CH₂)ₙ-phenyl, (CH₂)ₙ—C₃₋₆ cycloalkyl, and C₁₋₆ alkyl, wherein alkyl is unsubstituted or substituted with one to five halogens and wherein phenyl and cycloalkyl are unsubstituted or substituted with one to five substituents independently selected from halogen, hydroxy, C₁₋₆ alkyl, and C₁₋₆ alkoxy, wherein alkyl and alkoxy are unsubstituted or substituted with one to five halogens;
or R⁴ and R⁵ together with the nitrogen atom to which they are attached form a heterocyclic ring selected from azetidine, pyrrolidine, piperidine, piperazine, and morpholine wherein said heterocyclic ring is unsubstituted or substituted with one to five substituents independently selected from halogen, hydroxy, C₁₋₆ alkyl, and C₁₋₆ alkoxy, wherein alkyl and alkoxy are unsubstituted or substituted with one to five halogens,
(CH₂)ₙ—NR⁴R⁵,
(CH₂)ₙ—NR⁷COR⁷,
(CH₂)ₙ—COR⁶,
(CH₂)ₙ—C₃₋₆ cycloalkyl, wherein cycloalkyl is unsubstituted or substituted with one to three substituents independently selected from halogen, hydroxy, C₁₋₆ alkyl, and C₁₋₆ alkoxy, wherein alkyl and alkoxy are unsubstituted or substituted with one to five halogens, and
(CH₂)ₙ-aryl, wherein aryl is unsubstituted or substituted with one to five substituents independently selected from halogen, CN, hydroxy, NR⁷SO₂R⁶, SO₂R⁶, CO₂H, C₁₋₆ alkyloxycarbonyl, C₁₋₆ alkyl, and
C₁₋₆ alkoxy, wherein alkyl and alkoxy are unsubstituted or substituted with one to five halogens;
wherein any methylene (CH₂) carbon atom in R¹ or R² is unsubstituted or substituted with one to two groups independently selected from halogen, hydroxy, and C₁₋₄ alkyl unsubstituted or substituted with one to five halogens.

16. The compound of claim 15 wherein R² is selected from the group consisting of
hydrogen
trifluoromethyl,
phenyl,
cyclopropyl,
carboxy,
ethoxycarbonyl,
dimethylaminocarbonyl,
aminocarbonyl,
morpholin-4-ylcarbonyl,
tert-butylaminocarbonyl,
cyclopropylcarbonyl,
tetrazol-5-ylaminocarbonyl, and
2,2,2-trifluoroacetylamino.

17. The compound of claim 1 wherein R⁸, R⁹, and R¹⁰ are each independently selected from the group consisting of
hydrogen,
C₁₋₆ alkyl, unsubstituted or substituted with one to five substituents independently selected from halogen, hydroxy, C₁₋₆ alkoxy, and phenyl-C₁₋₃ alkoxy, wherein alkoxy is unsubstituted or substituted with one to five halogens,
(CH₂)ₙ-phenyl, wherein phenyl is unsubstituted or substituted with one to five substituents independently selected from halogen, hydroxy, C₁₋₆ alkyl, and C₁₋₆ alkoxy, wherein alkyl and alkoxy are unsubstituted or substituted with one to five halogens,
(CH₂)ₙ-heteroaryl, wherein heteroaryl is unsubstituted or substituted with one to three substituents independently selected from hydroxy, halogen, C₁₋₆ alkyl, and C₁₋₆ alkoxy, wherein alkyl and alkoxy are unsubstituted or substituted with one to five halogens,
(CH₂)ₙ-heterocyclyl, wherein heterocyclyl is unsubstituted or substituted with one to three substituents independently selected from oxo, hydroxy, halogen, C₁₋₆ alkyl, and C₁₋₆ alkoxy, wherein alkyl and alkoxy are unsubstituted or substituted with one to five halogens, and
(CH₂)ₙ—C₃₋₆ cycloalkyl, wherein cycloalkyl is unsubstituted or substituted with one to three substituents independently selected from halogen, hydroxy, C₁₋₆ alkyl, and C₁₋₆ alkoxy, wherein alkyl and alkoxy are optionally substituted with one to five halogens;
wherein any methylene (CH₂) carbon atom in R⁸, R⁹, or R¹⁰ is unsubstituted or substituted with one to two groups independently selected from halogen, hydroxy, and C₁₋₄ alkyl unsubstituted or substituted with one to five halogens;
and R¹¹, R¹², and R¹³ are each independently hydrogen or methyl.

18. The compound of claim 17 wherein R⁸, R⁹, and R¹⁰ are each independently selected from the group consisting of
hydrogen,
C₁₋₃ alkyl, unsubstituted or substituted with one to three substituents independently selected from halogen, hydroxy, C₁₋₆ alkoxy, and phenyl-C₁₋₃ alkoxy, wherein alkoxy is unsubstituted or substituted with one to five halogens,
(CH₂)ₙ-phenyl, wherein phenyl is unsubstituted or substituted with one to five substituents independently selected from halogen, hydroxy, C₁₋₆ alkyl, and C₁₋₆ alkoxy, wherein alkyl and alkoxy are unsubstituted or substituted with one to five halogens,
(CH₂)ₙ-heteroaryl, wherein heteroaryl is unsubstituted or substituted with one to three substituents independently selected from hydroxy, halogen, C₁₋₆ alkyl, and C₁₋₆ alkoxy, wherein alkyl and alkoxy are optionally substituted with one to five halogens,
(CH₂)ₙ-heterocyclyl, wherein heterocyclyl is unsubstituted or substituted with one to three substituents independently selected from oxo, hydroxy, halogen, C₁₋₆ alkyl, and C₁₋₆ alkoxy, wherein alkyl and alkoxy are optionally substituted with one to five halogens, and
(CH₂)ₙ—C₃₋₆ cyclopropyl;
wherein any methylene (CH₂) carbon atom in R⁸, R⁹, or R¹⁰ is unsubstituted or substituted with one to two groups independently selected from halogen, hydroxy, and C₁₋₄ alkyl unsubstituted or substituted with one to five halogens;
and R¹¹, R¹², and R¹³ are each independently hydrogen or methyl.

19. The compound of claim 18 wherein R⁸, R⁹, and R¹⁰ are each independently selected from the group consisting of
hydrogen,
CH₃,
CH₂CH₃,
CH₂-cyclopropyl,
CHF-cyclopropyl,
CH(OH)-cyclopropyl,
CH₂OCH₂Ph,
CH₂(4-F-Ph),
CH₂(4-CF3-Ph), and
CH₂-[1,2,4]triazol-4-yl;
and R¹¹, R¹², and R¹³ are each independently hydrogen or methyl.

20. The compound of claim 18 wherein R⁹, R¹⁰, R¹², and R¹³ are hydrogen.

21. The compound of claim 20 wherein R⁸ and R¹¹ are hydrogen.

22. The compound of claim 21 which is selected from the group consisting of: 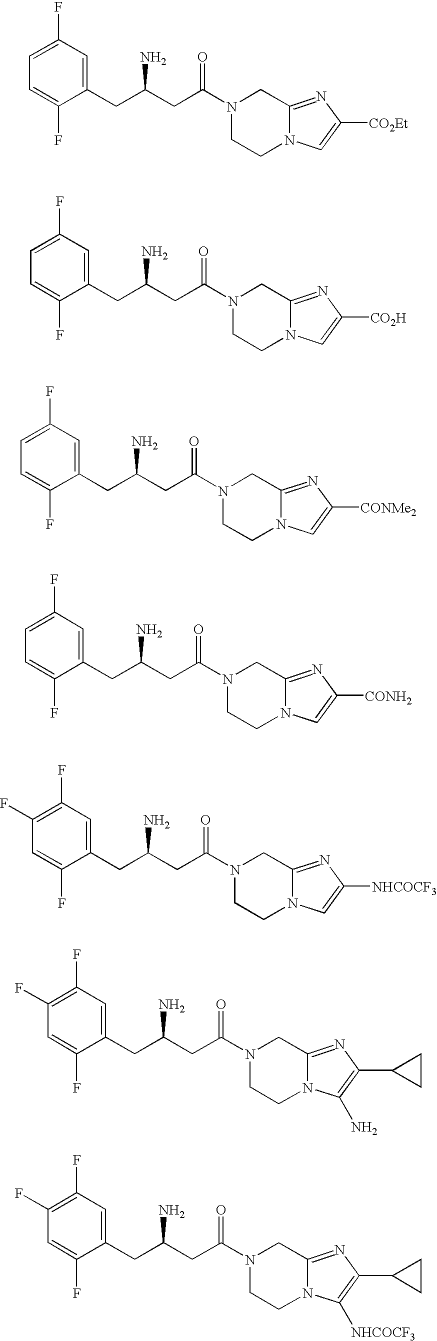 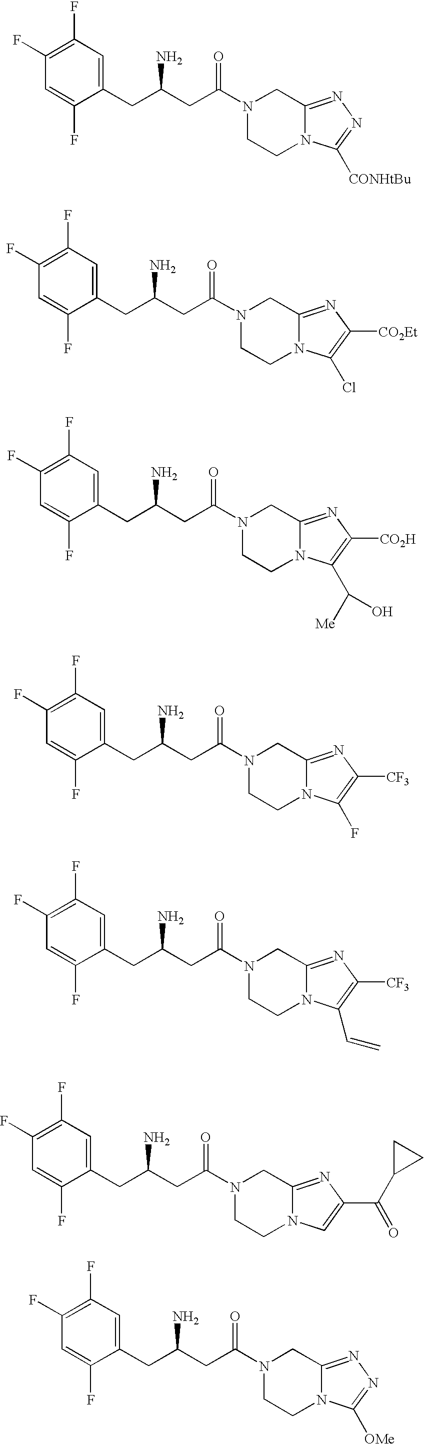 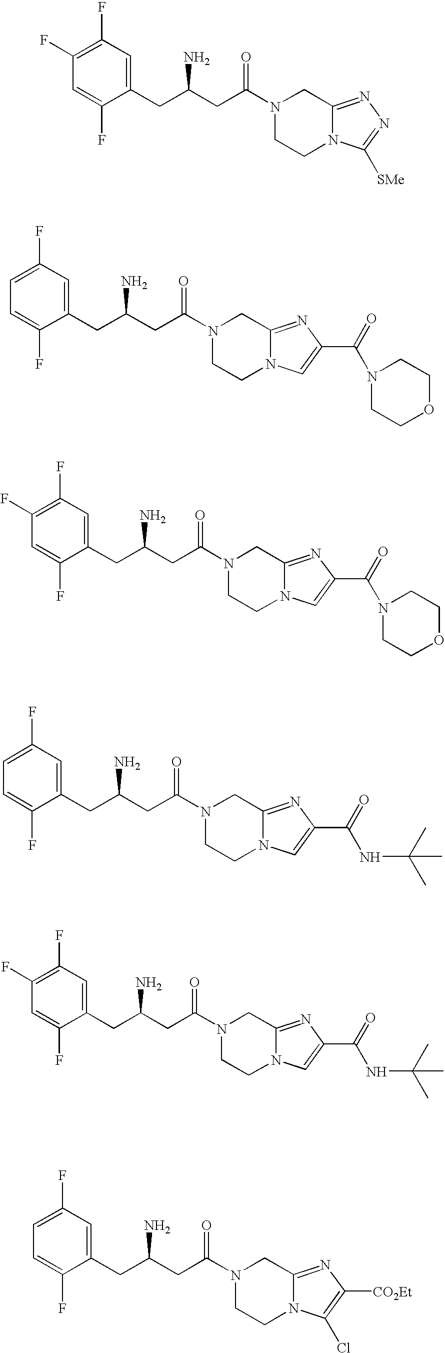 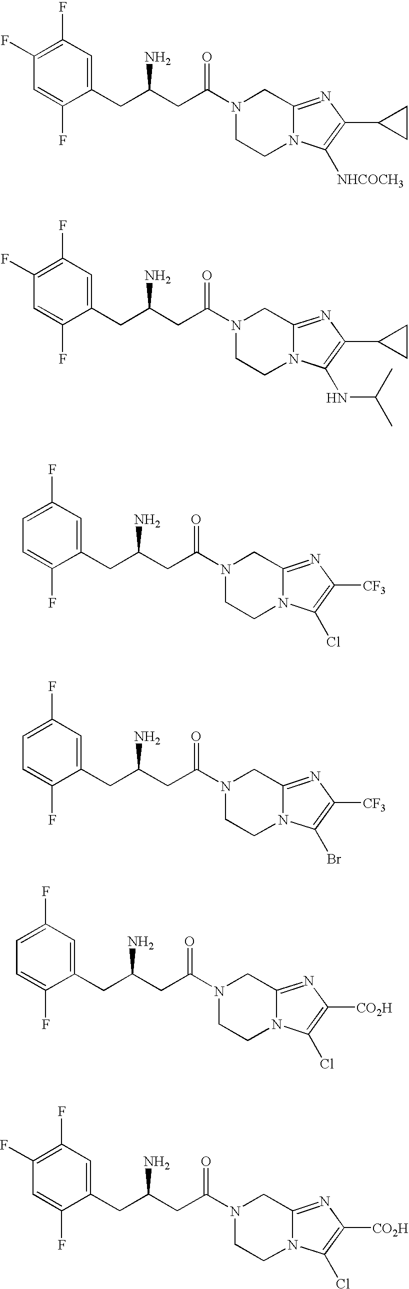 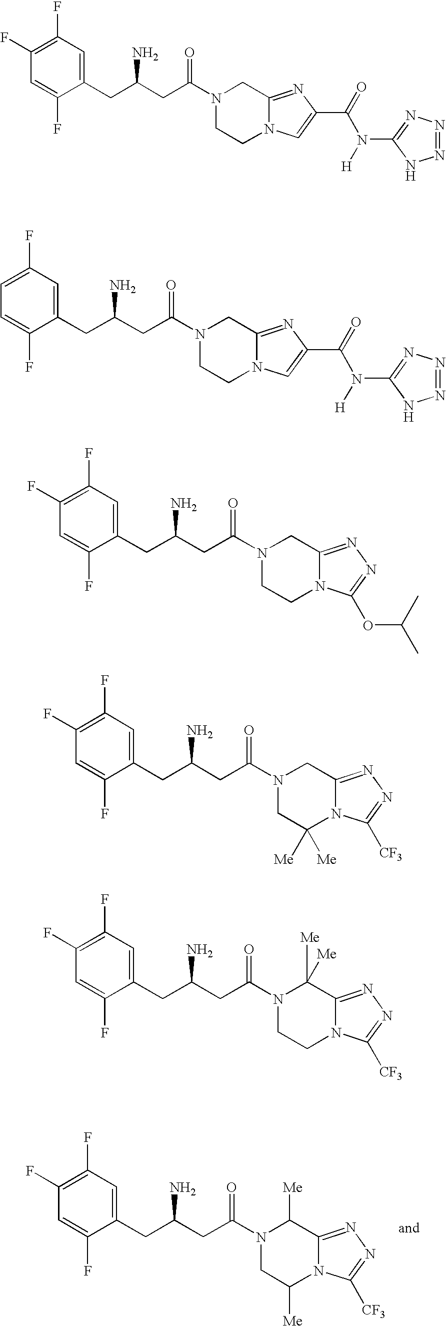 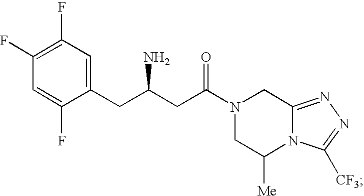
or a pharmaceutically acceptable salt thereof.

23. A pharmaceutical composition which comprises a compound of claim 1 and a pharmaceutically acceptable carrier.

24-25. (canceled)

26. A method for treating non-insulin dependent (Type 2) diabetes in a mammal in need thereof which comprises the administration to the mammal of a therapeutically effective amount of a compound of claim 1.

27. A method for treating hyperglycemia in a mammal in need thereof which comprises the administration to the mammal of a therapeutically effective amount of a compound of claim 1.

28. A method for treating obesity in a mammal in need thereof which comprises the administration to the mammal of a therapeutically effective amount of a compound of claim 1.

29. A method for treating one or more lipid disorders selected from the group of dyslipidemia, hyperlipidemia, hypertriglyceridemia, hypercholesterolemia, low HDL and high LDL in a mammal in need thereof which comprises the administration to the mammal of a therapeutically effective amount of a compound of claim 1.

30. (canceled)

31. The pharmaceutical composition of claim 23 further comprising one or more additional active ingredients selected from the group consisting of:
(a) a second dipeptidyl peptidase IV inhibitor;
(b) an insulin sensitizer selected from the group consisting of a PPARγ agonist, a PPARα/γ dual agonist, a PPARα agonist, a biguanide, and a protein tyrosine phosphatase-1B inhibitor;
(c) an insulin or insulin mimetic;
(d) a sulfonylurea or other insulin secretagogue;
(e) an α-glucosidase inhibitor;
(f) a glucagon receptor antagonist;
(g) GLP-1, a GLP-1 mimetic, or a GLP-1 receptor agonist;
(h) GIP, a GIP mimetic, or a GIP receptor agonist;
(i) PACAP, a PACAP mimetic, or a PACAP receptor agonist;
(j) a cholesterol lowering agent such as (i) HMG-CoA reductase inhibitor, (ii) sequestrant, (iii) nicotinyl alcohol, nicotinic acid or a salt thereof, (iv) PPARα agonist, (v) PPARα/γ dual agonist, (vi) inhibitor of cholesterol absorption, (vii) acyl CoA:cholesterol acyltransferase inhibitor, and (viii) anti-oxidant;
(k) a PPARδ agonist;
(l) an antiobesity compound;
(m) an ileal bile acid transporter inhibitor;
(n) an anti-inflammatory agent; and
(o) an antihypertensive agent.

32. (canceled)

33. A method of treating diabetes in a mammal in need thereof comprising administering to the mammal a therapeutically effective amount of a compound of claim 1 in combination with metformin.
